(19) United States
(12) Patent Application Publication
(10) Pub. No.: US 2010/0184989 A1
(43) Pub. Date: Jul. 22, 2010

(54) De Novo Synthesis of Conjugates

(75) Inventors: Jennifer Riggs-Sauthier, Huntsville, AL (US); Bo-Liang Deng, Madison, AL (US); Zhongxu Ren, Madison, AL (US); Wen Zhang, Madison, AL (US); Xuyuan Gu, Madison, AL (US); Franco J. Duarte, Huntsville, AL (US)

Correspondence Address: NEKTAR THERAPEUTICS, 201 INDUSTRIAL ROAD, SAN CARLOS, CA, 94070, US

(73) Assignee: Nektar Therapeutics, San Carlos, CA (US)

(21) Appl. No.: 12/530,438

(22) PCT Filed: Mar. 12, 2008
(86) PCT No.: PCT/US08/03351

§ 371 Date: Mar. 19, 2010

Related U.S. Application Data
(60) Provisional application No. 60/906,330, filed on Mar. 12, 2007.
(60) Provisional application No. 60/906,329, filed on Mar. 12, 2007.
(60) Provisional application No. 60/967,764, filed on Sep. 6, 2007.
(60) Provisional application No. 61/003,380, filed on Nov. 16, 2007.

(51) Int. Cl. A61K 47/48

(52) U.S. Cl. 546/282.1; 546/321; 548/318.1; 558/390; 560/26; 560/58

(57) ABSTRACT

The invention provides methods for the preparation of small molecule drugs that are chemically modified by covalent attachment of a water-soluble oligomer obtained from a water-soluble oligomer composition. Such drugs are produced through modification of a synthetic pathway to attach the oligomer to an intermediate compound followed by completion of the synthetic path.

### CROSS-REFERENCE TO RELATED APPLICATIONS

This application claims the benefit of the filing date, under 35 U.S.C. 119(e) of U.S. Provisional Application Ser. No. 60/906,330, filed Mar. 12, 2007; Ser. No. 60/967,764, filed Sep. 6, 2007; Ser. No. 60/906,329, filed Mar. 12, 2007; and Ser. No. 61/003,380, filed Nov. 16, 2007, each of which are hereby incorporated by reference in their entirety.

### FIELD OF THE INVENTION

The present invention relates to (among other things) novel synthetic methodologies for the preparation of poly- or oligo-ethylene glycol conjugates of pharmaceutically active compounds.

### BACKGROUND OF THE INVENTION

PEGylation can be defined as the act of covalently attaching a poly(ethylene glycol) (“PEG”) to a known active agent with the aim of forming a conjugate of the PEG and the active agent. Typically (and perhaps most conveniently), the known active agent is obtained (either commercially or synthetically) and a polymeric reagent is reacted with the active agent to form the conjugate.

Coupling of PEG derivatives is desirable to overcome obstacles encountered in the clinical use of biologically active molecules. Published PCT Publication No. WO 92/16221 states, for example, that many potentially therapeutic proteins have been found to have a short half life in the blood serum. PEGylation can decrease the rate of clearance from the bloodstream by increasing the apparent molecular weight of the molecule. Roughly, the rate of glomerular filtration of molecules is inversely related to the size of the molecule. The ability of PEGylation to decrease clearance, therefore, is generally not a function of how many PEG groups are attached to the molecule, but the overall molecular weight of the conjugate. Decreased clearance can lead to increased efficiency over the non-PEGylated material. See, for example, Conforti et al. (1987) Pharm. Research Commun. (19):287 and Katre et al. (1987) Proc. Natl. Acad. Sci. U.S.A. (84):1487.

Post-synthetic PEGylation of pharmaceutically active agents has been described in the art. Such processes, however, can add additional preparation time and costs to the production of PEG conjugates of pharmaceutically active compounds, particularly due to loss of yields from the requisite additional purification steps. Further, known pharmaceutically active agents may not have a synthetically accessible reactive group, making PEGylation of the molecule difficult, if not impossible.

In light of the preceding, there exists a need in the art for new methods for the production of PEG conjugates of pharmaceutically active compounds.

### SUMMARY OF THE INVENTION

Attempts to prepare conjugates in a single step without the use of protecting groups, while alluring, can result in attachment of multiple PEG molecules and/or random attachment of PEG molecules, often leading to loss of pharmacologic activity. In addition, if the PEGylated conjugate is intended for therapeutic use, the multiple species mixture that results from non-specific PEGylation leads to difficulties in the preparation of a product with reproducible and characterizable properties. This non-specific PEGylation makes it difficult to evaluate therapeutics and to establish efficacy and dosing information.

In one or more embodiments of the invention, a method of synthesizing a conjugate of a pharmaceutically active compound is provided, the method comprising: attaching at least one water-soluble oligomer, directly or through a linker group, at one or more synthetically available positions within an intermediate compound; and completing a synthetic path to yield the conjugate of the pharmaceutically active compound.

In one or more embodiments of the invention, a method for synthesizing a conjugate of a pharmaceutically active compound comprising is provided, the method comprising: selecting a pharmaceutically active compound having a synthetic path; modifying the synthetic path by attaching at least one oligoethylene glycol residue, directly or through a linker group, at one or more synthetically available positions within one or more intermediate compounds of the synthetic path; and completing the synthetic path to yield the conjugate of the pharmaceutically active compound.

The methods of the invention advantageously provide (among other things) a “de novo” synthesis wherein an intermediate is covalently attached with a water-soluble oligomer and the remainder of the conjugate is subsequently synthesized from the water-soluble oligomer-intermediate. In this way, it can be possible to selectively attach a water-soluble polymer at a location within a pharmaceutically active agent that might not otherwise be available for covalent attachment with a water-soluble oligomer and avoid costly post-synthesis chemical modification of the active agent. In particular, methods herein enable site selective attachment of a water-soluble oligomer to pharmaceutically active compounds that could lead to reproducibly-modified materials that gain the desirable attributes of conjugation without the loss of activity. Thus, one of the improvements the present invention provides over prior art methods of conjugation is attaching the water-soluble oligomer to a precursor of the pharmaceutically active agent.

### DETAILED DESCRIPTION OF THE INVENTION

Unless otherwise indicated, an “oligoethylene glycol residue” (also called a “PEG oligomer”) is one in which substantially all (and more preferably all) monomeric subunits are ethylene oxide subunits. The oligoethylene glycol residue can contain distinct end groups such as methyl or unfunctionalized groups and functional groups, such as a carboxylic acid, activated carboxylic acid, amines, hydroxyl, or thiol. Typically, PEG oligomers for use in the present invention will comprise one of the two following structures: “—(CH₂CH₂O)ₙ—” or “—(CH₂CH₂O)ₙ₋₁CH₂CH₂—,” depending upon whether the terminal oxygen(s) has been displaced, e.g., during a synthetic transformation. For PEG oligomers, “n” varies from about 2 to 50, preferably from about 2 to about 30, more preferably from about 2 to about 12, and even more preferably from about 2 to 8, and, in particular 2, 3, 4, 5, 6, 7, or 8. When PEG further comprises a functional group, A, for linking to, e.g., a small molecule drug, the functional group when covalently attached to a PEG oligomer does not result in formation of (i) an oxygen-oxygen bond (—O—O—, a peroxide linkage), or (ii) a nitrogen-oxygen bond (N—O, O—N).

As used herein, an “intermediate compound” is any compound in a synthetic path which is not the final synthetic product. For example, intermediate compounds include starting materials.

As used herein, “functional groups” are any chemical moiety other than a hydrocarbon moiety (i.e., “unfunctionalized groups”), including, but not limited to, carboxylic acids, activated carboxylic acids, amides, esters, ethers, thioethers, amines, imines, hydroxyls, thiols, electrophilic unsaturated bonds (e.g., malimides, and other Michael acceptors) and chemically accessible carbon atoms (e.g., primary) having at least one “nucleophilic leaving group” as defined herein.

As used herein a “synthetically available position” is any position within a molecule which can be chemically modified to introduce an oligoethylene glycol residue as described herein. Synthetically available positions include, but are not limited to, unfunctionalized positions (i.e., a position occupied by a hydrogen atom), carboxylic acids, activated carboxylic acids, amides, esters, ethers, thioethers, amines, imines, hydroxyls, thiols, electrophilic unsaturated bonds (e.g., malimides, and other Michael acceptors) and chemically accessible carbon atoms (e.g., primary) having at least one “nucleophilic leaving group” as defined herein. For example, an ether group, such as a methoxy group, can be replaced or modified to introduce an oligoethylene glycol group. In certain embodiments, a synthetically available position can have a hydrogen atom whose pKa is about 25 or less. In certain other embodiments, a synthetically available position is an unfunctionalized position.

“Nucleophilic leaving groups” as used herein are those known to those skilled in the art that can be displaced by a nucleophile in a nucleophilic substitution reaction. Such groups include, but are not limited to, chloro, bromo, iodo, tosyl, brosyl, mesyl, noflyl, and triflyl.

In the methods of the invention, it is generally preferred that at least one oligoethylene glycol residue is attached to at least one intermediate compound of the known synthetic path of the known pharmaceutically active compound.

Synthetic paths for synthesis of a water-soluble oligomer-conjugated active agent (e.g., a PEGylated active agent) can be, for example, a convergent path having two intermediate compounds that are reacted to yield the pharmaceutically active compound or a protected form of the pharmaceutically active compound, wherein the synthetically available position is within at least one of the two intermediate compounds. In some embodiments, oligoethylene glycol residues are attached at a synthetically available position within both intermediate compounds.

In addition, the synthetic path can be a linear path; in such cases, at least one intermediate compound is attached to at least one water-soluble oligomer (e.g., an oligoethylene glycol residue). In some embodiments, at least two intermediates are each attached to at least one water-soluble oligomer (e.g., oligoethylene glycol residue), such that the conjugate obtained upon completion of the synthetic path comprises at least two oligoethylene glycol residues.

Each water-soluble oligomer may be composed of up to three different monomer types selected from the group consisting of: alkylene oxide, such as ethylene oxide or propylene oxide; olefinic alcohol, such as vinyl alcohol, 1-propenol or 2-propenol; vinyl pyrrolidone; hydroxyalkyl methacrylamide or hydroxyalkyl methacrylate, where alkyl is preferably methyl; α-hydroxy acid, such as lactic acid or glycolic acid; phosphazene, oxazoline, amino acids, carbohydrates such as monosaccharides, alditol such as mannitol; and N-acryloylmorpholine. Preferred monomer types include alkylene oxide, olefinic alcohol, hydroxyalkyl methacrylamide or methacrylate, N-acryloylmorpholine, and α-hydroxy acid. Preferably, each oligomer is, independently, a co-oligomer of two monomer types selected from this group, or, more preferably, is a homo-oligomer of one monomer type selected from this group.

The two monomer types in a co-oligomer may be of the same monomer type, for example, two alkylene oxides, such as ethylene oxide and propylene oxide. Preferably, the oligomer is a homo-oligomer of ethylene oxide. Usually, although not necessarily, the terminus (or termini) of the oligomer that is not covalently attached to a small molecule is capped to render it unreactive. Alternatively, the terminus may include a reactive group. When the terminus is a reactive group, the reactive group is either selected such that it is unreactive under the conditions of formation of the final oligomer or during covalent attachment of the oligomer to a small molecule drug, or it is protected as necessary. One common end-functional group is hydroxyl or —OH, particularly for oligoethylene oxides.

Generally, an oligoethylene glycol residue can be attached to an intermediate at any synthetically available position in the one or more intermediate compounds, e.g., a synthetically available position that is one with a hydrogen having a pKa of less than about 25. Preferably, the oligoethylene glycol residue is attached to the intermediate through an ether, thioether, ester, thioester, amide, carbonate, carbamate, urea, imino, or amino bond.

In some embodiments, each of the one or more oligoethylene glycol residues is conjugated to an active agent by contacting one or more intermediate compounds at one or more synthetically available positions with one or more oligoethylene glycol residue source compounds each independently of the formula

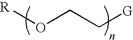

wherein n is an integer having a value of from 2 to 50 (e.g., 2, 3, 4, 5, 6, 7, or 8); R is selected from the group consisting of —OH, C₁-C₁₀ alkyl, and hydroxy-protecting groups; and G is selected from the group consisting of nucleophilic leaving groups, —OH, —SH, —NH₂, —NH(C₁-C₆ alkyl), —C(O)OH, —C(O)OC₁-C₆alkyl, and activated carboxylic acid groups.

A linker group, having at least two synthetically available linker positions, can be attached at any synthetically available position within any of the intermediate compounds to facilitate introduction of the water-soluble oligomer (e.g, oligoethylene glycol residue), either for providing an appropriate functional group to the intermediate compound and/or for providing physical separation between, ultimately, the pharmaceutically active compound, and any or all of the water-soluble oligomers (e.g., oligoethylene glycol residues). After attachment of the linker group to the intermediate, a water-soluble oligomer (e.g., an oligoethylene glycol residue) can be attached to the linker via a synthetically available linker position to provide the intermediate modified with a water-soluble oligomer (e.g., an oligoethylene glycol residue).

In certain embodiments, the linker group can comprise two synthetically available linker positions, wherein one of the synthetically available linker positions is optionally protected. In some embodiments, the methods provide for attaching such a linker group to a synthetically available position of the intermediate compound via an unprotected synthetically available linker position, deprotecting the protected synthetically available linker position, and attaching a water-soluble oligomer (e.g., an oligoethylene glycol residue) to the deprotected synthetically available linker position. Deprotecting the protected synthetically available linker position, and attaching a water-soluble oligomer (e.g., an oligoethylene glycol residue) to the deprotected synthetically available linker position can be conducted before or after synthesis of the active agent part of the conjugate is completed.

Alternatively, the methods provide for attaching one or more water-soluble oligomers (e.g., oligoethylene glycol residues) to a linker group having two or more synthetically available linker positions, and attaching the linker group with the attached water-soluble oligomer (e.g., oligoethylene glycol residue) to an intermediate compound at one or more synthetically available positions.

In some instances, the linker group “L” comprises an ether, amide, urethane, amine, thioether, urea, or a carbon-carbon bond. Functional groups such as those discussed below, and illustrated in the examples, are typically used for forming the linkages. The linker moiety may less preferably also comprise (or be adjacent to or flanked by) other atoms, as described further below.

More specifically, in selected embodiments, a linker moiety of the invention, L, may be any of the following: “-” (i.e., a covalent bond, that may be stable or degradable), —O—, —NH—, —S—, —C(O)—, C(O)—NH, NH—C(O)—NH, O—C(O)—NH, —C(S)—, —CH₂—, —CH₂—CH₂—, —CH₂—CH₂—CH₂—, —CH₂—CH₂—CH₂—CH₂—, —O—CH₂—, —CH₂—O—, —O—CH₂—CH₂—, —CH₂—O—CH₂—, —CH₂—CH₂—O—, —O—CH₂—CH₂—CH₂—, —CH₂—O—CH₂—CH₂—, —CH₂—CH₂—O—CH₂—, —CH₂—CH₂—CH₂—O—, —O—CH₂—CH₂—CH₂—CH₂—, —CH₂—O—CH₂—CH₂—CH₂—, —CH₂—CH₂—O—CH₂—CH₂—, —CH₂—CH₂—CH₂—O—CH₂—, —CH₂—CH₂—CH₂—CH₂—O—, —C(O)—NH—CH₂—, —C(O)—NH—CH₂—CH₂—, —CH₂—C(O)—NH—CH₂—, —CH₂—CH₂—C(O)—NH—, —C(O)—NH—CH₂—CH₂—CH₂—, —CH₂—C(O)—NH—CH₂—CH₂—, —CH₂—CH₂—C(O)—NH—CH₂—, —CH₂—CH₂—CH₂—C(O)—NH—, —C(O)—NH—CH₂—CH₂—CH₂—CH₂—, —CH₂—C(O)—NH—CH₂—CH₂—CH₂—, —CH₂—CH₂—C(O)—NH—CH₂—CH₂—, —CH₂—CH₂—CH₂—C(O)—NH—CH₂—, —CH₂—CH₂—CH₂—C(O)—NH—CH₂—CH₂—, —CH₂—CH₂—CH₂—CH₂—C(O)—NH—, —NH—C(O)—CH₂—, —CH₂—NH—C(O)—CH₂—, —CH₂—CH₂—NH—C(O)—CH₂—, —NH—C(O)—CH₂—CH₂—, —CH₂—NH—C(O)—CH₂—CH₂, —CH₂—CH₂—NH—C(O)—CH₂—CH₂, —C(O)—NH—CH₂—, —C(O)—NH—CH₂—CH₂—, —O—C(O)—NH—CH₂—, —O—C(O)—NH—CH₂—CH₂—, —NH—CH₂—, —NH—CH₂—CH₂—, —CH₂—NH—CH₂—, —CH₂—CH₂—NH—CH₂—, —C(O)—CH₂—, —C(O)—CH₂—CH₂—, —CH₂—C(O)—CH₂—, —CH₂—CH₂—C(O)—CH₂—, —CH₂—CH₂—C(O)—CH₂—CH₂—, —CH₂—CH₂—C(O)—, —CH₂—CH₂—CH₂—C(O)—NH—CH₂—CH₂—NH—, —CH₂—CH₂—CH₂—C(O)—NH—CH₂—CH₂—NH—C(O)—, —CH₂—CH₂—CH₂—C(O)—NH—CH₂—CH₂—NH—C(O)—CH₂—, bivalent cycloalkyl group, —N(R⁶)—, R⁶ is H or an organic radical selected from the group consisting of alkyl, substituted alkyl, alkenyl, substituted alkenyl, alkynyl, substituted alkynyl, aryl and substituted aryl.

Additional linker moieties include, acylamino, acyl, aryloxy, alkylene bridge containing between 1 and 5 inclusive carbon atoms, alkylamino, dialkylamino having about 2 to 4 inclusive carbon atoms, piperidino, pyrrolidino, N-(lower alkyl)-2-piperidyl, morpholino, 1-piperizinyl, 4-(lower alkyl)-1-piperizinyl, 4-(hydroxyl-lower alkyl)-1-piperizinyl, 4-(methoxy-lower alkyl)-1-piperizinyl, and guanidine. In some instances, it is preferred that L is not an amide, i.e., —C(O)N(R)— or —(R)NC(O)—.

For purposes of the present invention, however, a group of atoms is not considered a linker when it is immediately adjacent to an polymer segment, and the group of atoms is the same as a monomer of the polymer such that the group would represent a mere extension of the polymer chain.

In some embodiments, the one or more oligoethylene glycol residues are introduced by contacting one or more intermediate compounds at one or more synthetically available positions with one or more oligoethylene glycol residue source compounds each independently of the formula,

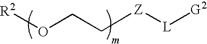

wherein m is 2, 3, 4, 5, 6, 7, or 8; Z is —O— or —N(H)—; R² is C₁-C₁₀ alkyl or a hydroxy-protecting group; L is —C(O)—, —C₁-C₆ alkyl-, —C(O)C₁-C₆ alkyl-, —C(O)OC₁-C₆ alkyl-, or —C(O)N(H)C₁-C₆ alkyl-; and G² is halogen, —OH, —SH, —NH₂, —NH(C₁-C₆ alkyl), —C(O)OH, —C(O)OC₁-C₆alkyl, or an activated carboxylic acid group.

A “protecting group” is a moiety that prevents or blocks reaction of a particular chemically reactive functional group in a molecule under certain reaction conditions. The protecting group will vary depending upon the type of chemically reactive group being protected as well as the reaction conditions to be employed and the presence of additional reactive or protecting groups in the molecule. Functional groups which may be protected include, by way of example, carboxylic acid groups, amino groups, hydroxyl groups, thiol groups, carbonyl groups and the like. Representative protecting groups for carboxylic acids include esters (such as a p-methoxybenzyl ester), amides and hydrazides; for amino groups, carbamates (such as tert-butoxycarbonyl) and amides; for hydroxyl groups, ethers and esters; for thiol groups, thioethers and thioesters; for carbonyl groups, acetals and ketals; and the like. Such protecting groups are well-known to those skilled in the art and are described, for example, in T. W. Greene and G. M. Wuts, Protecting Groups in Organic Synthesis, Third Edition, Wiley, New York, 1999, and references cited therein. In particular, “hydroxy-protecting groups” include, but are not limited to, benzyl (Bn), substituted benzyl, methoxymethyl (MOM), trimethylsilyl (TMS), triisopropylsilyl (TIPS), t-butyldimethylsilyl (TBDMS), benzyloxymethyl (BOM), substituted benzyloxymethyls (e.g., p-nitrobenzyloxymethyl), t-butyoxymethyl, tetrahydropyranyl (THP), t-butyl, allyl, acetyl, trifluoroacetyl, benzoyl, methoxycarbonyl, t-butoxycarbonyl (BOC), 9-fluorenylmethylcarbonyl (Fmoc), and 2,2,2-trichloroethyloxycarbonyl (Troc).

The following table (Table A) shows various methods for introducing an oligoethylene glycol residue into an intermediate according to the present invention (corresponding approaches for a water-soluble oligomers can also be used). R, R², Z, L, G, G², m, and n can have the definitions as provided above; R′ can be hydrogen or any functional group which will not interfere with the reaction, for example, lower alkyl, benzyl, and the like. “Non-interfering substituents” are those groups that, when present in a molecule, are typically non-reactive with other functional groups contained within the molecule. R* is an activated carboxylic acid derivative, and X is a halogen.

TABLE A

| 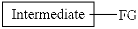 FG | 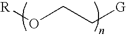 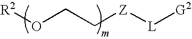 G/G² | Sample Reaction conditions | Product |
|---|---|---|---|
| —OH —Cl, —Br, —I, —OTs, —OMs, —ONf, | —OH —Cl, —Br, —I, —OTs, —OMs, —ONf, —OH | Mitsunobu Williamson ether synthesis | 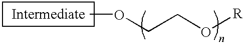 |
| —C(O)OH —C(O)X —C(O)R* —C(O)OR′ | —OH | Esterification Transesterification | 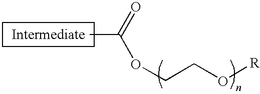 |
| —C(O)OH —C(O)X —C(O)R* | —NH(R′) | Amidation | 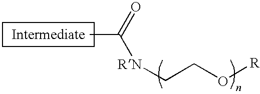 |
| —NH(R′) | —C(O)OH —C(O)X —C(O)R* | Amidation | 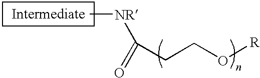 |
| —OH | —C(O)OH —C(O)X —C(O)OR′ | Esterification Transesterification | 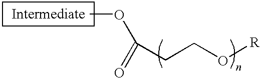 |
| —NH₂ —C(O)H | —C(O)H —NH₂ | Imine formation | 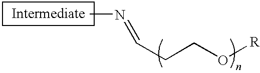 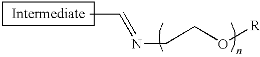 |
| —N═C═O | —NHR′ | Urea, Thiourea formation | 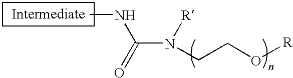 |
| —N═C═S | —NHR′ | | 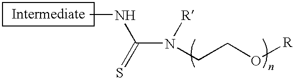 |
| —NHR′ | —N═C═O | | 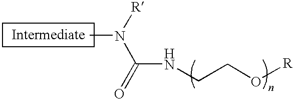 |
| | —N═C═S | | 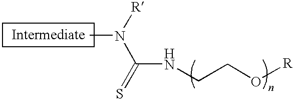 |
| —OH | —N═C═O | Carbamate formation | 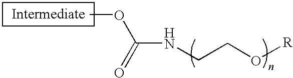 |
| —N═C═O | —OH | | 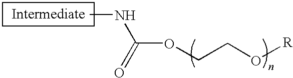 |
| —SH | 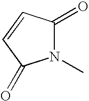 | Michael addition | 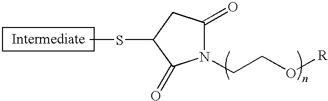 |
| —SH —Cl, —Br, —I, —OTs, —OMs, —ONf, | —Cl, —Br, —I, —Ots, —OMs, —ONf, —SH | Thiol alkylation | 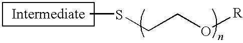 |

General reaction conditions for the preceding reactions can be found, for example, in LaRock, Comprehensive Organic Transformations, 2^{nd} ed., Wiley-VCH: New York, 1999; and March, Advanced Organic Chemistry, 4^{th} ed., Wiley: New York, 1992, each of which are hereby incorporated by reference in their entirety.

An “activated carboxylic acid derivative” refers to a carboxylic acid derivative that reacts readily with nucleophiles, generally much more readily than the underivatized carboxylic acid. Activated carboxylic acids include, for example, acid halides (such as acid chlorides), anhydrides, carbonates, and esters. Such esters include imide esters, of the general form —(CO)O—N[(CO)—]₂; for example, N-hydroxysuccinimidyl (NHS) esters or N-hydroxyphthalimidyl esters. Also preferred are imidazolyl esters and benzotriazole esters. Particularly preferred are activated propionic acid or butanoic acid esters, as described in co-owned U.S. Pat. No. 5,672,662. These include groups of the form —(CH₂)₂₋₃C(═O)O-Q, where Q is preferably selected from N-succinimide, N-sulfosuccinimide, N-phthalimide, N-glutarimide, N-tetrahydrophthalimide, N-norbornene-2,3-dicarboximide, benzotriazole, 7-azabenzotriazole, and imidazole. Other activated carboxylic acid groups include succinimidyl carbonate, maleimide, benzotriazole carbonate, glycidyl ether, imidazoyl carbonate, p-nitrophenyl carbonate, acrylate, tresylate, aldehyde, and orthopyridyl disulfide.

As necessary, the preceding intermediates containing at least one water-soluble oligomer (e.g., oligoethylene glycol residue) can be further reacted, prior to completion of the synthetic path, to modify the bonding between the water-soluble oligomer (e.g., oligoethylene glycol residue) and the intermediate. For example, imino groups can be chemically reduced to increase the hydrolytic stability of the conjugate (e.g., Table B).

TABLE B

| Intermediate | Conditions | Product |
|---|---|---|
| 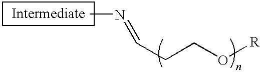 | Imine reduction (e.g., NaBH₃CN) | 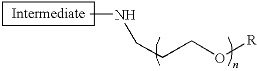 |
| 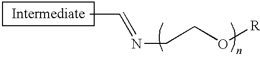 | | 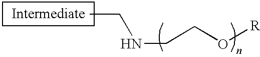 |

In such embodiments, the invention provides the advantage of allowing the selective introduction of water-soluble oligomers (e.g., oligoethylene glycol residues) without adding steps to the end of the synthetic pathway. Such steps can generally decrease synthetic yields due to losses in side reactions and/or purification. For example, when the desired pharmaceutically active compound contains multiple functional groups that can be modified with one or more water-soluble oligomers (e.g., oligoethylene glycol residues), the methods of the invention provide the ability to selectively modify the pharmaceutically active compound at one or more synthetically available position without the need to add protecting and deprotecting steps.

Preferably, the conjugates of the invention comprise the at least one oligoethylene glycol residue at one or more positions that are not available in the pharmaceutically active compound. “Not available” as used herein means that the position is either physically unavailable due to, for example, (i) steric considerations of the overall structure and/or conformation of the pharmaceutically active compound; (ii) the presence of multiple reactive groups in the pharmaceutically active compound that prevent selective modification of the compound; (iii) the group otherwise having been modified as a result of the synthetic path and is not generally reactive without additional chemical steps. A position “not available” in the pharmaceutically active compound is one to which a water-soluble oligomer (e.g., an oligoethylene glycol residue) cannot readily be attached when reacting the pharmaceutically active compound per se (or an activated/reactive counterpart thereof) with a water-soluble oligomer (e.g., an oligoethylene glycol residue) (or an activated/reactive counterpart thereof). An advantage of the methods of the present invention is that one can make water-soluble oligomer-active agent conjugates having structures not synthesizable (or not readily synthesizable) by prior art methods of reacting water-soluble oligomers with the fully formed active agent.

For example, in case (ii), a conjugate can comprise the at least one residue at one or more positions which are not available in the pharmaceutically active compound without protecting one or more functional groups in the pharmaceutically active compound.

In another example, in case (iii), an alcohol group from one intermediate has been transformed into a methyl ether group as a result of the known synthetic path, modification of the site would require removal of the methyl group and subsequent reaction of the alcohol to introduce the oligoethylene glycol residue. In the present methods, the alcohol in the intermediate can be reacted to introduce the oligoethylene glycol residue which may function in an equivalent sense as the methyl ether for the purposes of the remainder of the known synthetic path (e.g., acting as a protecting group).

In certain preferred embodiments, the pharmaceutically active compound is a protease inhibitor, opioid receptor agonist, anticholineric, muscle relaxant, calcium channel blocker, or an anti-viral. For example, the pharmaceutically active compound is selected from the group consisting of nifedipine, verapamil, dantrolene, oxybutynin, BW373U86, atazanavir, darunavir, tipranavir, and foscarnet. In another embodiment, the pharmaceutically active compound is an active agent described in U.S. Patent Application Publication No. 2005/0136031.

In any of the preceding aspects and embodiments of the invention, preferably, at least one of the intermediate compounds to which one or more water-soluble oligomers is attached does not have any known pharmacological activity. In any of the preceding aspects and embodiments of the invention, preferably, at least one of the intermediate compounds to which one or more water-soluble oligomers is attached does not have any substantial pharmacological activity. In one or more embodiments of the invention, at least one of the intermediate compounds to which one or more water-soluble oligomers is attached is toxic (e.g., at the same molar dose as the active agent). In one or more embodiments of the invention, at least one of the intermediate compounds to which one or more water-soluble oligomers is not indicated for the use or uses indicated by the active agent.

As used in this specification, the singular forms “a,” “an,” and “the” include plural referents unless the context clearly dictates otherwise.

“Alkyl” refers to a hydrocarbon chain, typically ranging from about 1 to 20 atoms in length. Such hydrocarbon chains are preferably but not necessarily saturated and may be branched or straight chain, although typically straight chain is preferred. Exemplary alkyl groups include methyl, ethyl, propyl, butyl, pentyl, 1-methylbutyl, 1-ethylpropyl, 3-methylpentyl, and the like. As used herein, “alkyl” includes cycloalkyl when three or more carbon atoms are referenced. An “alkenyl” group is an alkyl of 2 to 20 carbon atoms with at least one carbon-carbon double bond.

“Lower alkyl” refers to an alkyl group containing from 1 to 6 carbon atoms, and may be straight chain or branched, as exemplified by methyl, ethyl, n-butyl, i-butyl, and t-butyl. “Lower alkenyl” refers to a lower alkyl group of 2 to 6 carbon atoms having at least one carbon-carbon double bond.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g., CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding multivalent moiety, arylene). All atoms are understood to have their normal number of valences for bond formation (i.e., 1 for H, 4 for carbon, 3 for N, 2 for O, and 2, 4, or 6 for S, depending on the oxidation state of the S).

### EXPERIMENTAL

It is to be understood that while the invention has been described in conjunction with certain preferred and specific embodiments, the foregoing description as well as the examples that follow are intended to illustrate and not limit the scope of the invention. Other aspects, advantages and modifications within the scope of the invention will be apparent to those skilled in the art to which the invention pertains.

All chemical reagents referred to in the appended examples are commercially available unless otherwise indicated. The preparation of PEG-mers is described in, for example, U.S. Patent Application Publication No. 2005/0136031. All oligo(ethylene glycol) methyl ethers employed in the Examples below were monodisperse and chromatographically pure, as determined by reverse phase chromatography.

### Example 1

### De Novo Synthesis of PEG-Nifedipine

### Approach A

PEG-Nifedipine was prepared using a first approach. Schematically, the approach followed for this example is shown below (compound numbers in bold in the schematic correspond to the compound numbers provided in the text of this Example 1 alone).

De Novo Synthesis of PEG-Nifedipine—“Approach A” Schematic

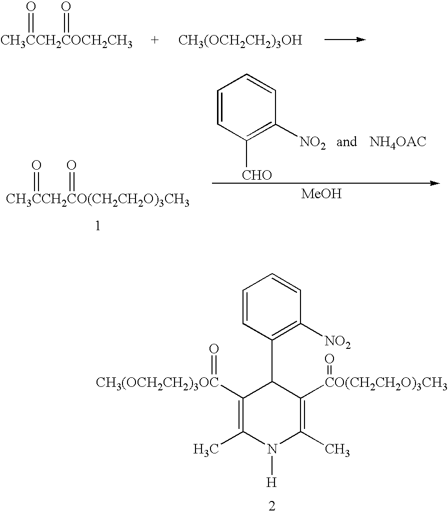

### Synthesis of methyl tri(ethylene glycol)acetoacetate (1)

Tri(ethylene glycol)monomethyl ether (8.2 g, 50 mmol) and ethyl acetoacetate (9.75 g, 75 mmol) were heated to 180° C. for three hours and then ethanol and the excess ethyl acetoacetate were distilled out at 160° C. by reduced pressure distillation. The product (1) (11.16 g, yield 90%) is pure by NMR and used directly for the next step. ¹H NMR (CDCl3): δ 4.30 (t, 2H), 3.73-3.53 (m, 10H), 3.48 (s, 2H), 3.38 (s, 3H), 2.27 (s, 3H).

### Synthesis of 2,6-dimethyl 4-(2-nitrophenyl)-1,4-dihydropyridine-3,5-dicarboxylic acid dimethoxy tri(ethylene glycol)ester (2)

Methyl tri(ethylene glycol)acetoacetate (1) (496 mg, 2.0 mmol), 2-nitrobenzylaldehyde (151 mg, 1.0 mmol), and ammonium acetate (77 mg, 1.0 mmol) were dissolved in methanol (10 ml). The reaction was heated to reflux for two days. The solvent was evaporated and the residue was subjected to flash chromatography (acetone/ethyl acetate=2%˜4%) to obtain compound (2) (45 mg, yield 8%). ¹H NMR (CDCl3): δ 7.72 (d, 1H), 7.51-7.45 (m, 2H), 7.26 (d, 1H), 5.90 (s, 1H), 5.84 (s, 1H), 4.27-4.21 (m, 2H), 4.10-4.05 (m, 2H), 3.66-3.52 (m, 20H), 3.38 (s, 6H), 2.32 (s, 6H). LC/MS: 628 [M+NH₄]⁺, 633 [M+Na]⁺.

### Example 2

### Calcium Channel Binding Assay

A calcium channel type L binding assay was performed having the following characteristics: K_{D} (binding affinity)=0.20 nM; Bₘₐₓ (receptor number): 166 fmol/mg tissue (wet weight). In the assay, rat cortical membranes were used as a receptor source and the radioligand [³H]Nitrendipine (70-87 Ci/mmol) was used at a final ligand concentration of 0.2 nM. The non-specific determinant was nifedipine (0.1 μM) and both the reference compound and positive control was nifedipine. The reactions were carried out in 50 mM TRIS-HCl (pH 7.7) at 25° C. for 60 minutes. The reaction was terminated by rapid vacuum filtration onto glass fiber filters. Radioactivity trapped onto the filters was determined and compared to control values in order to ascertain any interactions of test compound with the nitredipine binding site (Gould, Murphy, and Snyder. Molecular Pharmacology 25, 235-241 (1984)). The results are shown below. In the assay: nifedipine exhibited an IC₅₀ 1.7×10⁻⁹ and compound (2) from Example 1 had an IC₅₀ of 1.6×10⁻⁷; nifedipine exhibited an IC₅₀ 1.88×10⁻⁹ and compound (6a) from Example 3 had an IC₅₀ of 6.74×10⁻⁸; nifedipine exhibited an IC₅₀ 2.12×10⁻⁹ and compound (6b) from Example 3 had an IC₅₀ of 1.55×10⁻⁸; nifedipine exhibited an IC₅₀ 1.77×10⁻⁹ and compound (6c) from Example 3 had an IC₅₀ of 5.56×10⁻⁸.

### Example 3

### De novo Synthesis of PEG-Nifedipine

### Approach B

PEG-Nifedipine was prepared using a second approach. Schematically, the approach followed for this example is shown below (compound numbers in bold in the schematic correspond to the compound numbers provided in the text of this Example 3 alone).

De Novo Synthesis of PEG-Nifedipine—“Approach B” Schematic

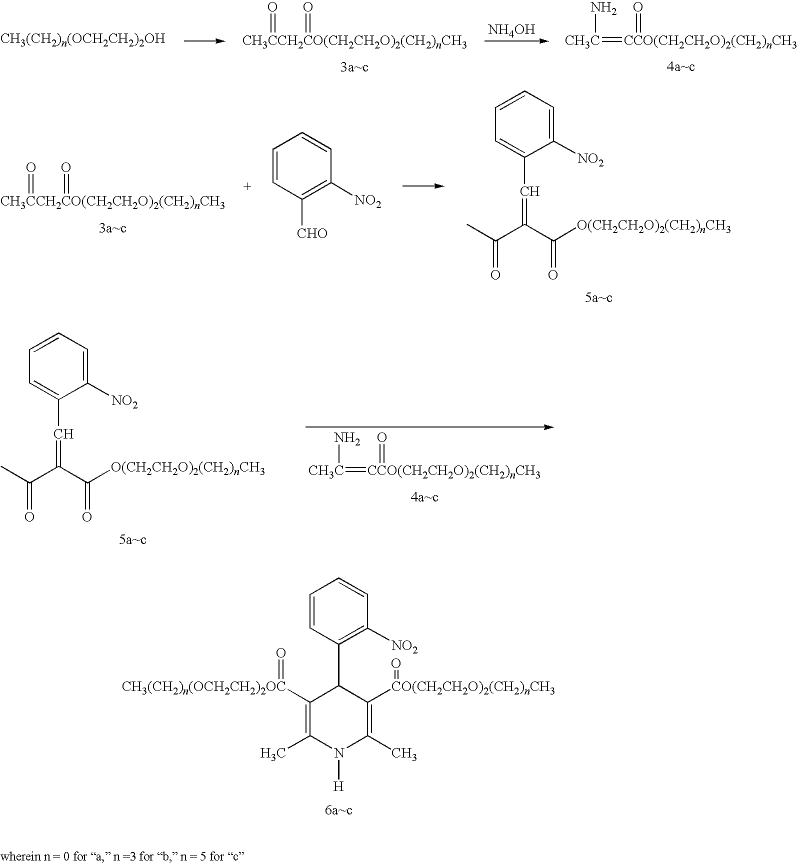

### Synthesis of methyl di(ethylene glycol)acetoacetate (3a)

Di(ethylene glycol)monomethyl ether (6.0 g, 50 mmol) and ethyl acetoacetate (9.75 g, 75 mmol) were heated to 180° C. for 3 hours and then ethanol and the excess ethyl acetoacetate were distilled out at 160° C. by reduced pressure distillation. The product (3a) (9.2 g, yield 90%) is pure by NMR and used directly for the next step. ¹H NMR (CDCl3): δ 4.30 (t, 2H), 3.69 (t, 2H), 3.60 (t, 2H), 3.52 (t, 2H), 3.46 (s, 2H), 3.35 (s, 3H), 2.25 (s, 3H).

### Synthesis of methyl di(ethylene glycol) 3-aminocrotonate (4a)

Methyl di(ethylene glycol)acetoacetate (3a) (1.02 g, 5.0 mmol), ammonium hydroxide (0.78 ml, 6.0 mmol), and silica gel powder (60 mg) were mixed at room temperature. The reaction mixture was stirred at room temperature overnight. The solid was filtered off and the solvent was evaporated under reduced pressure. Toluene (20 ml) was added and distilled. The product (4a) (1.0 g, yield 99%) is pure by NMR and used directly for the next step. ¹H NMR (CDCl3): δ 4.58 (s, 1H), 4.22 (t, 2H), 3.74 (t, 2H), 3.67 (t, 2H), 3.40 (s, 3H), 1.91 (s, 3H).

### Synthesis of methyl di(ethylene glycol) 2-(2-nitrobenzylidene) acetoacetate (5a)

Methyl di(ethylene glycol)acetoacetate (3a) (1.02 g, 5.0 mmol) and 2-nitrobenzylaldehyde (811 mg, 5.4 mmol) were dissolved in isopropyl alcohol (3 mL). Then, a mixture of dimethylamine (96.9 mg) and acetic acid (12.36 mg) was added. The reaction solution was stirred at 40° C. overnight. The solvent was evaporated by reduced pressure. The residue was subjected to flash chromatography (ethyl acetate/hexanes=50%˜75%) to obtain the product (5a) as a mixture of geometric isomers (1.34 g, yield 80%). ¹H NMR (CDCl3): δ 8.25-8.23 (m, 1H), 8.22 (s, 1H), 7.60 (d, 0.7H), 7.47 (d, 0.3H), 4.45 (t, 0.6H), 4.21 (t, 1.4H), 3.68-3.40 (m, 6H), 3.36 (s, 3H), 2.50 (s, 3H).

### Synthesis of 2,6-dimethyl 4-(2-nitrophenyl)-1,4-dihydropyridine-3,5-dicarboxylic acid dimethoxy di(ethylene glycol)ester (6a)

Methyl di(ethylene glycol) 3-aminocrotonate (4a) (203 mg, 1.0 mmol) and methyl di(ethylene glycol) 2-(2-nitrobenzylidene)acetoacetate (5a) (337 mg, 1.0 mmol) were dissolved in methanol (5 ml). The reaction was heated to reflux for 30 hours. The solvent was evaporated and the residue was subjected to flash chromatography (acetone/ethyl acetate=2%˜4%) to obtain compound (6a) (309 mg, yield 56%). ¹H NMR (CDCl3): δ 7.72 (d, 1H), 7.51-7.45 (m, 2H), 7.26 (d, 1H), 5.85 (s, 1H), 5.68 (s, 1H), 4.27-4.25 (m, 2H), 4.10-4.07 (m, 2H), 3.662-3.52 (m, 20H), 3.38 (s, 6H), 2.32 (s, 6H). LC/MS: 523 [M+H]⁺, 540 [M+NH₄]⁺, 545 [M+Na]⁺, 561 [M+K]⁺.

### Synthesis of butyl di(ethylene glycol)acetoacetate (3b)

Di(ethylene glycol)butyl ether (16.2 g, 100 mmol) and ethyl acetoacetate (19.5 g, 150 mmol) were heated to 180° C. for three hours and then ethanol and the excess ethyl acetoacetate were distilled out at 160° C. by reduced pressure distillation. The product (3b) (22.1 g, yield 90%) is pure by NMR and used directly for the next step. ¹H NMR (CDCl3): δ 4.23 (t, 2H), 3.65 (t, 2H), 3.56 (t, 2H), 3.51 (t, 2H), 3.40 (s, 2H), 3.38 (t, 2H), 2.20 (s, 3H), 1.46 (m, 2H), 1.30 (m, 2H), 0.84 (t, 3H).

### Synthesis of butyl di(ethylene glycol) 3-aminocrotonate (4b)

Butyl di(ethylene glycol)acetoacetate (3b) (1.23 g, 5.0 mmol) and ammonium hydroxide (0.78 ml, 6.0 mmol), and silica gel powder (60 mg) were mixed at room temperature. The reaction mixture was stirred at room temperature overnight. The solid was filtered off and the solvent was evaporated under reduced pressure. Toluene (20 ml) was added and distilled. The product (1.22 g, yield 99%) is pure by NMR and used directly for the next step. ¹H NMR (CDCl3): δ 4.58 (s, 1H), 4.23 (t, 2H), 3.74-3.59 (m, 6H), 3.49-3.45 (m, 2H), 1.92 (s, 3H), 1.58 (s, 2H), 1.35 (m, 2H), 0.93 (m, 3H).

### Synthesis of methyl di(ethylene glycol) 2-(2-nitrobenzylidene) acetoacetate (5b)

Butyl di(ethylene glycol)acetoacetate (3b) (1.23 g, 5.0 mmol) and 2-nitrobenzylaldehyde (811 mg, 5.4 mmol) were dissolved in isopropyl alcohol (3 mL). Then, a mixture of dimethylamine (96.9 mg) and acetic acid (12.36 mg) was added. The reaction solution was stirred at 40° C. overnight. The solvent was evaporated by reduced pressure. The residue was subjected to flash chromatography (ethyl acetate/hexanes=25%˜40%) to obtain the product (5b) as a mixture of geometric isomers (1.42 g, yield 75%). ¹H NMR (CDCl3): δ 8.25-8.23 (m, 1H), 8.22 (s, 1H), 7.69-7.51 (m, 2H), 7.48 (d, 0.7H), 7.28 (d, 0.3H), 4.45 (t, 0.6H), 4.21 (t, 1.4H), 3.82-3.60 (m, 6H), 3.42 (t, 2H), 2.50 (s, 3H), 1.54 (m, 2H), 1.35 (m, 2H), 0.91 (m, 3H).

### Synthesis of 2,6-dimethyl 4-(2-nitrophenyl)-1,4-dihydropyridine-3,5-dicarboxylic acid dibutoxy di(ethylene glycol)ester (6b)

Butyl di(ethylene glycol) 3-aminocrotonate (4b) (245 mg, 1.0 mmol) and butyl di(ethylene glycol) 2-(2-nitrobenzylidene)acetoacetate (5b) (379 mg, 1.0 mmol) were dissolved in methanol (5 ml). The reaction was heated to reflux for three days. The solvent was evaporated and the residue was subjected to flash chromatography (ethyl acetate/hexanes=25%˜40%) to obtain compound (6b) (250 mg, yield 41%). ¹H NMR (CDCl3) δ 7.72 (d, 1H), 7.52-7.45 (m, 2H), 7.25 (d, 1H), 5.85 (s, 1H), 5.67 (s, 1H), 4.26-4.08 (m, 2H), 4.10-4.06 (m, 2H), 3.67-3.55 (m, 12H), 3.44 (t, 4H), 2.33 (s, 6H), 1.56 (m, 4H), 1.35 (m, 4H), 0.92 (t, 6H). LC/MS: 624 [M+NH₄]⁺.

### Synthesis of hexyl di(ethylene glycol)acetoacetate (3c)

Di(ethylene glycol) hexyl ether (19.0 g, 100 mmol) and ethyl acetoacetate (19.5 g, 150 mmol) were heated to 180° C. for three hours and then ethanol and the excess ethyl acetoacetate were distilled out at 160° C. by reduced pressure distillation. The product (3c) (24.1 g, yield 90%) is pure by NMR and used directly for the next step. ¹H NMR (CDCl3): δ 4.30 (t, 2H), 3.72 (t, 2H), 3.64 (t, 2H), 3.61 (t, 2H), 3.48 (s, 2H), 3.44 (t, 2H), 2.27 (s, 3H), 1.56 (m, 2H), 1.29 (m, 6H), 0.88 (t, 3H).

### Synthesis of hexyl di(ethylene glycol) 3-aminocrotonate (4c)

Hexyl di(ethylene glycol)acetoacetate (3c) (1.37 g, 5.0 mmol) and ammonium hydroxide (0.78 ml, 6.0 mmol), and silica gel powder (60 mg) were mixed at room temperature. The reaction mixture was stirred at room temperature overnight. The solid was filtered off and the solvent was evaporated under reduced pressure. Toluene (20 ml) was added and distilled. The product (1.36 g, yield 99%) is pure by NMR and used directly for the next step. ¹H NMR (CDCl3): δ 4.59 (s, 1H), 4.23 (t, 2H), 3.75-3.47 (m, 6H), 3.49-3.45 (m, 2H), 1.92 (s, 3H), 1.62 (s, 2H), 1.31 (m, 6H), 0.90 (t, 3H).

### Synthesis of hexyl di(ethylene glycol) 2-(2-nitrobenzylidene)acetoacetate (5c)

Hexyl di(ethylene glycol)acetoacetate (3c) (1.37 g, 5.0 mmol) and 2-nitrobenzylaldehyde (811 mg, 5.4 mmol) were dissolved in IPA (3 mL). Then a mixture of dimethylamine (96.9 mg) and acetic acid (12.36 mg) was added. The reaction solution was stirred at 40° C. overnight. The solvent was evaporated by reduced pressure. The residue was subjected to flash chromatography (ethyl acetate/hexanes=25%˜40%) to obtain the product (5c) as a mixture of geometric isomers (1.42 g, yield 75%). ¹H NMR (CDCl3): δ 8.26-8.23 (m, 1H), 7.69-7.61 (m, 2H), 7.56 (d, 0.7H), 7.28 (d, 0.3H), 4.46 (t, 0.6H), 4.21 (t, 1.4H), 3.83-3.41 (m, 6H), 3.42 (t, 3H), 2.51 (s, 3H), 1.57 (m, 2H), 1.31 (m, 6H), 0.90 (t, 3H).

### Synthesis of 2,6-dimethyl 4-(2-nitrophenyl)-1,4-dihydropyridine-3,5-dicarboxylic acid dihexoxy di(ethylene glycol)Ester (6c)

Hexyl di(ethylene glycol) 3-aminocrotonate (4c) (273 mg, 1.0 mmol) and hexyl di(ethylene glycol) 2-(2-nitrobenzylidene)acetoacetate (5c) (407 mg, 1.0 mmol) were dissolved in methanol (5 ml). The reaction was heated to reflux for three days. The solvent was evaporated and the residue was subjected to flash chromatography (ethyl acetate/hexanes=25%˜40%) to obtain compound (6c) (220 mg, yield 33%). ¹H NMR (CDCl3): δ 7.73 (d, 1H), 7.54-7.45 (m, 2H), 7.25 (m, 1H), 5.85 (s, 1H), 5.72 (s, 1H), 4.28-4.07 (m, 2H), 4.12-4.05 (m, 2H), 3.67-3.43 (m, 12H), 3.40 (t, 4H), 2.32 (s, 6H), 1.63-1.52 (m, 4H), 1.37-1.29 (m, 12H), 0.89 (t, 6H). LC/MS: 680 [M+NH₄]⁺, 685 [M+Na]⁺.

### Example 4

### De Novo Synthesis of PEC-Verapamil

### Approach A

PEG-Verapamil was prepared using a first approach. Schematically, the approach followed for this example is shown below (compound numbers in bold in the schematic correspond to the compound numbers provided in the text of this Example 4 alone).

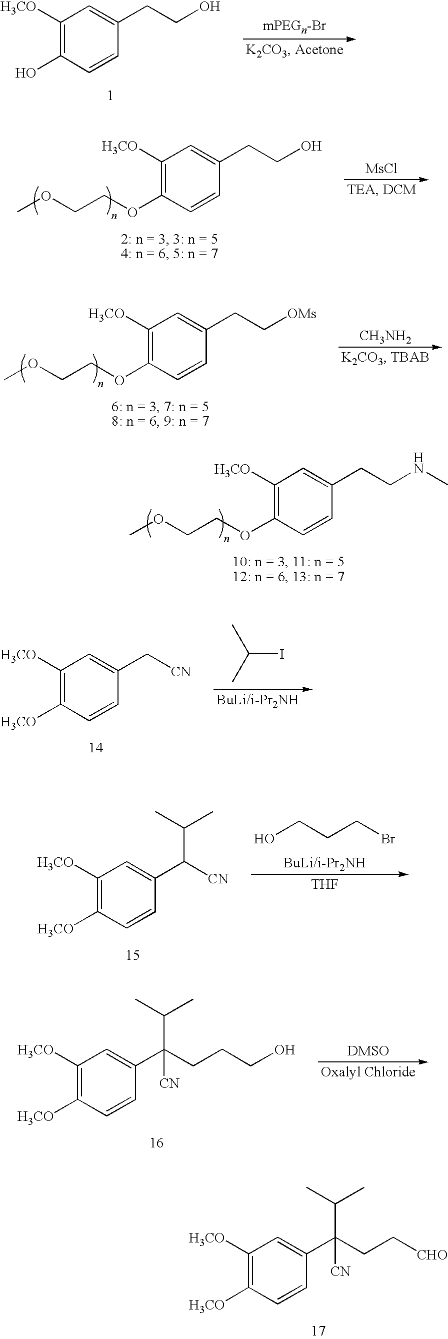

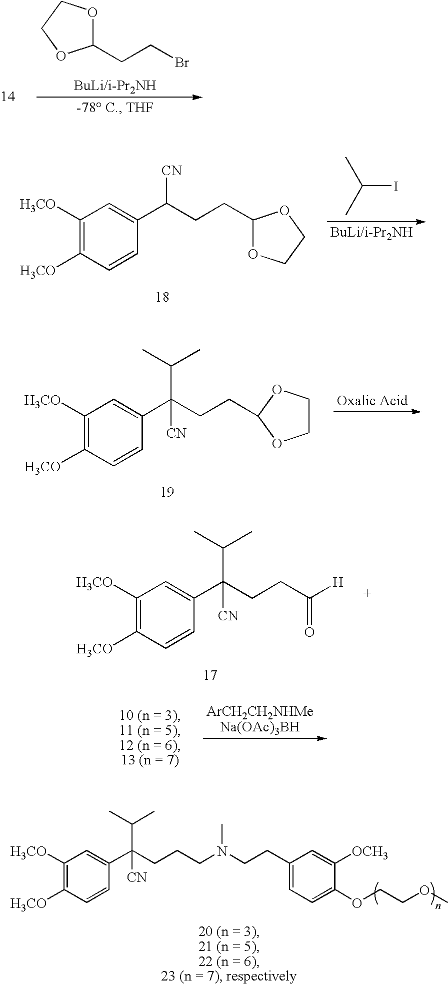

In carrying out this synthesis, the following materials were used. Homovanillyl alcohol, Aldrich, 99%, Cat. No. 148830-10G, Batch No. 19516EO; potassium carbonate, Aldrich, 99%; triethylamine, Aldrich, 99.5%, Cat. No. 471283-500 mL, Batch No. 04623HD; methanesulfonyl chloride, Aldrich, 99.5%, Cat. No. 471259-500 mL, Batch No. 13209KC; 3.4-dimethoxyphenylacetonitrile, Aldrich, Cat. No. 126349-100G, Batch No. 08011BD; 2-iodopropane, Aldrich, 99%, Cat. No. 148938-100G, Batch #03604DD; diisopropylamine, redistilled, Aldrich, 99.95%, Cat. No. 386464-100 mL, Batch #00944TD; butyllithium, 1.6 M solution in hexanes, Aldrich, Cat. No. 186171-100 mL, Batch #20709PD; 3-bromo-1-propanol, Aldrich, 97%, Cat. No. 167169-25G, Batch No. 0901DE; dichloromethane, Aldrich, Cat. No. 270997-2L, Batch #: 00434KD; sodium triacetoxyborohydride, Aldrich, 95%, Cat. No. 316393-25G, Batch No. 07920LD; N, N-diisopropylethylamine, Aldrich, Cat. No. 387649-100 mL, Batch No. 06448PC.

### Synthesis of mPEG₅-homovanillyl Alcohol (3)

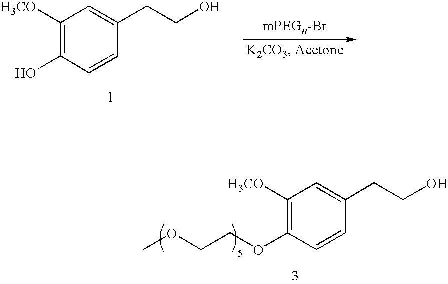

A mixture of homovanillyl alcohol (1) (2.263 g, 13.32 mmol) and mPEG₅-Br (5.1 g, 16.18 mmol) in acetone (50 mL) in the presence of potassium carbonate (9.28 g, 66.47 mmol) was heated to reflux for 21 hours. Based on the results of HPLC, more of mPEG₅-Br (0.9327 g, 2.96 mmol) was added. The mixture was heated to reflux for another 23 hours. The reaction mixture cooled to room temperature, filtered and washed with acetone. The solvent was removed under reduced pressure to afford the crude product (3). Based on the ¹H-NMR, some mPEG₅-Br was contained in the crude mixture. The mixture was used without purification for the next step. ¹H-NMR (CDCl₃): δ 6.86-6.83 (m, 1H), 6.73-6.70 (m, 2H), 4.14 (t, J=4.8-5.7 Hz, 2H), 3.86-3.80 (m, 7H), 3.72-3.69 (m, 2H), 3.66-3.58 (m, 12H), 3.54-3.50 (m, 2H), 3.35 (s, 3H), 2.79 (t, J=6.3-6.6 Hz, 2H). LC-MS: 403.3 (MH⁺), 425.3 (MNa⁺).

### Synthesis of mPEG₅-homovanillyl mesylate (7)

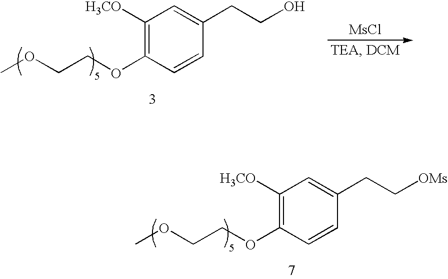

Triethylamine (4.0 ml, 28.55 mmol) was added to a stirred solution of the above crude mPEG₅-homovanillyl alcohol (3) in DCM (40 mL) at room temperature. Methanesulfonyl chloride (1.7 ml, 21.77 mmol) was then added. The resulting mixture was stirred at room temperature for 19 hours. Water was added to quench the reaction. The organic phase was separated and the aqueous phase was extracted with dichloromethane (2×30 mL). The combined organic solution was washed with brine, dried over Na₂SO₄, concentrated to afford yellow oil as the product (7). ¹H-NMR (CDCl₃): δ 6.86-6.83 (m, 1H), 6.74-6.72 (m, 2H), 4.37 (t, J=6.9-7.2 Hz, 2H), 4.14 (t, J=5.1-5.4 Hz, 2H), 3.85 (t, J=5.1 Hz, 2H), 3.83 (s, 3H), 3.73-3.69 (m, 2H), 3.67-3.59 (m, 12H), 3.55-3.52 (m, 2H), 3.36 (s, 3H), 2.98 (t, J=6.9-7.2 Hz, 2H), 2.85 (s, 3H). LC-MS: 481.4 (MH⁺), 503.4 (MNa⁺).

### Synthesis of mPEG₅-homovanillyl methylamine (11)

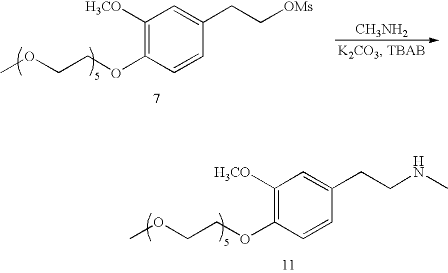

A mixture of the above mPEG₅-homovanillyl mesylate (7) (˜13.32 mmol), potassium carbonate (9.8678 g, 70.68 mmol) and tetrabutylammonium bromide (530 mg, 1.63 mmol) in 33 mL of methylamine solution (2.0 M in THF, 66 mmol) was stirred for 73.5 hours at room temperature. Water was added to quench the reaction and the mixture was concentrated to remove the organic solvents under reduced pressure. The aqueous solution was extracted with DCM (3×40 mL). The combined organic solution was washed with brine, dried over sodium sulfate, concentrated. The residue was purified by flash column chromatography on silica gel using MeOH/DCM (0-10%) and TEA/MeOH/DCM (1/1/9) to afford an oil as the product (11). ¹H-NMR (CDCl₃): δ 6.84-6.81 (m, 1H), 6.72-6.69 (m, 2H), 4.14 (t, J=4.8-5.4 Hz, 2H), 3.84 (t, J=5.4 Hz, 2H), 3.82 (s, 3H), 3.72-3.70 (m, 2H), 3.66-3.59 (m, 12H), 3.55-3.51 (m, 2H), 3.36 (s, 3H), 2.85-2.73 (m, 4H), 2.44 (s, 3H). LC-MS: 416.4 (MH⁺), 438.4 (MNa⁺).

### Synthesis of mPEG₇-homovanillyl methylamine (13)

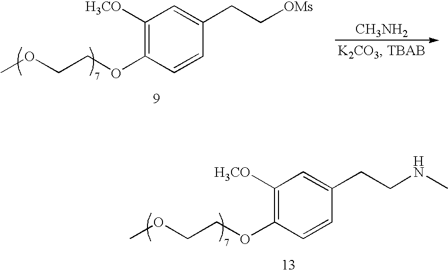

Methylamine (2.0 M solution in THF, 26 ml, 52 mmol) was added to a stirred mixture of crude mPEG₇-homovanillyl mesylate (9) (12.14 mmol) (previously prepared in a manner similar to compound (7) with the exception that mPEG₇-Br is used in place of mPEG₅-Br), potassium carbonate (8.616 g, 61.72 mmol) and tetrabutylammonium bromide (400 mg, 1.23 mmol) were added. After stirring for 24 hours, THF (15 mL) and more of methylamine solution (2.0 M solution in THF, 5.5 mL, 18 mmol) were added. The reaction mixture was stirred at room temperature for 49 hours, water was added and the mixture was concentrated to remove the organic solvents under reduced pressure. The aqueous solution was extracted with DCM (3×60 mL). The combined organic solution was washed with brine (2×100 mL), dried over sodium sulfate, concentrated. The residue was purified by flash column chromatography on silica gel using MeOH/DCM (0-10%) and TEA/MeOH/DCM (0.5/1/9) to afford an oil as the product. ¹H-NMR (CDCl₃): δ 6.84-6.82 (m, 1H), 6.72-6.70 (m, 2H), 4.14 (t, J=5.1-5.4 Hz, 2H), 3.84 (t, J=5.4 Hz, 2H), 3.82 (s, 3H), 3.73-3.69 (m, 2H), 3.66-3.61 (m, 20H), 3.55-3.51 (m, 2H), 3.36 (s, 3H), 2.89-2.75 (m, 4H), 2.46 (s, 3H).

### Synthesis of 2-(3,4-dimethoxy)-5-hydroxy-2-isopropyl-pentanenitrile (16)

A solution of butyllithium (1.6 M solution in hexanes, 2.5 mL, 4.0 mmol) was added via syringe to a solution of diisopropylamine (0.53 mL, 3.75 mmol) in anhydrous THF (18 mL) at −78° C. After five minutes, a solution of 2-(3,4-dimethoxyphenyl)-2-isopropylacetonitrile (15) (273 mg, 1.25 mmol) (previously prepared by reacting 2-iodopropane with 2-(3,4-dimethoxyphenyl)-acetonitrile 14 in a solution diisopropylamine to which butyllithium was added) in THF (3 mL) was added via syringe. The mixture was stirred at −78° C. for ten minutes and then 3-brom-1-propanol (246 mg, 1.70 mmol) was added. The mixture was stirred for 21 hours. During this period, the temperature was changed from −78° C. to room temperature. Saturated NH₄Cl solution (5 mL) was added to quench the reaction and extracted with ether (3×20 mL). After washing with brine and drying with sodium sulfate, the solvent was removed under reduced pressure and the residue was purified by flash column chromatography on SiO₂ using EtOAc/hexanes (0-30%) to afford the product (16) (111.7 mg) in 34% yield. ¹H-NMR (CDCl₃): δ 6.90-6.86 (m, 1H), 6.81-6.79 (m, 1H), 3.84 (s, 3H), 3.83 (s, 3H), 3.53 (m, 2H), 2.24-2.17 (m, 1H), 2.08-1.99 (m, 1H), 1.92-1.82 (m, 1H), 1.60-1.50 (m, 1H), 1.25-1.87 (m, 1H), 1.14 (d, J=6.9 Hz, 3H), 0.75 (d, J=6.6 Hz, 3H). LC-MS: 278.1 (MH⁺), 295.1 (M+H₂O)⁺, 300.2 (MNa⁺).

A second run, following a similar procedure, was performed. Briefly, a solution of butyllithium (1.6 M solution in hexanes, 8.0 mL, 12.80 mmol) was added via syringe to a solution of diisopropylamine (1.8 mL, 12.73 mmol) in anhydrous THF (8 mL) at −78° C. After five minutes, a solution of 2-(3,4-dimethoxyphenyl)-2-isopropylacetonitrile (15) (1.145 g, 5.22 mmol) (previously prepared by reacting 2-iodopropane with 2-(3,4-dimethoxyphenyl)-acetonitrile 14 in a solution diisopropylamine to which butyllithium was added) in THF (5 mL) was added via syringe and then followed by an addition of 3-brom-1-propanol (0.45 mL, 4.99 mmol). The resulting solution was stirred at −78° C. for 4 hours, at room temperature for 23.5 hours. Saturated NH₄Cl solution (10 mL) was added to quench the reaction and extracted with ether (3×50 mL). After washing with brine, drying with sodium sulfate, the solvent was removed under reduced pressure and the residue was purified by flash column chromatography on SiO₂ using EtOAc/hexanes (0-50%) to afford the product (16) (1.2239 g) in 85% yield.

### Synthesis of 2-(3,4-dimethoxyphenyl)-2-isopropyl-5-oxopentanenitrile (17)

DMSO (0.07 mL, 0.987 mmol) in dichloromethane (3 mL) was added to a solution of oxalyl chloride (2.0 M solution in dichloromethane, 0.3 mL, 0.6 mmol) in dichloromethane (5 mL) at −78° C. The solution was stirred at −78° C. for 3 minutes and 2-(3,4-dimethoxy)-5-hydroxy-2-isopropyl-pentanenitrile (16) (110 mg, 0.397 mmol) in dichloromethane (3.5 mL) was added. The mixture was stirred at −78° C. for ten minutes and triethylamine (0.5 mL) was added. The resulting reaction mixture was stirred at −78° C. for three hours, and then the dry ice-acetone bath was removed, the mixture was warmed up to room temperature. The reaction mixture was stirred at room temperature for 2.5 hours. Saturated sodium chloride solution (5 mL) was added to quench the reaction. The organic solution was separated and the aqueous solution was extracted with dichloromethane (3×20 mL). The combined organic solution was washed with brine (60 mL), dried over sodium sulfate, concentrated to afford the crude product (109 mg), which was used in the next reaction without further purification. Based on the results of HPLC, the purity of the product was over 96%. ¹H-NMR (CDCl₃): δ 9.68 (s, 1H), 6.92-6.80 (m, 3H), 3.874 (s, 3H), 3.869 (s, 3H), 2.67-2.55 (m, 1H), 2.48-2.39 (m, 1H), 2.23-2.03 (m, 3H), 1.16 (d, J=6.9 Hz, 3H), 0.79 (d, J=6.6 Hz, 3H). LC-MS: 276.2 (MH⁺), 293.2 (M+H₂O)⁺, 298.2 (MNa⁺).

A second run, following the same procedure, was followed, using the alcohol (16) (1.2239 g, 4.413 mmol), DMSO (1.0 mL, 14.10 mmol), oxalyl chloride (2.0 M solution in dichloromethane, 7.0 mL, 14.0 mmol), triethylamine (4 mL) and dichloromethane (28 mL). The crude product was 1.537 g.

### Synthesis of 2-cyano-2-(3,4-dimethoxyphenyl)-2-isopropylethyl-1,3-dioxolane (19)

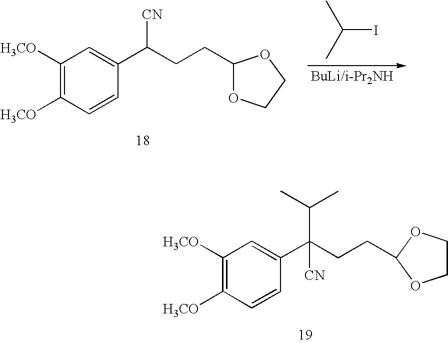

A solution of butyllithium (1.6 M solution in hexanes, 7.0 mL, 11.2 mmol) was added via syringe to a solution of diisopropylamine (1.5 mL, 10.61 mmol) in anhydrous THF (25 mL) at −78° C. And then a solution of 2-(3,4-dimethoxyphenyl)-4-(1,3)-dioxolan-2-yl-butyronitrile (18) (709 mg, 2.56 mmol) (previously prepared by reacting 2-(2-bromoethyl)-1,3-dioxolane with 2-(3,4-dimethoxyphenyl)-acetonitrile 14 in a solution diisopropylamine to which butyllithium was added) in THF (10 mL) was added via syringe. The mixture was stirred at −78° C. for five minutes and then 2-iodopropane (0.4 mL, 3.96 mmol) was added. The mixture was stirred at −78° C. for five hours and then at room temperature for 17.5 hours. Saturated NH₄Cl solution (10 mL) was added to quench the reaction. Ethyl ether (60 mL) was added and the etheral solution was isolated. The aqueous solution was extracted with ether (2×20 mL). After washing with brine, drying with sodium sulfate, the solvent was removed under reduced pressure and the residue was purified by flash column chromatography on SiO₂ using EtOAc/hexanes (0-30%) to afford the product (19) (397 mg) in 49% yield. ¹H-NMR (CDCl₃): δ 6.86-6.82 (m, 3H), 4.80 (t, J=4.2-4.8 Hz, 1H), 3.95-3.77 (m, 4H), 3.878 (s, 3H), 3.867 (s, 3H), 2.24 (dt, J=3.9-4.2 Hz, J=12.9-13.2 Hz, 1H), 2.11-2.02 (m, 1H), 1.91 (dt, J=3.9 Hz, J=12.6 Hz, 1H), 1.72 (tt, J=3.9-4.2 Hz, J=12.6-13.2 Hz, 1H), 1.37-1.24 (m, 1H), 1.16 (d, J=6.6 Hz, 3H), 0.80 (d, J=6.9 Hz, 3H). LC-MS: 342.083 (MNa⁺).

### Synthesis of 2-(3,4-dimethoxyphenyl)-2-isopropyl-5-oxopentanenitrile (17)

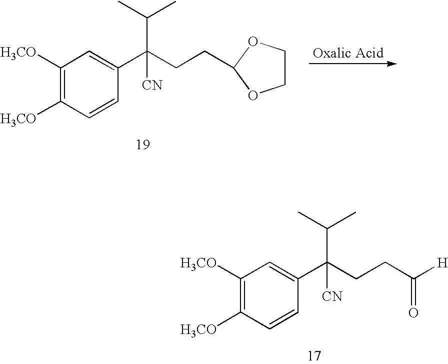

Oxalic acid dehydrate (504.6 mg, 3.96 mmol) was added to a solution of the acetal (19) (372 mg, 1.16 mmol) in acetone (10 mL) and water (10 mL). The resulting mixture was stirred at 80° C. for four hours. The reaction mixture was cooled to room temperature. Potassium carbonate (1.3 g) was added to quench the reaction. The mixture was extracted with ethyl ether (3×20 mL). The organic solution was washed with brine, dried over sodium sulfate, concentrated to afford the crude product (17) (293 mg), which was used in the next step without further purification. The product was confirmed by ¹H-NMR spectra.

### Synthesis of O-mPEG₃-verapamil (20)

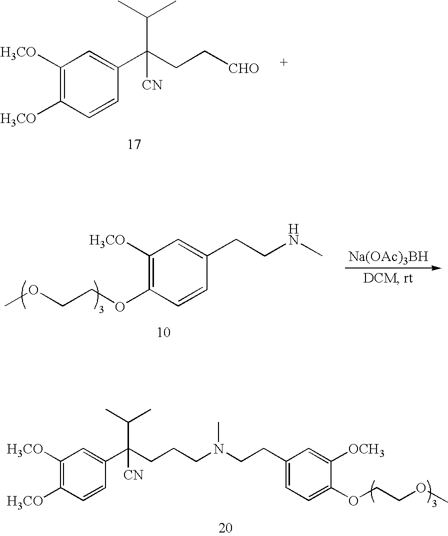

Sodium triacetoxyborohydride (178.6 mg, 0.801 mmol) was added to a stirred solution of 2-(3,4-dimethoxyphenyl)-2-isopropyl-5-oxo-pentanenitrile (17) (106 mg, 0.385 mmol) and mPEG₃-homovanillyl methylamine (10) (134 mg, 0.409 mmol) in dichloromethane (6 mL) at room temperature. The resulting reaction mixture was stirred at room temperature for three hours. Water was added to quench the reaction. The organic solution was separated and the aqueous solution was extracted with dichloromethane (2×20 mL). The combined organic solution was washed with brine, dried over sodium sulfate, concentrated. The residue was purified by flash column chromatography on SiO₂ using EtOAC/hexanes (0-100%) and MeOH/Et₃N/EtOAc (1/1/9) to afford the product (134 mg, 59% yield), with 2-(3,4-dimethoxy)-5-hydroxy-2-isopropyl-pentanenitrile (16) (27 mg, 26% yield). ¹H-NMR of (20) (CDCl₃): δ 6.88-6.77 (m, 4H), 6.64-6.60 (m, 2H), 4.10 (t, J=4.8-5.4 Hz, 2H), 3.84-3.79 (m, 11H), 3.70-3.67 (m, 2H), 3.64-3.59 (m, 4H), 3.51-3.48 (m, 2H), 3.33 (s, 3H), 2.63-2.80 (m, 2H), 2.49-2.42 (m, 2H), 2.38-2.23 (m, 2H), 2.13 (s, 3H), 2.10-1.97 (m, 2H), 1.79 (dt, J=4.2 Hz, J=12.3 Hz, 2H), 1.57-1.45 (m, 1H), 1.50-1.05 (m, 1H), 1.14 (d, J=6.6 Hz, 3H), 0.74 (d, J=6.6 Hz, 3H). LC-MS: 357.4 (MH⁺).

### Synthesis of O-mPEG₅-verapamil (21)

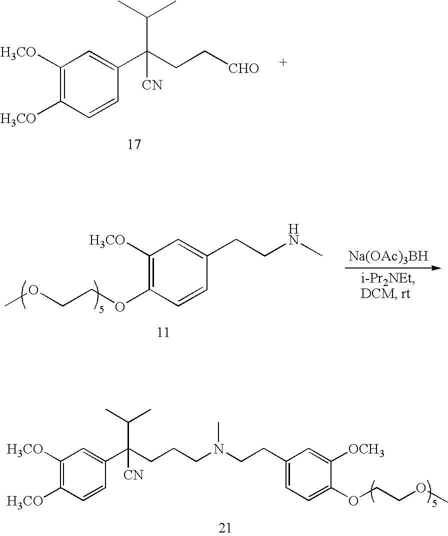

A mixture of mPEG₅-homovanillyl methylamine (11) (123 mg, 0.447 mmol) and 2-(3,4-dimethoxyphenyl)-2-isopropyl-5-oxo-pentanenitrile (17) (210 mg, 0.505 mmol) was stirred for ten minutes and then i-Pr₂NEt (0.02 mL) was added. After ten minutes at room temperature, sodium triacetoxyborohydride (138 mg, 0.619 mmol) was added. After 25 minutes, more of Na(OAc)₃BH (90 mg, 0.403 mmol) was added. The resulting reaction mixture was stirred at room temperature for 5.5 hours. Water was added to quench the reaction. The organic solution was separated and the aqueous solution was extracted with dichloromethane (2×20 mL). The combined organic solution was washed with brine (60 mL), dried over sodium sulfate, concentrated. The residue was purified by flash column chromatography on SiO₂ using EtOAC/hexanes (30-100%) and Et₃N/EtOAc (1/20) to afford the product (125 mg, 42% yield). ¹H-NMR (CDCl₃): δ 6.84-6.79 (m, 4H), 6.66-6.62 (m, 2H), 4.12 (t, J=4.8-5.1 Hz, 2H), 3.86-3.81 (m, 11H), 3.72-3.68 (m, 2H), 3.66-3.59 (m, 12H), 3.55-3.51 (m, 2H), 3.35 (s, 3H), 2.65-2.61 (m, 2H), 2.49-2.44 (m, 2H), 2.37-2.29 (m, 2H), 2.15 (s, 3H), 2.13-1.99 (m, 2H), 1.81 (dt, J=4.2 Hz, J=12.3 Hz, 2H), 1.54 (m, 1H), 1.16 (d, J=6.6 Hz, 3H), 1.12 (m, 1H), 0.77 (d, J=6.6 Hz, 3H). LC-MS: 675.5 (MH⁺), 697.5 (MNa⁺).

### Synthesis of O-mPEG₆-verapamil (22)

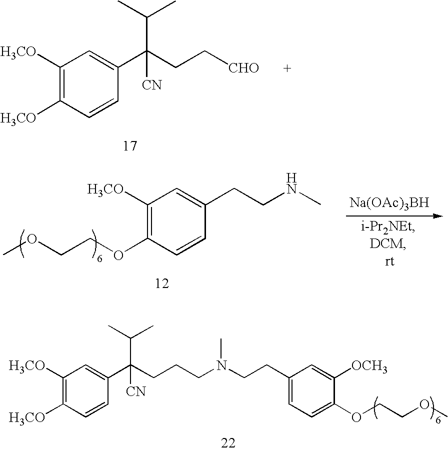

A mixture of mPEG₆-homovanillyl methylamine (12) (306 mg, 0.67 mmol) and 2-(3,4-dimethoxyphenyl)-2-isopropyl-5-oxo-pentanenitrile (17) (170 mg, 0.62 mmol) was stirred for 5 minutes and then i-Pr₂NEt (0.03 mL, 0.17 mmol) was added. After five minutes at room temperature, sodium triacetoxyborohydride (296 mg, 1.33 mmol) was added. The resulting reaction mixture was stirred at room temperature for 23 hours. Water was added to quench the reaction. The organic solution was separated and the aqueous solution was extracted with dichloromethane (2×30 mL). The combined organic solution was washed with brine (60 mL), dried over sodium sulfate, concentrated. The residue was purified by flash column chromatography on SiO₂ to afford the product (195 mg, 44% yield). ¹H-NMR (CDCl₃): δ 6.82-6.77 (m, 4H), 6.65-6.60 (m, 2H), 4.10 (t, J=5.1-5.4 Hz, 2H), 3.84-3.79 (m, 11H), 3.70-3.66 (m, 2H), 3.64-3.57 (m, 16H), 3.53-3.49 (m, 2H), 3.33 (s, 3H), 2.65-2.61 (m, 2H), 2.51-2.46 (m, 2H), 2.39-2.29 (m, 2H), 2.16 (s, 3H), 2.13-1.98 (m, 2H), 1.82 (dt, J=4.2 Hz, J=12.3 Hz, 2H), 1.54-1.48 (m, 1H), 1.14 (d, J=6.6 Hz, 3H), 1.11 (m, 1H), 0.75 (d, J=6.6 Hz, 3H). LC-MS: 719.5 (MH⁺), 741.5 (MNa⁺).

### Synthesis of O-mPEG₇-verapamil (23)

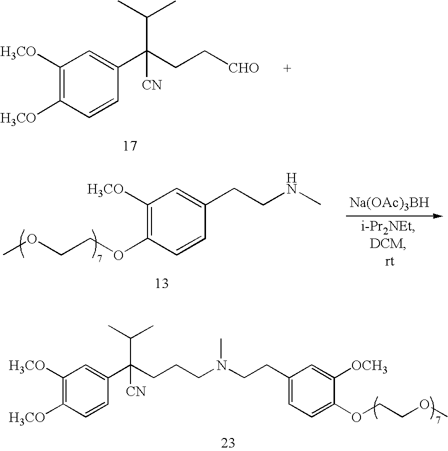

i-Pr₂NEt (0.03 mL) was added to a stirred mixture of mPEG₇-homovanillyl methylamine (13) (290 mg, 0.576 mmol) and 2-(3,4-dimethoxyphenyl)-2-isopropyl-5-oxo-pentanenitrile (17) (167 mg, 0.607 mmol). After five min at room temperature, sodium triacetoxyborohydride (260 mg, 1.104 mmol) was added. The resulting reaction mixture was stirred at room temperature for 5.5 hours. Water was added to quench the reaction. The organic solution was separated and the aqueous solution was extracted with dichloromethane (2×20 mL). The combined organic solution was washed with brine, dried over sodium sulfate, concentrated. The residue was purified by flash column chromatography on SiO₂ using EtOAC/hexanes (30-100%) and Et₃N/MeOH/EtOAc (0.5/1/25) to afford the product (302 mg, 69% yield). ¹H-NMR (CDCl₃): δ 6.83-6.79 (m, 4H), 6.67-6.62 (m, 2H), 4.12 (t, J=5.1-5.4 Hz, 2H), 3.86-3.81 (m, 11H), 3.72-3.68 (m, 2H), 3.66-3.59 (m, 20H), 3.55-3.51 (m, 2H), 3.35 (s, 3H), 2.65-2.60 (m, 2H), 2.49-2.44 (m, 2H), 2.36-2.01 (m, 2H), 2.15 (s, 3H), 2.13-2.01 (m, 2H), 1.81 (dt, J=4.2 Hz, J=12.3 Hz, 2H), 1.53 (m, 1H), 1.16 (d, J=6.6 Hz, 3H), 1.12 (m, 1H), 0.77 (d, J=6.6 Hz, 3H). LC-MS: 763.5.5 (MH⁺), 785.5 (MNa⁺).

### Example 5

### De Novo Synthesis of PEG-Verapamil

### Approach B

PEG-Verapamil was prepared using a second approach. Schematically, the approach followed for this example is shown below (compound numbers in bold in the schematic correspond to the compound numbers provided in the text of this Example 5 alone).

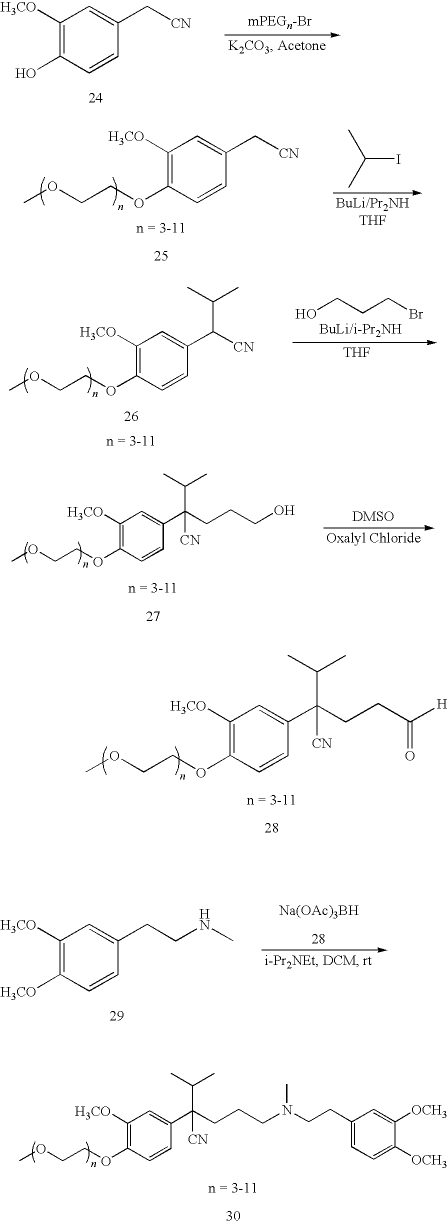

In carrying out this synthesis, the following materials were used. Homovanillyl alcohol, Aldrich, 99%, Cat. No. 148830-10G, Batch No. 19516EO; potassium carbonate, Aldrich, 99%; triethylamine, Aldrich, 99.5%, Cat. No. 471283-500 mL, Batch No. 04623HD; methanesulfonyl chloride, Aldrich, 99.5%, Cat. No. 471259-500 mL, Batch No. 13209KC; 3.4-dimethoxyphenylacetonitrile, Aldrich, Cat. No. 126349-100G, Batch No. 08011BD; 2-iodopropane, Aldrich, 99%, Cat. No. 148938-100G, Batch #, 03604DD; diisopropylamine, redistilled, Aldrich, 99.95%, Cat. No. 386464-100 mL, Batch #00944TD; butyllithium, 1.6 M solution in hexanes, Aldrich, Cat. No. 186171-100 mL, Batch #20709PD; 3-bromo-1-propanol, Aldrich, 97%, Cat. No. 167169-25G, Batch No. 0901DE; dichloromethane, Aldrich, Cat. No. 270997-2L, Batch #: 00434KD; sodium triacetoxyborohydride, Aldrich, 95%, Cat. No. 316393-25G, Batch No. 07920LD; N,N-diisopropylethylamine, Aldrich, Cat. No. 387649-100 mL, Batch No. 06448PC; N-methylhomoveratrylamine, Aldrich, Cat. No. 334774, Batch No. 10421EO.

### Synthesis of 4-mPEG₃-3-methoxyphenylacetonitrile (25)

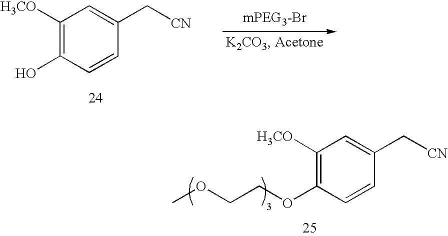

A mixture of 4-hydroxy-3-methoxyphenylacetonitrile (24) (503 mg, 3.05 mmol) and mPEG₃-Br (830 mg, 3.65 mmol, 1.2 eq) in the presence of potassium carbonate (2.35 g, 16.83 mmol) in acetone (15 mL) was heated to reflux for 17 hours. The mixture was cooled to room temperature, filtered and washed with acetone and DCM. The solution was concentrated. The residue was purified by column chromatography on silica gel using MeOH/DCM (0-2%) to afford pure product (25) (738 mg) and a mixture of the product and mPEG₃-Br (277 mg). No attempt was made to further purify the mixture.

A second run, following a similar procedure, was performed. A mixture of 4-hydroxy-3-methoxyphenylacetonitrile (24) (1.81 g, 10.98 mmol) and mPEG₃-Br (2.505 g, 11.03 mmol, 1.005 eq) in the presence of potassium carbonate (6.693 g, 47.94 mmol) in acetone (35 mL) was heated to reflux for 20.5 hours. The mixture was cooled to room temperature, filtered and washed with acetone. The solution was concentrated. The residue was purified by column chromatography on silica gel using EtOAc/hexanes (0-50%) to afford pure product (25) (2.826 g, 83%). Note: Based on the results of HPLC and TLC, a small amount of the starting nitrile material (24) was observed. No mPEG₃-Br was isolated and observed in the NMR spectra. ¹H-NMR (CDCl₃): δ 6.92-6.89 (m, 1H), 6.82-6.79 (m, 2H), 4.16 (t, J=5.1-5.7 Hz, 2H), 3.88-3.85 (m, 5H), 3.74-3.70 (m, 2H), 3.67-3.62 (m, 6H), 3.54-3.51 (m, 2H), 3.36 (s, 3H). LC-MS: 310.2 (MH⁺).

### Synthesis of 2-(3-methoxy-4-mPEG₃-phenyl)-3-methylbutyronitrile (26)

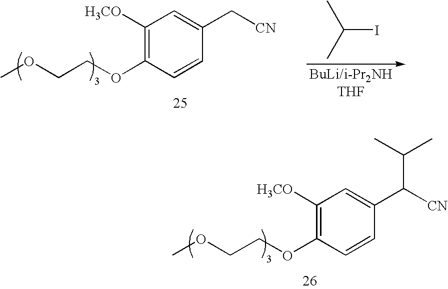

Butyllithium solution (1.6 M in hexanes, 5 mL, 8.0 mmol) was added to a stirred solution of i-Pr₂NH (1.13 mL, 7.99 mmol) in anhydrous THF (10 mL) at −78° C. After five minutes, 4-mPEG₃-3-methoxyphenylacetonitrile (25) (2.450 g, 7.92 mmol) in THF (20 mL) was added, followed by an addition of 2-iodopropane (0.8 mL, 7.92 mmol). The resulting mixture was stirred at −78° C. for five hours. The dry-acetone bath was removed. The reaction mixture was warmed up to room temperature and stirred at room temperature for 16 hours. Saturated NH₄Cl solution was added to quench the reaction. The solution was extracted with ethyl ether (3×20 mL). The combined organic solution was washed with brine, dried over sodium sulfate, concentrated. The residue was separated by column chromatography on silica gel using EtOAc/hexane (0-50%) to afford the product (26) (1.7656 g, 74%), along with 0.3629 g of starting material. ¹H-NMR (CDCl₃): δ 6.88-6.85 (m, 1H), 6.79-6.76 (m, 2H), 4.15 (t, J=4.8-5.4 Hz, 2H), 3.87-3.84 (m, 5H), 3.73-3.70 (m, 2H), 3.66-3.61 (m, 4H), 3.55-3.50 (m, 3H), 3.35 (s, 3H), 2.12-1.77 (m, 1H), 1.01 (d, J=6.6 Hz, 6H). LC-MS: 352.3 (MH⁺).

### Synthesis of 3-hydroxy-2-(3-methoxy-4-mPEG₃-phenyl)-2-isopropyl-1-pentanenitrile (27)

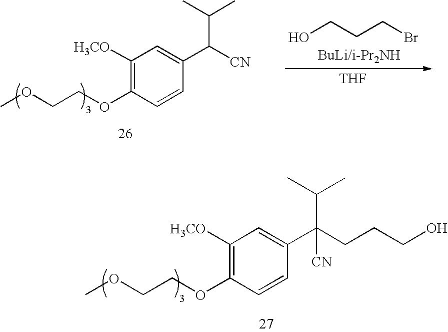

Butyllithium (1.6 M solution in hexanes, 8.0 mL, 12.80 mmol) was added to a solution of diisopropylamine (1.8 mL, 12.73 mmol) in THF (6 mL) at −78° C. Then, a solution of 2-(3-methoxy-4-mPEG₃-phenyl)-3-methylbutyronitrile (26) (1.76 g, 5.01 mmol) in THF (9 mL) was added. The resulting mixture was stirred for ten minutes and 3-bromo-1-propanol (0.55 mL, 6.10 mmol) was added. The resulting mixture was stirred at −78° C. for three hours and then at room temperature for three hours. Saturated NH₄Cl (10 mL) was added to quench the reaction. The mixture was extracted with ethyl ether (4×40 mL). The combined organic solution was washed with brine (100 mL), dried over sodium sulfate, concentrated. The residue was purified by flash column chromatography on silica gel using EtOAc/hexanes (30%, 50% and 100%) to afford 1.73 g of the product (27) in 84% yield. ¹H-NMR (CDCl₃): δ 6.87-6.85 (m, 3H), 4.17 (t, J=4.8-5.4 Hz, 2H), 3.89-3.85 (m, 5H), 3.74-3.71 (m, 2H), 3.67-3.62 (m, 4H), 3.61-3.57 (m, 2H), 3.54-3.51 (m, 2H), 3.36 (s, 3H), 2.24-2.14 (m, 1H), 2.10-2.03 (m, 1H), 1.95-1.85 (m, 1H), 1.64-1.55 (m, 1H), 1.26-1.16 (m, 1H), 1.17 (d, J=6.6 Hz, 2H), 0.78 (d, J=6.6 Hz, 6H). LC-MS: 410.3 (MH⁺), 432.3 (MNa⁺).

### Synthesis of 2-(3-methoxy-4-mPEG₃-phenyl)-2-isopropyl-5-oxo-pentanenitrile (28)

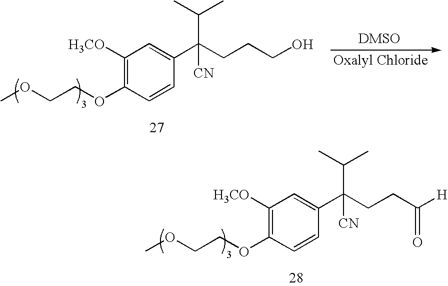

Oxalyl chloride (2.0 m solution in dichloromethane, 5.4 mL, 10.80 mmol) was added to dichloromethane (6 mL) at −78° C. Then a solution of DMSO (4.0 mL, 11.28 mmol) in DCM (4 mL) was added. After about five minutes, a solution of the alcohol (27) (1.415 g, 3.46 mmol) in DCM (10 mL) was added. After 15 minutes at −78° C., triethylamine (3.5 mL) was added. The resulting mixture was stirred for 16.5 hours. During the period, the temperature was allowed to reach room temperature. The bath was removed, and the mixture was stirred at room temperature for another hour. Saturated ammonium chloride was added to quench the reaction, extracted with ethyl ether (3×60 mL). The combined organic solution was washed with brine (2×100 mL), dried over sodium sulfate, concentrated to afford the crude product (28), which was used in the next step without further purification. ¹H-NMR (CDCl₃): δ 9.65 (s, 1H), 6.89-6.80 (m, 3H), 4.16 (t, J=5.1 Hz, 2H), 3.89-3.86 (t, J=5.1 Hz, 2H), 3.85 (s, 3H), 3.74-3.71 (m, 2H), 3.68-3.60 (m, 4H), 3.55-3.52 (m, 2H), 3.36 (s, 3H), 2.66-2.54 (m, 1H), 2.48-2.38 (m, 1H), 2.21-2.03 (m, 3H), 1.41 (m, 1H), 1.2 (d, J=6.6 Hz, 2H), 0.79 (d, J=6.6 Hz, 6H). LC-MS: 408.3 (MH⁺), 430.3 (MNa⁺).

### Synthesis of O-mPEG₃-Verapmil (30)

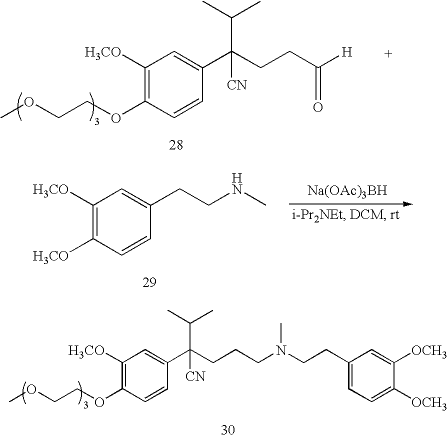

i-Pr₂NEt (0.02 mL, 0.11 mmol) was added to a stirred solution of 2-(3-methoxy-4-mPEG₃-phenyl)-2-isopropyl-5-oxo-pentanenitrile (28) (145 mg, 0.36 mmol) and N-methylhomoveratrylamine (29) (119 mg, 0.59 mmol) in dichloromethane (6 mL). Sodium triacetoxyborohydride (182 mg, 0.82 mmol) was added. The mixture was stirred at room temperature for six hours. Water was added to quench the reaction. The organic solution was separated and the aqueous solution was extracted with dichloromethane (4×15 mL). The combined organic solution was washed with brine, dried over sodium sulfate, concentrated. The residue was purified by flash column chromatography on SiO₂ using EtOAC/hexanes (30-100%) and MeOH/Et₃N/EtOAc (2/1/20) to afford the product (30) (171 mg, 76% yield). The purity was >94% based on HPLC. The product was purified again with preparative TLC and flash column chromatography on silica gel using MeOH/DCM (0-5%) to afford 130 mg of the final product. ¹H-NMR (CDCl₃): δ 6.85-6.84 (m, 3H), 6.78-6.75 (m, 1H), 6.69-6.67 (m, 2H), 4.10 (t, J=4.8-5.4 Hz, 2H), 3.87-3.83 (m, 11H), 3.74-3.71 (m, 2H), 3.68-3.62 (m, 4H), 3.55-3.52 (m, 2H), 3.36 (s, 3H), 2.67-2.62 (m, 2H), 2.51-2.45 (m, 2H), 2.36-2.27 (m, 2H), 2.15 (s, 3H), 2.12-2.00 (m, 2H), 1.85-1.75 (m, 1H), 1.49 (m, 1H), 1.24 (m, 1H), 1.16 (d, J=6.6 Hz, 3H), 0.76 (d, J=6.6 Hz, 3H). LC-MS: 587.4 (MH⁺).

### Example 6

### De Novo Synthesis of PEG-Verapamil

### Approach C

PEG-Verapamil was prepared using a third approach. Schematically, the approach followed for this example is shown below (unless otherwise stated, compound numbers in bold in the schematic correspond to the compound numbers provided in the text of this Example 6 alone).

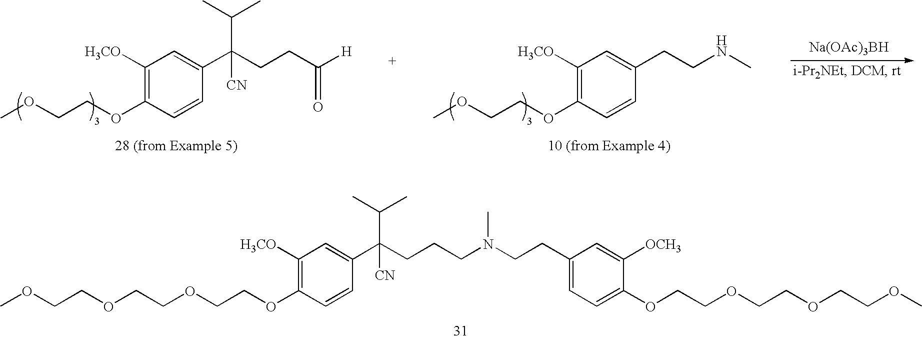

### Synthesis of O,O′-di-mPEG₃-Verapamil (31)

i-Pr₂NEt (0.02 mL, 0.11 mmol) was added to a stirred solution of 2-(3-methoxy-4-mPEG₃-phenyl)-2-isopropyl-5-oxo-pentanenitrile (28) (176 mg, 0.43 mmol) (prepared in accordance with the procedure provided in Example 5) and the mPEG₃ methylamine (10) (148 mg, 0.45 mmol) (prepared in accordance with the procedure provided in Example 4) in dichloromethane (6 mL). Sodium triacetoxyborohydride (225 mg, 1.01 mmol) was added. The mixture was stirred at room temperature for six hours. Water was added to quench the reaction. The organic solution was separated and the aqueous solution was extracted with dichloromethane (4×15 mL). The combined organic solution was washed with brine, dried over sodium sulfate, concentrated. The residue was purified by flash column chromatography on SiO₂ using EtOAC/hexanes (30-100%) and MeOH/EtOAc (0-10%) and TEA/MeOH/EtOAc (1/2/25) to afford 244 mg of the product (31) in 79% yield. ¹H-NMR (CDCl₃): δ 6.85-6.79 (m, 4H), 6.67-6.62 (m, 2H), 4.17-4.11 (m, 4H), 3.88-3.81 (m, 10H), 3.74-3.70 (m, 4H), 3.67-3.61 (m, 8H), 3.55-3.52 (m, 4H), 3.36 (s, 6H), 2.66-2.61 (m, 2H), 2.50-2.45 (m, 2H), 2.33-2.27 (m, 2H), 2.14 (s, 3H), 2.05-2.00 (m, 2H), 1.85-1.75 (m, 1H), 1.49 (m, 1H), 1.22 (m, 1H), 1.15 (d, J=6.6 Hz, 3H), 0.76 (d, J=6.6 Hz, 3H). LC-MS: 719.5 (MH⁺), 741.5 (MNa⁺).

### Example 7

### Calcium Channel Binding Assay

A binding assay was performed similar to that set forth in Example 2, except that ³[H]-diltiazam is used as the competing radioligand. The results are shown below (mPEG₆-O-Verapamil was run separately wherein the diltiazem control had an IC₅₀=3.21×10-7).

TABLE 1
Results from Binding Assay

| Drug | IC₅₀ (M) |
|---|---|
| Diltiazem | 2.89 × 10−8 |
| Verapamil | 2.98 × 10−8 |
| mPEG₃-O-Verapamil | 5.41 × 10−7 |
| (compound 20 from Example 4) | |
| mPEG₅-O-Verapamil | 8.52 × 10−7 |
| (compound 21 from Example 4) | |
| mPEG₆-O-Verapamil | 3.49 × 10−7* |
| (compound 22 from Example 4) | |
| mPEG₇-O-Verapamil | 1.13 × 10−6 |
| (compound 23 from Example 4) | |

### Example 8

### De Novo Synthesis of mPEG-Dantrolene

PEG-Dantrolene was prepared. Schematically, the approach followed for this example is shown below (unless otherwise stated, compound numbers in bold in the schematic correspond to the compound numbers provided in the text of this Example 8 alone).

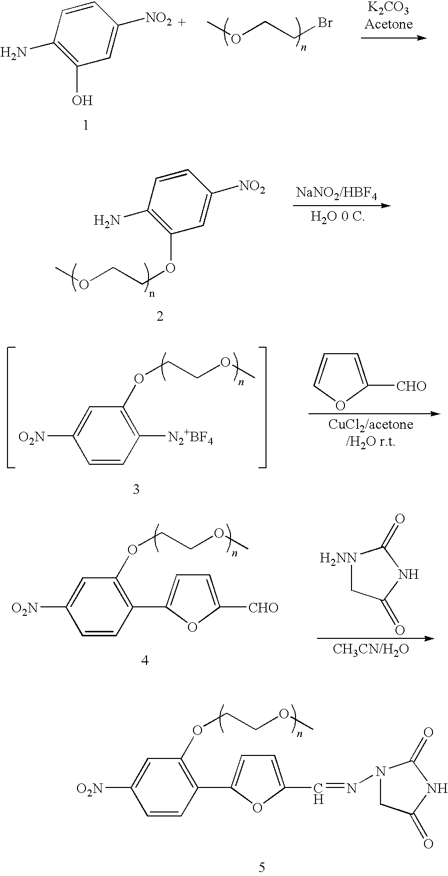

### General Synthesis of mPEGn-O-dantrolene (n=3, 5 and 7)

### Synthesis of Compound (2)

2-Amino-5-nitrophenol (462 mg, 3.0 mmol) and mPEGₙ-Br [n=3, 5, 7, in three separate runs] (3.0 mmol) were dissolved in acetone (25 mL). K₂CO₃ (828 mg, 6.0 mmol) was added to the solution and the reaction was heated at reflux for four hours. The acetone was removed under reduced pressure and the residue was dissolved in DCM (300 mL). The organic phase was washed with H₂O (2×300), dried over Na₂SO₄ and solvent was removed under reduced pressure. The product was used without further purification in the next reaction step. mPEG₃-aminobenzonitrile (2a): ¹H NMR (300 MHz, CDCl₃): δ 7.88 (d. 1H), 7.75 (s, 1H), 7.74 (d, 1H), 4.88 (br, 2H), 4.35 (m, 2H), 3.85 (m, 2H), 3.7 (m, 6H), 3.63 (m, 2H), 3.40 (s, 3H). mPEG₅-aminobenzonitrile (2b): ¹H NMR (300 MHz, CDCl₃): 7.88 (d. 1H), 7.75 (s, 1H), 7.74 (d, 1H), 5.05 (br, 2H), 4.35 (m, 2H), 3.85 (m, 2H), 3.75 (m, 2H), 3.7 (m, 12H), 3.63 (m, 2H), 3.40 (s, 3H). mPEG₇-aminobenzonitrile (2c): ¹H NMR (300 MHz, CDCl₃): δ 7.85 (d. 1H), 7.71 (s, 1H), 6.66 (d, 1H), 5.05 (br, 2H), 4.25 (m, 2H), 3.87 (m, 2H), 3.7-3.3 (m, 22H), 3.63 (m, 2H), 3.38 (s, 3H).

### Synthesis of Compound (4)

Compound (2) (1.0 mmol) [n=3, 5, 7, in three separate runs] was dissolved in water (3 mL), and 48% HBF₄ (0.6 g) was added. The resulting solution was cooled in an ice-bath and then NaNO₂ (85 mg 1.2 mmol) was added dropwise. The mixture was stirred for 30 minutes at 0° C. A solution of 2-furaldehyde (384 mg, 4.0 mmol) in acetone (3 mL) was added followed by the addition of CuCl₂ (16 mg, 0.12 mmol) in water (0.5 mL). The reaction mixture was stirred at room temperature for 16 hours. Dichloromethane (200 mL) was added to the mixture and the organic phase was washed with H₂O (200 ml×3), dried over Na₂SO₄ and the solvent removed under reduced pressure. The product was obtained as red sticky oil which was used in the next reaction step reaction without further purification.

### Synthesis of mPEG-O-dantrolene conjugate (5)

Compound (4) [n=3, 5, 7, in three separate runs] was dissolved in CH₃CN (10 mL), and then 1-aminohydantoin hydrochloride (450 mg, 3 mmol) in water (10 mL) was added. The solution was stirred at room temperature for one hour, and then DCM (200 mL) was added to the stirring solution. The organic phase was washed with H₂O (200 ml×3), dried over Na₂SO₄, and the solvent removed under reduced pressure. The crude product was purified by column chromatography (Biotage flash chromatography system; A: MeOH, 1-4% (20CV), 4-6% (10CV), B: DCM). The product was obtained as yellow solid (overall yield: 40-60%). mPEG₃-O-dantrolene (5a): ¹H NMR (300 MHz, CDCl₃): δ 8.14 (d. 1H), 7.95 (s, 1H), 7.94 (d, 1H), 7.84 (s, 1H), 7.42 (d, 1H), 7.00 (d, 1H), 4.38 (m, 2H), 4.31 (s, 2H), 4.03 (m, 2H), 3.8 (m, 2H), 3.73 (m, 2H), 3.68 (m, 2H), 3.55 (m, 2H), 3.38 (s, 3H). LC-MS: 477.2 (M+H⁺). mPEG₅-O-dantrolene (5b): ¹H NMR (300 MHz, CDCl₃): δ 8.14 (d. 1H), 7.95 (d, 1H), 7.94 (s, 1H), 7.84 (s, 1H), 7.42 (d, 1H), 7.00 (d, 1H), 4.38 (m, 2H), 4.31 (s, 2H), 4.03 (m, 2H), 3.8 (m, 2H), 3.75 (m, 12H), 3.68 (m, 2H), 3.38 (s, 3H). LC-MS: 565.2 (M+H⁺). mPEG₇-O-dantrolene (5c): ¹H NMR (300 MHz, CDCl₃): δ 8.05 (d. 1H), 7.86 (d, 1H), 7.77 (s, 1H), 7.74 (s, 1H), 7.36 (d, 1H), 6.94 (d, 1H), 4.36 (m, 2H), 4.21 (s, 2H), 3.78 (m, 2H), 3.75 (m, 2H), 3.73 (m, 2H), 3.66 (m, 18H), 3.53 (m, 2H), 3.36 (s, 3H). LC-MS: 653.3 (M+H⁺).

### Example 9

### De Novo Synthesis of PEG-Oxybutynin

PEG-Oxybutynin was prepared. Schematically, the approach followed for this example is shown below (compound numbers in bold in the schematic correspond to the compound numbers provided in the text of this Example 9 alone).

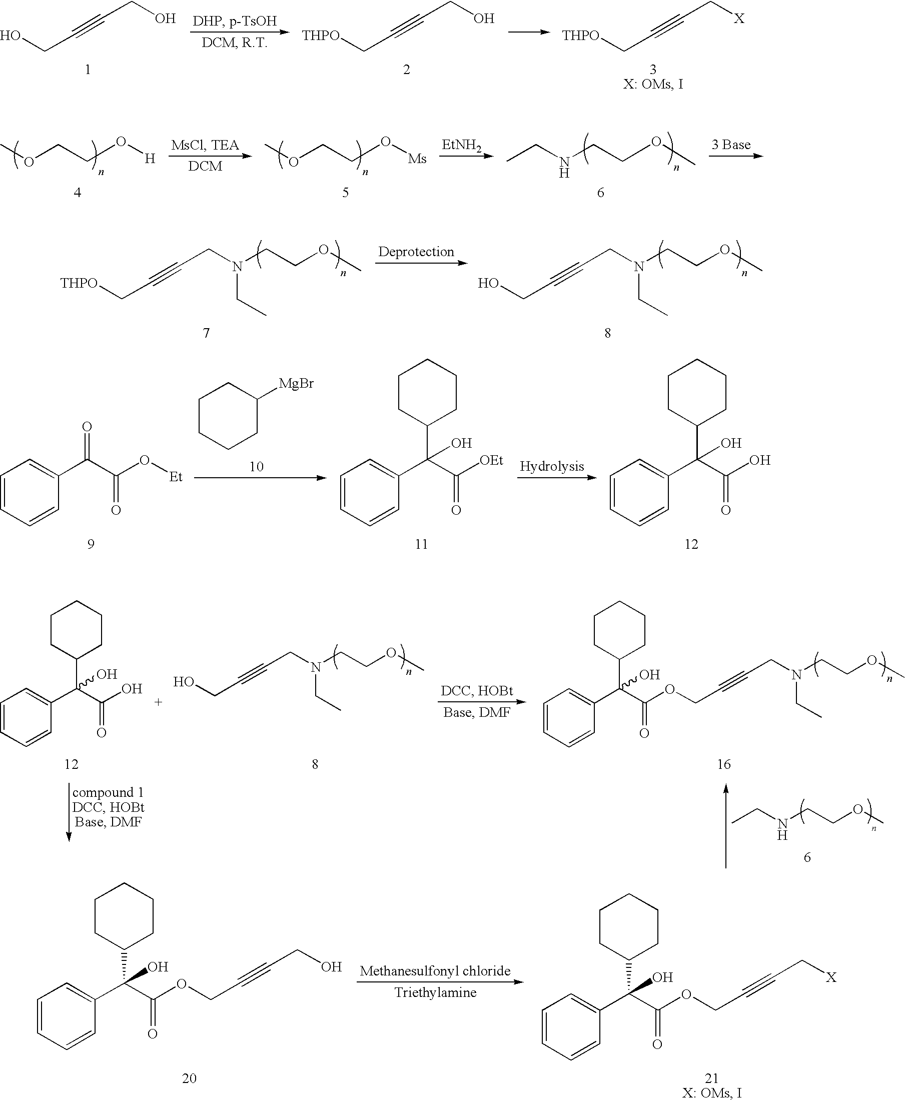

### Synthesis of 4-(tetrahydro-pyran-2-yloxy)-but-2-yn-1-ol (2)

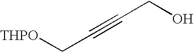

3,4-Dihydro-2H-pyron (18.3 mL, 0.196 mol) in dichloromethane (30 mL) was added dropwise over 30 minutes to a stirred solution of 2-butyne-1,4-di-ol (16.832 g, 0.194 mol) and p-TsOH (2.236 g, 11.58 mmol) in DCM (250 mL) at 0° C. After addition, the mixture was stirred at room temperature for 4 hours. Sodium bicarbonate (858 mg) was added. The mixture was stirred for another hour. Water (10 mL) was added, followed by addition of saturated aqueous potassium carbonate (150 mL). The organic phase was separated and washed with brine (200 mL), dried over anhydrous sodium sulfate, and concentrated under reduced pressure (temperature below 25° C.). The residue was separated by flash column chromatography on silica gel using 5-25% EtOAc/hexane to afford 12.88 g of product (yield: 39%), along with 12.05 g of di-protected side product. ¹H-NMR (CDCl₃): δ 4.78 (t, J=3.0-3.3 Hz, 1H), 4.37-4.21 (m, 4H), 3.86-3.78 (m, 1H), 3.55-3.50 (m, 1H), 1.83-1.64 (m, 6H).

### Synthesis of 4-(tetrahydro-pyran-2-yloxy)-but-2-ynyl mesylate (3) (x=Ms)

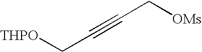

Methanesulfonyl chloride (1.0 mL, 12.80 mmol) was added dropwise to a stirred solution of 4-(tetrahydro-pyran-2-yloxy)-but-2-yn-1-ol (1.9232 g, 11.30 mmol) and TEA (2.5 mL, 17.85 mmol) in DCM (40 mL) at ° C. for five minutes. And then the resulting mixture was stirred at room temperature for 5.5 hours. Water (20 mL) was added, followed by addition of saturated aqueous NaCl solution (70 mL). The organic phase was separated and washed again with brine (60 mL), dried over Na₂SO₄, concentrated. The residue was separated by flash column chromatography on silica gel using 5-25% EtOAc/hexane to afford 1.542 g of product (yield 55%, oil), along with 400 mg of Di-4-(tetrahydro-pyran-2-yloxy)-but-2-ynyl ether (yield 22%). ¹H-NMR (CDCl₃): δ 4.88 (t, J=1.8 Hz, 2H), 4.76 (t, J=3.0-3.3 Hz, 1H), 4.37-4.24 (m, 2H), 3.80-3.76 (m, 1H), 3.54-3.50 (m, 1H), 3.11 (s, 3H), 1.78-1.51 (m, 6H).

### Synthesis of 4-(tetrahydro-pyran-2-yloxy)-but-2-ynyl iodide (3) (x=I)

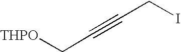

To a stirred solution of triphenylphosphine (1.4154 g, 5.34 mmol) in anhydrous dichlormethane (20 mL) at room temperature under nitrogen was added imidazole (360 mg, 5.24 mmol), followed by iodide (1.2688 g, 4.95 mmol). The mixture was stirred for three minutes, a solution of 4-(tetrahydro-pyran-2-yloxy)-but-2-yn-1-ol (708.6 mg, 4.16 mmol) in dichloromethane was added dropwise via spring. The resulting mixture was continued to be stirred for 1.5 hours. The mixture was filtered through a pad of Celite. And the solid was washed with dichloromethane. The combined organic filtration was concentrated under reduced pressure. The residue was separated with flash column chromatography on silica using 0-20% EtOAc/hexane to afford 654 mg of product in 56% yield. ¹H-NMR (CDCl₃): δ 4.77 (t, J=3.0 Hz, 1H), 4.33-4.18 (m, 2H), 3.85-3.77 (m, 1H), 3.70 (t, J=2.1 Hz, 2H), 3.56-3.49 (m, 1H), 1.85-1.62 (m, 6H).

### Synthesis of mPEG₅-OMs (5) (n=5)

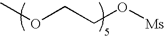

MsCl (2.5 mL, 32 mmol) was added dropwise to a stirred solution of mPEG₅-OH (5.30 g, 21 mmol) and TEA (6 mL, 42.8 mmol) in dichloromethane (50 mL) at 0° C. After addition, the resulting solution was stirred at room temperature for 22 hours. Water (10 mL) was added to quench the reaction and some saturated NaCl solution (˜40 mL) was added. The organic solution was separated and washed with brine (2×45 mL), dried over Na₂SO₄, and concentrated. The residue was dried under high vacuum to afford the product as a oil in quantitative yield. ¹H-NMR (CDCl₃): δ 4.38-4.35 (m, 2H), 3.76-3.73 (m, 2H), 3.66-3.60 (m, 14H), 3.55-3.51 (m, 2H), 3.36 (s, 3H), 3.06 (s, 3H).

Other mPEGₙ-OMs (n=3, 4, 6-20) was and/or can be synthesized following the same procedures from the corresponding mPEGₙ-OH.

### Synthesis of mPEG₄-NHEt (6) (n=4)

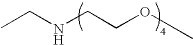

Ethylamine (70 wt % solution in water) (8 mL, 98.9 mmol) was added to a stirred solution of mPEG₄-OMs (2.75 g, 9.6 mmol) and K₂CO₃ (6.72 g, 48.16 mmol) in water (10 mL) at 0° C. Tetrabutylammonium bromide (268 mg, 0.82 mmol) was added. The resulting mixture was stirred at room temperature for 67 hours. The mixture was extracted with dichloromethane (3×20 mL). The combined organic solution was washed with brine, dried over anhydrous Na₂SO₄, concentrated to afford the product (2.373 g, 90.6% purity based on ¹H-NMR) in 95% yield. ¹H-NMR (CDCl₃): δ 3.64-3.51 (m, 14H), 3.36 (s, 3H), 2.76 (t, J=5.1-5.4 Hz, 2H), 2.63 (q, J=7.2H, 2H), 1.09 (t, J=7.2 Hz, 3H).

Other mPEGₙ-NHEt can be synthesized following the same procedures from the corresponding mPEG-OMs.

### Synthesis of ethyl-mPEG₃-4-(tetrahydropyran-2-yloxy)-but-2-ynyl]amine (7) (n=3)

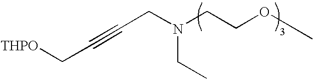

A mixture of 4-(tetrahydro-pyran-2-yloxy)-but-2-ynyl iodide (153 mg, 0.55 mmol), mPEG₃-NHEt (124 mg, 0.58 mmol, 90% pure) in THF (3 mL) in the presence of sodium bicarbonate (131 mg, 1.56 mmol) was stirred for 24.5 hours at room temperature. Water was added to quench the reaction. The mixture was concentrated under reduced pressure to remove the organic solvent. The remaining aqueous solution was extracted with EtOAc. The organic extraction was washed with brine, dried over sodium sulfate, concentrated. The residue was purified with flash column chromatography on silica gel using 0-9% MeOH/dichloromethane to afford the product (85 mg, 45% yield). ¹H-NMR (CDCl₃): δ 4.77 (t, J=3.0 Hz, 1H), 4.30-4.16 (m, 2H), 3.83-3.75 (m, 1H), 3.63-3.45 (m, 13H), 3.33 (s, 3H), 2.66 (t, J=6.0 Hz, 2H), 2.53 (q, J=7.2 Hz, 2H), 1.82-1.47 (m, 6H), 1.01 (t, J=7.2 Hz, 3H).

### Synthesis of Ethyl-mPEG₆-4-(tetrahydropyran-2-yloxy)-but-2-ynyl]amine (7) (n=6)

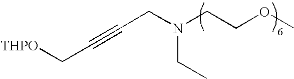

A mixture of 4-(tetrahydro-pyran-2-yloxy)-but-2-ynyl iodide (376 mg, 1.34 mmol), mPEG₆-NHEt (528 mg, 1.52 mmol, 93% pure) in THF (5 mL) in the presence of sodium bicarbonate (345 mg, 4.11 mmol) was stirred for 25 hours at room temperature. Water was added to quench the reaction. The mixture was concentrated under reduced pressure to remove the organic solvent. The remaining aqueous solution was mixed with saturated aqueous potassium carbonate solution (10 mL), extracted with EtOAc (3×40 mL). The combined organic solution was washed with brine, dried over sodium sulfate, concentrated. The residue was purified with flash column chromatography on silica gel using 0-9% MeOH/dichloromethane to afford the product (293 mg, 46% yield). ¹H-NMR (CDCl₃): δ 4.80 (t, J=3.0 Hz, 1H), 4.33-4.20 (m, 2H), 3.86-3.78 (m, 1H), 3.64-3.48 (m, 25H), 3.36 (s, 3H), 2.69 (t, J=6.0 Hz, 2H), 2.56 (q, J=7.2 Hz, 2H), 1.85-1.62 (m, 6H), 1.04 (t, J=7.2 Hz, 3H).

### Synthesis of Ethyl-mPEG₉-4-(tetrahydropyran-2-yloxy)-but-2-ynyl]amine (7) (n=9)

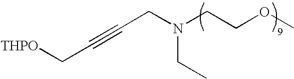

A mixture of 4-(tetrahydro-pyran-2-yloxy)-but-2-ynyl iodide (587 mg, 2.10 mmol), mPEG₉-NHEt (1.143 g, 2.38 mmol, 95% pure) in THF (5 mL) in the presence of sodium bicarbonate (642 mg, 7.64 mmol) was stirred for 26 hours at room temperature. Water was added to quench the reaction. The mixture was concentrated under reduced pressure to remove the organic solvent. The remaining aqueous solution was mixed with saturated aqueous potassium carbonate solution (10 mL), extracted with dichloromethane (3×20 mL). The combined organic solution was washed with brine, dried over sodium sulfate, concentrated. The residue was purified with flash column chromatography on silica gel using 0-5% MeOH/dichloromethane to afford the product (683 mg, 54% yield). ¹H-NMR (CDCl₃): δ 4.80 (t, J=3.0 Hz, 1H), 4.32-4.21 (m, 2H), 3.83 (m, 1H), 3.64-3.48 (m, 37H), 3.36 (s, 3H), 2.69 (t, J=6.0 Hz, 2H), 2.58 (q, J=7.2 Hz, 2H), 1.85-1.62 (m, 6H), 1.04 (t, J=7.2 Hz, 3H).

### Synthesis of 4-(mPEG₆ ethylamino)-but-2-yn-1-ol (8) (n=6)

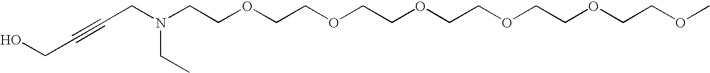

Ethyl-mPEG₆-4-(tetrahydropyran-2-yloxy)-but-2-ynyl]amine (292 mg, 0.61 mmol) was stirred in 1 N HCl ethyl ether (6 mL) at room temperature for one hour. The mixture appeared two layers. A small amount of dichloromethane was added. The resulting homogenous solution was stirred at room temperature for 17 hours. 5% aqueous sodium bicarbonate solution (20 mL) was added to quench the reaction. The mixture was extracted with dichloromethane (2×20 mL). The combined organic solution was washed with brine (2×30 mL), dried over sodium sulfate, concentrated. The residue was purified by flash column chromatography on silica gel (Biotage, 0-5% methanol/dichloromethane and 5% methanol/dichloromethane) to afford 81 mg of product in 34% yield. ¹H-NMR (500 MHz, CDCl₃): δ 4.23 (t, J=2.0 Hz, 2H), 3.62-3.56 (m, 20H), 3.52-3.50 (m, 2H), 3.47 (t, J=2.0 Hz, 2H), 3.34 (s, 3H), 2.70 (t, J=6.0 Hz, 2H and OH), 2.56 (q, J=7.0 Hz, 2H), 1.03 (t, J=7.0 Hz, 3H).

### Synthesis of Cyclohexyl-hydroxy-phenylacetic acid (11)

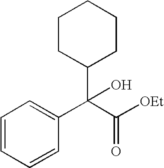

A 250 mL round-bottom flask was charged with anhydrous THF (120 mL) at room temperature and then cooled to 0° C. with water/ice bath. Cyclohexylmagnesium chloride solution (2.0 M in ethyl ether) (56 mL, 112 mmol) was added. A solution of ethyl benzoylformate (14.89 g, 79.41 mmol) in THF (20 mL) was added dropwise over 30 minutes. More of THF (10 mL) was added to wash the addition funnel. The resulting mixture was stirred at 0° C. for 15 minutes, and then at room temperature for three hours. The reaction mixture was poured into saturated aqueous ammonium chloride (150 mL). Water (15 mL) was added. The mixture was concentrated to remove the organic solvents. The remaining solution was extracted with EtOAc (2×100 mL). The extraction was washed with brine, dried over sodium sulfate, concentrated to afford a slight green residue. The residue was purified with flash column chromatography on silica gel using 0-8% EtOAc/hexane (20 CV, 40 M column, biotage) to afford 14.955 g of product in 72% yield. ¹H-NMR (400 MHz, CDCl₃).

### Synthesis of 2-Cyclohexyl-2-Phenylglycolic Acid (12)

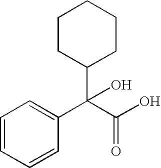

To a solution of cyclohexyl-hydroxy-phenylacetic acid (1.04 g, 3.96 mmol) in methanol (20 mL) was added a 1 N NaOH (8 mL). The reaction mixture was allowed to warm to 80° C. and stirred for 3.5 hours. After cooling to room temperature, the mixture was extracted with ethyl ether. The combined organic solution was washed with brine, dried over sodium sulfate, and concentrated in vacuo to give crude product as a white solid. The solid was recrystallized with hexane and dichloromethane to afford 900 mg of 2-cyclohexyl-2-phenylglycolic acid in 97% yield. ¹H-NMR (CDCl₃).

### Synthesis of mPEG₆-Oxybutynin (16) (n=6)

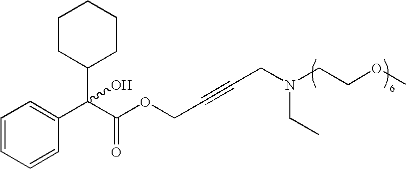

N-methyl morphinone (40 μL, 0.36 mmol) was added to a stirred solution of 2-cyclohexyl-2-phenylglycolic acid (12) (35.5 mg, 0.15 mmol) and 4-(mPEG₆ ethylamino)-but-2-yn-1-ol (8) (n=6) (45 mg, 0.12 mmol) in anhydrous DMF (2 mL) at room temperature. 1-Hydroxybenzotriazole (HOBt) (28.4 mg, 0.21 mmol) was added. The mixture was stirred at room temperature for 30 minutes, N,N′-dicyclohexylcarbodiimide (32.5 mg, 0.16 mmol) was added. The resulting mixture was stirred at room temperature for 20 hours. Water was added to quench the reaction. The mixture was extracted with EtOAc (3×15 mL). The combined organic solution was washed with brine (2×30 mL), dried over Na₂SO₄, and concentrated. The residue was purified with flash column chromatography on silica gel using 0-10% MeOH/dichloromethane to afford the product (16) (n=6) (25 mg). ¹H-NMR (500 MHz, CDCl₃): δ 7.65-7.63 (m, 2H), 7.35-7.32 (m, 2H), 7.27-7.25 (m, 1H), 4.84-4.70 (m, 2H), 3.65-3.58 (m, 18H), 3.55-3.52 (m, 4H), 3.47 (m, 2H), 3.37 (s, 3H), 2.63 (t, J=6.0 Hz, 2H), 2.49 (q, J=7.0 Hz, 2H), 2.25 (m, 1H), 2.04 (br, s, 1H), 1.79-1.77 (m, 1H), 1.64 (m, 1H), 1.54-1.52 (m, 2H), 1.46-1.38 (m, 1H), 1.35-1.26 (m, 1H), 1.20-1.06 (m, 4H), 1.02 (t, J=7.0 Hz, 3H). LC-MS: 608.3 (MH⁺).

### Synthesis of Cyclohexyl Hydroxy Phenyl Acetic Acid 4-Hydroxy But-2-ynyl Ester (20)

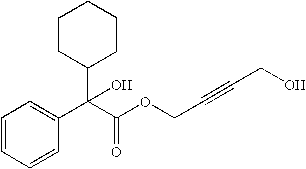

Method I:

HOBt (135.7 mg, 1.0 mmol) was added a stirred solution of 2-cyclohexyl-2-phenylglycolic acid 12 (240 mg, 1.0 mmol) and 2-butyne-1,4-diol (87 mg, 1.0 mmol) in anhydrous DMF (7.0 mL), cooled to 0° C. N-methyl morphinone (0.25 mL, 2.26 mmol) was added. The resulting mixture was stirred at 0° C. for 30 minutes. DCC (216.5 mg, 1.05 mmol) was added. The resulting mixture was stirred at 0° C. for 30 minutes, and then at room temperature for 21.5 hours. EtOAc (20 mL) was added and the white precipitate removed by filtration. The organic solution was separated and the aqueous solution was extracted with EtOAc (2×25 mL). The combined organic solution was washed with brine, dried over Na₂SO₄, concentrated. The residue was separated with flash column chromatography on silica gel using 0-20% EtOAc/hexane to afford the product (20) (50 mg, 17% yield), along with cyclohexyl phenyl acetic acid 4-(2-cyclohexyl-2-hydroxy-2-phenyl acetoxy)-but-2-ynyl ester (26) (99 mg, 37% yield) (as shown in the following structure).

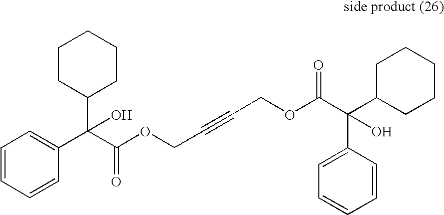

¹H-NMR (500 MHz, CDCl₃) for compound (20): δ 7.66-7.64 (m, 2H), 7.37-7.34 (m, 2H), 7.30-7.27 (m, 1H), 4.87-4.72 (m, 2H), 4.29-4.27 (m, 2H), 3.57 (s, 1H), 2.29-2.23 (m, 1H), 1.82-1.79 (m, 1H), 1.66-1.64 (m, 2H), 1.54-1.51 (m, 1H), 1.47-1.39 (m, 1H), 1.37-1.29 (m, 1H), 1.22-1.07 (m, 4H). LC-MS: 325.1 (M+Na⁺).

¹H-NMR (500 MHz, CDCl₃) for the side product—cyclohexyl phenyl acetic acid 4-(2-cyclohexyl-2-hydroxy-2-phenyl acetoxy)-but-2-ynyl este (26): δ 7.64-7.62 (m, 4H), 7.36-7.3 (m, 4H), 7.30-7.26 (m, 2H), 4.84-4.68 (m, 4H), 3.543 (s, 1H), 3.535 (s, 1H), 2.24 (m, 2H), 1.82-1.79 (m, 2H), 1.66-1.65 (m, 4H), 1.52-1.47 (m, 2H), 1.44-1.38 (m, 2H), 1.22-1.07 (m, 8H). LC-MS: 541.2 (M+Na⁺).

Method II:

A solution of 2-cyclohexyl-2-phenylglycolic acid (12) (579 mg, 2.47 mmol) and 1,1′-carbonyldiimidazole (462 mg, 2.85 mmol) was stirred at 50° C. for 5 hours, cooled to room temperature. The solution was added to a stirred solution of 2-butyne-1,4-diol (1) (1.0085 g, 11.60 mmol) and triethylamine (2.0 mL, 14.28 mmol) in anhydrous DMF (15 mL) at −70° C. (isopropanol/dry ice) over 5 minutes. The resulting mixture was stirred at −70° C. for one hour. The cooling bath was removed and the reaction mixture was allowed to warm up to room temperature and continued to stir at room temperature for 18 hours. Water was added to quench the reaction. The mixture was extracted with ethyl acetate (3×25 mL). The combined organic solution was washed with brine (2×50 mL), dried over anhydrous sodium sulfate, concentrated. The residue was separated with flash column chromatography on silica gel using 0-5% methanol in dichloromethane to afford the product (20) (509 mg) in 68% yield, along with the side product—cyclohexyl phenyl acetic acid 4-(2-cyclohexyl-2-hydroxy-2-phenyl acetoxy)-but-2-ynyl ester (26) (129 mg, 20% yield).

### Synthesis of Cyclohexyl Hydroxy Phenyl Acetic Acid 4-Methanesulfonyloxy But-2-ynyl Ester (21) (X=OMs)

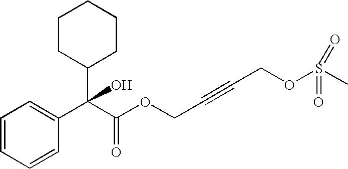

Cyclohexyl-hydroxy-phenyl-acetic acid 4-hydroxy but-2-ynyl ester (20) (277 mg, 0.92 mmol) was dissolved in dichloromethane (5 mL), cooled to 0° C. Triethylamine (0.2 mL, 1.43 mmol) was added. Methanesulfonyl chloride (75 μL, 0.96 mmol) was added dropwise with syringe. The resulting mixture was stirred at 0° C. for 40 minutes, at room temperature for 19 hours. Water was added to quench the reaction. Small of saturated sodium chloride (10 mL) was added. The organic phase was separated and the aqueous phase was extracted with dichloromethane (15 mL). The combined organic solution was washed with brine, dried over anhydrous sodium sulfate, concentrated. The residue was separated with flash column chromatography on silica gel using 5-50% ethyl acetate in hexane to afford the product (21) (98 mg) in 28% yield, along with a side product, cyclohexy-hydroxy phenyl acetic acid 4-[4-(2-cyclohexy-2-hydroxy-2-phenyl-acetoxy)-but-2-ynyl ester 27 (117 mg, 43% yield) (as shown in the following structure).

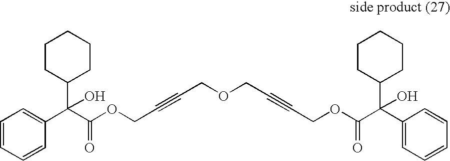

¹H-NMR (500 MHz, CDCl₃) for compound 20: δ 7.63-7.61 (m, 2H), 7.36-7.33 (m, 2H), 7.30-7.26 (m, 1H), 4.86-4.76 (m, 4H), 3.54 (s, 1H), 2.99 (s, 3H), 2.27-2.22 (m, 1H), 1.82-1.79 (m, 1H), 1.66-1.64 (m, 2H), 1.50-1.39 (m, 2H), 1.37-1.27 (m, 1H), 1.21-1.06 (m, 4H). LC-MS: 398.1 (M⁺+18), 403 (M+Na⁺).

¹H-NMR (500 MHz, CDCl₃) for the side product 27: δ 7.66-7.64 (m, 4H), 7.37-7.34 (m, 4H), 7.30-7.26 (m, 2H), 4.89-4.72 (m, 4H), 4.13 (t, J=2.0 Hz, 4H), 3.58 (s, 2H), 2.30-2.24 (m, 2H), 1.83-1.80 (m, 2H), 1.66-1.65 (m, 4H), 1.54-1.52 (m, 2H), 1.48-1.40 (m, 2H), 1.38-1.29 (m, 2H), 1.22-1.08 (m, 8H).

### Synthesis of mPEG₄-Oxybutynin (16) (n=4)

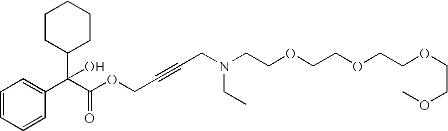

A mixture of the mesylate (21) (X═OMs) (98 mg, 0.26 mmol) and mPEG₄-NHEt (6) (n=4) (purity: 90.6%) (97 mg, 0.38 mmol) and potassium carbonate (113.8 mg, 0.82 mmol) in acetonitrile (3 mL) was stirred at room temperature for 65 hours. The reaction mixture was filtered and washed with dichloromethane. The solution was concentrated at reduced pressure. The residue was purified with flash column chromatography on silica gel using 0-10% methanol in dichloromethane to afford the product (104 mg) in 77% yield as oil. ¹H-NMR (500 MHz, CDCl₃): δ 7.65-7.64 (m, 2H), 7.35-7.32 (m, 2H), 7.28-7.25 (m, 1H), 4.84-4.70 (m, 2H), 3.66-3.59 (m, 11H), 3.56-3.53 (m, 4H), 3.46 (m, 2H), 3.38 (s, 3H), 2.62 (t, J=6.0 Hz, 2H), 2.49 (q, J=7.0 Hz, 2H), 2.28-2.23 (m, 1H), 1.80-1.77 (m, 1H), 1.64 (m, 1H), 1.55-1.52 (m, 2H), 1.47-1.38 (m, 1H), 1.35-1.26 (m, 1H), 1.20-1.06 (m, 4H), 1.02 (t, J=7.0 Hz, 3H). LC-MS: 520.2 (MH⁺).

Structure (16) having a variety of oligomer sizes can be prepared using the same approach but substituting an oligomer having a different size.

### Example 10

### De Novo Synthesis of PEG-Atazanavir

PEG-atazanavir was prepared. Schematically, the approach followed for this example is shown below (compound numbers in bold in the schematic correspond to the compound numbers provided in the text of this Example 10 alone).

### Schematic for Synthesizing the Reagent

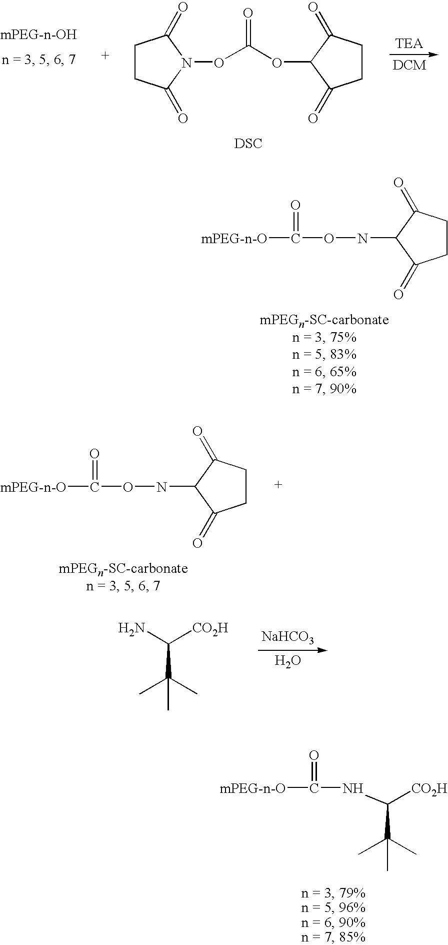

mPEG₃-SC-Carbonate

Into a 100 mL flask was placed mPEG₃-OH (2.0 g, 12.1 mmol) and anhydrous dichloromethane (25 mL). The clear solution was cooled to 0° C., and then triethylamine (1.86 mL, 13.4 mmol, 1.1 equivalents) was added slowly. The solution was stirred for 15 minutes at 0° C., and then was added to a second flask containing a suspension of DSC (3.1 g, 12.1 mmol) in dichloromethane (20 mL). The reaction mixture was allowed to equilibrate to room temperature. After approximately 18 hours, the light-yellow reaction mixture was diluted with dichloromethane (60 mL), transferred to a separatory funnel, and partitioned with deionized water (100 mL). The aqueous layer was extracted with dichloromethane (4×80 mL). The combined organics were washed with water, saturated sodium bicarbonate, and saturated sodium chloride. The dried organic layer was filtered, concentrated under reduced pressure and dried overnight under high vacuum, to give 2.79 g (75%) of mPEG₃-SC-carbonate as a light yellow oil. ¹H NMR (CDCl₃) δ 4.40 (m, 2H), 3.80 (m, 2H), 3.70 (bs, 6H), 3.60 (m, 2H), 3.35 (s, 3H), 2.80 (s, 4H); LC/MS=306 (M+1).

mPEG₅-SC-Carbonate

Into a 100 mL flask was placed mPEG₅-OH (2.0 g, 7.92 mmol) and anhydrous dichloromethane (15 mL). The clear solution was cooled to 0° C., and then triethylamine (1.32 mL, 9.51 mmol, 1.2 equivalents) was added slowly. The solution was stirred for 15 minutes at 0° C., and then was added to a second flask containing a suspension of DSC (2.02 g, 7.92 mmol) in dichloromethane (15 mL). The reaction mixture was allowed to equilibrate to room temperature. After approximately 18 hours, the light-yellow reaction mixture was diluted with dichloromethane (40 mL), transferred to a separatory funnel, and partitioned with deionized water (80 mL). The aqueous layer was extracted with dichloromethane (4×50 mL). The combined organics were washed with water, saturated sodium bicarbonate, and saturated sodium chloride. The dried organic layer was filtered, concentrated under reduced pressure and dried overnight under high vacuum, to give 2.59 g (83%) of mPEG₅-SC-carbonate as a light yellow oil. ¹H NMR (CDCl₃) δ 4.45 (m, 2H), 3.75 (m, 2H), 3.68 (bs, 16H), 3.55 (m, 2H), 3.34 (s, 3H), 2.80 (s, 4H); LC/MS=394 (M+1).

mPEG₆-SC-Carbonate

Into a 100 mL flask was placed mPEG₆-OH (2.0 g, 6.74 mmol) and anhydrous dichloromethane (12 mL). The clear solution was cooled to 0° C., and then triethylamine (1.12 mL, 8.10 mmol, 1.2 equivalents) was added slowly. The solution was stirred for 15 minutes at 0° C., and then was added to a second flask containing a suspension of DSC (1.73 g, 6.74 mmol) in dichloromethane (15 mL). The reaction mixture was allowed to equilibrate to room temperature. After approximately 18 hours, the light-yellow reaction mixture was diluted with dichloromethane (50 mL), transferred to a separatory funnel, and partitioned with deionized water (80 mL). The aqueous layer was extracted with dichloromethane (4×50 mL). The combined organics were washed with water, saturated sodium bicarbonate, and saturated sodium chloride. The dried organic layer was filtered, concentrated under reduced pressure and dried overnight under high vacuum, to give 1.92 g (65%) of mPEG₆-SC-carbonate as a light yellow oil. NMR (CDCl₃) δ 4.48 (m, 2H), 3.78 (m, 2H), 3.68 (bs, 20H), 3.58 (m, 2H), 3.38 (s, 3H), 2.84 (s, 4H); LC/MS=438 (M+1).

mPEG₇-SC-Carbonate

Into a 100 mL flask was placed mPEG₇-OH (2.0 g, 5.87 mmol) and anhydrous dichloromethane (15 mL). The clear solution was cooled to 0° C., and then triethylamine (1.22 mL, 8.81 mmol, 1.5 equivalents) was added slowly. The solution was stirred for 15 minutes at 0° C., and then was added to a second flask containing a suspension of DSC (2.25 g, 8.81 mmol) in dichloromethane (15 mL). The reaction mixture was allowed to equilibrate to room temperature. After approximately 18 hours, the light-yellow reaction mixture was diluted with dichloromethane (50 mL), transferred to a separatory funnel, and partitioned with deionized water (80 mL). The aqueous layer was extracted with dichloromethane (4×50 mL). The combined organics were washed with water, saturated sodium bicarbonate, and saturated sodium chloride. The dried organic layer was filtered, concentrated under reduced pressure and dried overnight under high vacuum, to give 2.82 g (90%) of mPEG₇-SC-carbonate as a light yellow oil. ¹H NMR (CDCl₃) δ 4.45 (m, 2H), 3.78 (m, 2H), 3.65 (bs, 24H), 3.58 (m, 2H), 3.39 (s, 3H), 2.85 (s, 4H); LC/MS=482 (M+1).

mPEG₃-L-tert-Leucine

Into a 125 mL flask was placed L-tert-Leucine (0.43 g, 3.27 mmol) and deionized water (12 mL). The solution was stirred for 30 minutes until clear, followed by the addition of solid sodium bicarbonate (1.27 g, 15.0 mmol, 4.6 equivalents). The cloudy solution was stirred at room temperature, under nitrogen. In a second flask the mPEG₃-SC-carbonate (1.24 g, 4.09 mmol, 1.25 equiv.) was taken up in deionized water (12 mL) and this solution was added all at once to the basic L-tert-Leucine solution. The cloudy light-yellow reaction mixture was stirred at room temperature, under nitrogen. After approximately 20 hours, the clear mixture was cooled to 0° C., and carefully acidified with 2 N HCl to pH 1 (20 mL). The acidic mixture was transferred to a separatory funnel and partitioned with dichloromethane (50 mL) and additional water (50 mL). The aqueous layer was extracted with dichloromethane (4×50 mL). The combined organic layers were washed with water and saturated sodium chloride, and dried over sodium sulfate. The dried organic layer was filtered, concentrated under reduced pressure and dried under high vacuum overnight, to give 0.83 g (79%) of mPEG₃-L-tert-Leucine as a pale yellow oil. ¹H NMR (CDCl₃) δ 5.45 (d, 1H), 4.26-4.35 (m, 2H), 4.14 (m, 1H), 3.70 (bs, 17H), 3.65 (m, 2H), 3.32 (s, 3H), 0.96 (s, 9H); LC/MS=322 (M+1).

mPEG₅-L-tert-Leucine

Into a 250 mL flask was placed L-tert-Leucine (0.68 g , 5.21 mmol) and deionized water (20 mL). The solution was stirred for 30 minutes until clear, followed by the addition of solid sodium bicarbonate (1.96 g, 23.3 mmol, 4.5 equivalents). The cloudy solution was stirred at room temperature, under nitrogen. In a second flask the mPEG₅-SC-carbonate (3) was taken up in deionized water (20 mL) and this solution was added all at once to the basic L-tert-Leucine solution. The cloudy light-yellow reaction mixture was stirred at room temperature, under nitrogen. After approximately 18 hours, the clear mixture was cooled to 0° C., and carefully acidified with 2 N HCl to pH 1 (18 mL). The acidic mixture was transferred to a separatory funnel and partitioned with dichloromethane (50 mL) and additional water (50 mL). The aqueous layer was extracted with dichloromethane (4×50 mL). The combined organic layers were washed with water and saturated sodium chloride, and dried over sodium sulfate. The dried organic layer was filtered, concentrated under reduced pressure and dried under high vacuum overnight, to give 2.04 g (96%) of mPEG₅-L-tert-Leucine as a pale yellow oil. ¹H NMR (CDCl₃) δ 5.45 (d, 1H), 4.26-4.35 (m, 2H), 4.14 (m, 1H), 3.70 (bs 17H), 3.65 (m, 2H), 3.38 (s, 3H), 1.02 (s, 9H); LC/MS=410 (M+1).

mPEG₆-L-tert-Leucine

Into a 250 mL flask was placed L-tert-Leucine (0.45 g , 3.47 mmol) and deionized water (15 mL). The solution was stirred for 30 minutes until clear, followed by the addition of solid sodium bicarbonate (1.31 g, 15.6 mmol, 4.5 equivalents). The cloudy solution was stirred at room temperature, under nitrogen. In a second flask the mPEG₆-SC-carbonate (1.9 gm, 4.34 mmol, 1.25 equiv.) was taken up in deionized water (15 mL) and this solution was added all at once to the basic L-tert-Leucine solution. The cloudy light-yellow reaction mixture was stirred at room temperature, under nitrogen. After approximately 18 hours, the clear mixture was cooled to 0° C., and carefully acidified with 2 N HCl to pH 1 (10 mL). The acidic mixture was transferred to a separatory funnel and partitioned with dichloromethane (50 mL) and additional water (50 mL). The aqueous layer was extracted with dichloromethane (4×50 mL). The combined organic layers were washed with water and saturated sodium chloride, and dried over sodium sulfate. The dried organic layer was filtered, concentrated under reduced pressure and dried under high vacuum overnight, to give 1.39 g (90%) of mPEG₆-L-tert-Leucine as a pale yellow oil. ¹H NMR (CDCl₃) δ 5.47 (d, 1H), 4.10-4.30 (m, 2H), 4.14 (m, 1H), 3.70 (bs, 20H), 3.65 (m, 2H), 3.38 (s, 3H), 1.02 (s, 9H); LC/MS=454 (M+1).

mPEG₇-L-tert-Leucine

Into a 250 mL flask was placed L-tert-Leucine (0.31 g, 2.32 mmol) and deionized water (15 mL). The solution was stirred for 30 min until clear, followed by the addition of solid sodium bicarbonate (0.89 g, 10.6 mmol, 4.5 equivalents). The cloudy solution was stirred at room temperature, under nitrogen. In a second flask the mPEG₇-SC-carbonate (1.4 gm, 2.91 mmol, 1.25 equiv.) was taken up in deionized water (15 mL) and this solution was added all at once to the basic L-tert-Leucine solution. The cloudy light-yellow reaction mixture was stirred at room temperature, under nitrogen. After approximately 18 hours, the clear mixture was cooled to 0° C., and carefully acidified with 2 N HCl to pH 1 (8 mL). The acidic mixture was transferred to a separatory funnel and partitioned with dichloromethane (50 mL) and additional water (50 mL). The aqueous layer was extracted with dichloromethane (4×50 mL). The combined organic layers were washed with water and saturated sodium chloride, and dried over sodium sulfate. The dried organic layer was filtered, concentrated under reduced pressure and dried under high vacuum overnight, to give 1.0 g (85%) of mPEG₇-L-tert-Leucine as a pale yellow oil. ¹H NMR (CDCl₃) δ 5.46 (d, 1H), 4.10-4.25 (m, 2H), 4.14 (m, 1H), 3.70 (bs, 24H), 3.65 (m, 2H), 3.38 (s, 3H), 1.02 (s, 9H); LC/MS=498 (M+1).

### Schematic for Synthesizing PEG-Atazanavir

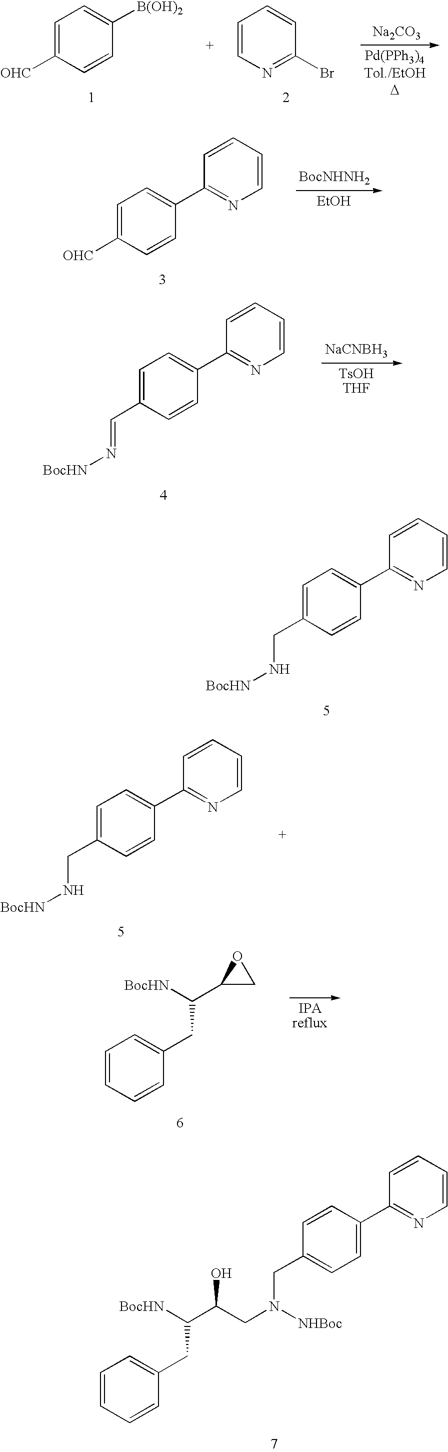

Methods

All reactions with air- or moisture-sensitive reactants and solvents were carried out under nitrogen atmosphere. In general, reagents and solvents (except PEG-based reagents) were used as purchased without further purification. Analytical thin-layer chromatography was performed on silica F₂₅₄ glass plates (Biotage). Components were visualized by UV light of 254 nm or by spraying with phosphomolybdic acid. Flash chromatography was performed on Biotage SP4 system. ¹H NMR spectra: Bruker 300 MHz; chemical shifts of signals are expressed in parts per million (ppm) and are referenced to the deuterated solvents used. MS spectra: rapid resolution Zorbax C18 column; 4.6×50 mm; 1.8 μm. HPLC method had the following parameters: column, Betasil C18, 5-μm (100×2.1 mm); flow, 0.5 mL/minute; gradient, 0-23 minutes, 20% acetonitrile/0.1% TFA in water/0.1% TFA to 100% acetonitrile/0.1% TFA; detection, 230 nm. t_{R} refers to the retention time. Abbreviations: TPTU, O-(1,2-Dihydro-2-oxo-1-pyridyl)-N,N,N′, N′-tetramethyluroniumtetrafluoroborate; DIPEA, N,N′-Diisopropylethylamine.

### 4-Pyridin-2-yl-benzaldehyde (3)

A mixture of 4-formyl-phenylboronic acid (5.0 g, 33.0 mmol) and 2-bromopyridine (5.53 g, 35.0 mmol, 1.05 equiv.) in 265 mL of 4:3 toluene/95% ethanol was degassed with nitrogen for 30 minutes and then heated under a nitrogen atmosphere, resulting in a clear solution. A slurry of Pd(PPh₃)₄ (0.77 g) in 50 mL of a 4:4 mixture of toluene and 95% ethanol was added, followed by 50 mL of 3M aqueous Na₂CO₃. The resulting mixture was gently refluxed at 77° C. After 16 hours, the reaction mixture was cooled to room temperature, and the solid removed by filtration. The filtrate was transferred to a separatory funnel, and the layers separated. The aqueous layer was extracted with toluene (3×50 mL). The combined organics were washed with water, then saturated sodium chloride, and dried over sodium sulfate. The solution was filtered, and the filtrate concentrated under reduced pressure to give a yellow oil. Purification by Biotage chromatography (40+M cartridge; gradient, 0 to 5% methanol/dichloromethane) gave 4.13 g (68%) of (3) as a light-yellow solid. TLC R_{f} (hexane/ethyl acetate, 2:1)=0.25; ¹H NMR (CDCl₃) δ 10.1 (s, HCO), 8.77 (d, 1H), 8.20 (d, 2H), 8.00 (d, 2H), 7.81 (m, 2H), 7.31 (q, 1H); MS (M)⁺=184; HPLC t_{R} 1.2 minutes.

### N-1-(tert-Butyloxycarbonyl)-N-2-[4-(pyridine-2-yl)benzylidene]-hydrazone (4)

To a 100 mL flask was added (3) (0.50 g, 2.73 mmol), tert-butyl carbazate (0.36 g, 2.73 mmol), 2-propanol (3.0 mL) and toluene (3.0 mL). The mixture was heated to reflux (85° C.) under inert atmosphere for two hours, cooled to room temperature gradually and stirred overnight under nitrogen. After 16 hours the reaction mixture was filtered, and the filter cake was washed with a cold mixture of toluene and hexane (1:3; 100 mL). The cake was dried under vacuum to afford 0.73 g (90%) of (4) as an off-white solid. TLC R_{f} (hexane/ethyl acetate, 1:2)=0.38; ¹H NMR (CDCl₃) δ 8.70 (d, 1H), 8.02 (m, 3H), 7.87 (s, 1H), 7.81 (s, 1H), 7.76 (m, 3H), 7.25 (m, 1H), 1.55 (s, 9H); MS (M)⁺=298; HPLC t_{R} 2.1 minutes.

### N′-(4-Pyridin-2-yl-benzyl)-hydrazinecarboxylic acid tert-butyl ester (5)

Into a 100 mL flask was placed (4) (0.45 g, 1.50 mmol) in THF (3.0 mL). To this solution was added 99% sodium cyanoborohydride (0.12 g, 1.80 mmol, 1.2 equivalents), followed by a solution of p-TsOH (0.35 g, 1.80 mmol, 1.2 equivalents) in THF (3.0 mL). After 1.5 hours, additional p-TsOH (0.35 g, 1.80 mmol, 1.2 equivalents) in THF (3.0 mL) was added. After 16 hours at room temperature, the THF was removed under reduced pressure. The white residue was partitioned between ethyl acetate (35 mL) and water (35 mL). The aqueous layer was extracted with ethyl acetate (3×35 mL). The combined organics were washed with water, then saturated sodium chloride, and then dried over sodium sulfate. After filtration, concentration under reduced pressure, and drying under high vacuum for 6 h, 0.41 g (91%) of (5) was obtained as a white solid. TLC R_{f} (hexane/ethyl acetate, 1:2)=0.30; ¹H NMR (DMSO-d₆) δ 8.64 (d, 1H), 8.26 (sb, HN), 8.02 (d, 2H), 7.93 (d, 1H), 7.85 (dd, 1H), 7.42 (d, 2H), 7.32 (dd, 1H), 4.80 (m, HN), 3.92 (d, 2H), 1.38 (s, 9H); MS (M)⁺=300; HPLC t_{R} 7.0 minutes.

### N′-(3-tert-Butoxycarbonylamino-2-hydroxy-4-phenyl-butyl)-N′-(4-pyridin-2-yl-benzyl)-hydrazinecarboxylic acid tert-butyl ester (7)

Into a 100 mL flask was placed (5) (1.0 g, 3.34 mmol), (6) (2S,3S)-1,2-epoxy-3-(Boc-amino)-4-phenylbutane (2.78 g, 10.5 mmol, 3.16 equivalents), and 2-propanol (15 mL). The reaction was heated to reflux. After approximately 61 hours of refluxing, the heat was removed, and the mixture cooled to room temperature. To the cooled mixture was added water/ice (50 mL). To the aqueous mixture was added dichloromethane (50 mL) and then transferred to a separatory funnel. The aqueous layer was extracted with dichloromethane (3×50 mL). The combined organics were washed with water, then saturated sodium chloride, and then dried over sodium sulfate. The dried organic solution was filtered, and the filtrate was concentrated under reduced pressure, and then dried under high vacuum overnight. The yellow foam was purified by Biotage chromatography (40+ M cartridge; 0 to 5% methanol/dichloromethane over 25 CV) to give 1.24 g (66%) of (7) as a white solid. TLC R_{f} (hexane/ethyl acetate, 1:2)=0.45; ¹H NMR (CD₃OD) δ 8.60 (d, 1H), 7.88 (m 4H), 7.50 (d, 2H), 7.36 (m, 1H), 7.25 (m, 4H), 7.18 (m, 1H), 3.93 (m, 2H), 3.70 (m, 2H), 3.0-2.6 (m, 4H), 1.33 (s, 9H), 1.30 (s, 9H); MS (M)⁺=563; HPLC t_{R} 9.6 minutes.

### 3-Amino-4-phenyl-1-[N-(4-pyridin-2-yl-benzyl)-hydrazino]-butan-2-ol trihydrochloride (8)

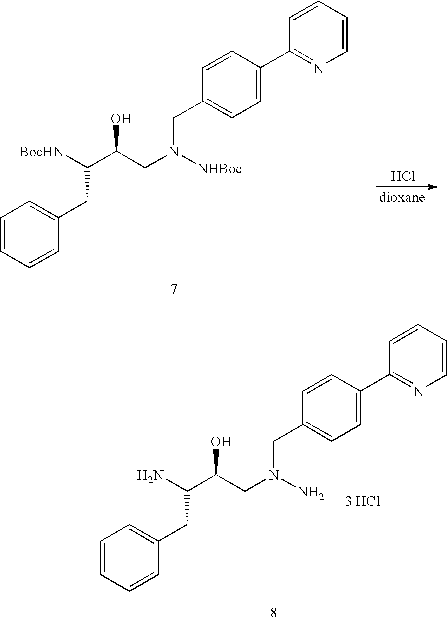

The Boc-aza-isostere (7) (1.2 g, 2.1 mmol) was taken up in 1,4-dioxane (16 mL), and stirred at room temperature, under nitrogen. After five minutes, 4N HCl (12 mL) was added via syringe. There was immediate precipitate formation, and the mixture was stirred at room temperature, under nitrogen. After approximately 18 hours, the dioxane was removed under reduced pressure. The yellow residue was azeotroped with toluene (3×25 mL), and then dried under high vacuum. After 6 hours under high vacuum, 0.92 g (91%) of (8) was obtained as a yellow solid. ¹H NMR (CD₃OD) δ 8.87 (d, 1H), 8.69 (m, 1H), 8.42 (d, 1H), 8.06 (m, 3H), 7.80 (d, 2H), 7.28 (m, 6H), 4.25 (m, 3H), 3.13 (m, 2H), 2.88 (d, 2H); MS (M)⁺=472.

### Synthesis of di-mPEGₙ-Atazanavir

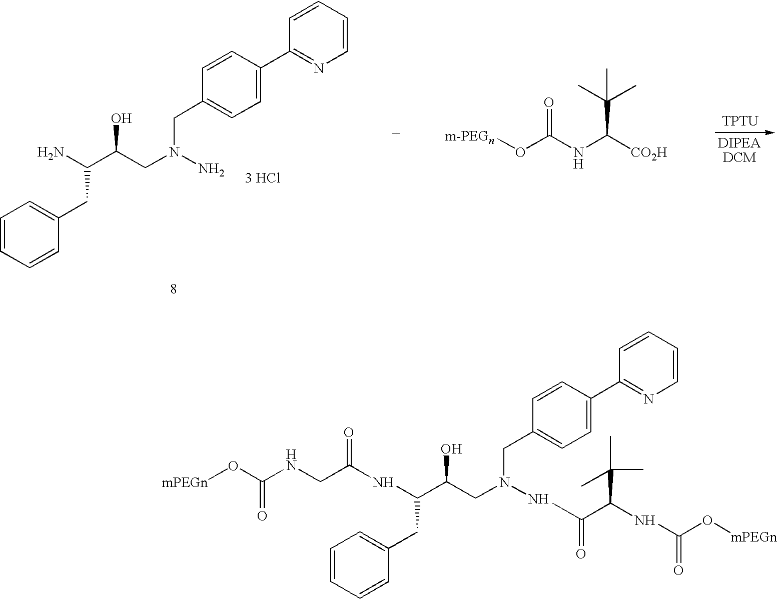

### Synthesis of di-mPEG₃-Atazanavir

Into a 100 mL flask was placed mPEG₃-tert-Leucine (0.34 gm, 1.05 mmol, 3.0 equivalents) in anhydrous dichloromethane (3 mL) and cooled to 0° C. Next, TPTU (0.31 gm, 1.05 mmol, 3.0 equiv.), and Hunigs base (0.36 mL, 2.11 mmol, 6.0 equiv.) were added. The cloudy solution was stirred at 0° C. for 15 minutes, and then the diamino backbone trihydrochloride (8) (0.16 gm, 0.35 mmol) was added, as a solid, followed by a dichloromethane rinse (3 mL). The ice bath was removed and the reaction mixture allowed to equilibrate to room temperature. After approximately 20 hours, the reaction mixture was diluted with dichloromethane (20 mL). The mixture was transferred to a separatory funnel, and partitioned with deionized water (50 mL). The aqueous layer was extracted with dichloromethane (4×30 mL). The combined organics were washed with water, saturated sodium bicarbonate, and saturated sodium chloride. The organic layer was dried over sodium sulfate. The drying agent was filtered off, and the filtrate concentrated under reduced pressure to give a yellow oil. Purification was performed using Biotage (40+M cartridge; gradient elution: 0 to 5% methanol/dichloromethane) to give 0.14 gm (45%) of di-mPEG₃-Atazanavir as a clear oil. TLC R_{f} (5% methanol/dichloromethane)=0.22; ¹H NMR (CDCl₃) δ 8.71 (d, 1H), 7.98 (d, 2H), 7.81 (m, 2H), 7.45 (d, 2H), 7.10-7.30 (m, 10H), 6.22 (d, 1H), 5.35 (d, 1H), 4.25 (m, 4H), 4.01 (m, 4H), 3.50-3.80 (m, 24H), 3.38 (s, 3H), 2.70-3.0 (m, 4H), 0.85 (d, 18H); MS (M)⁺=969; HPLC t_{R} 7.85 minutes. (96% purity).

### di-mPEG₅-Atazanavir

Into a 100 mL flask was placed m-PEG₅-tert-Leucine (2.0 gm, 4.88 mmol, 4.6 equiv.) in anhydrous dichloromethane (10 mL) and cooled to 0° C. Then added TPTU (1.45 gm, 4.88 mmol, 4.6 equiv.), and Hunigs base (1.85 mL, 10.6 mmol, 10.0 equiv.) The cloudy solution was stirred at 0° C. for 15 minutes, and then the diamino backbone trihydrochloride (8) (0.50 gm, 1.06 mmol) was added, as a solid, followed by a dichloromethane rinse (10 mL). The ice bath was removed and the reaction mixture allowed to equilibrate to room temperature. After approximately 20 hours, the reaction mixture was diluted with dichloromethane (40 mL). The mixture was transferred to a separatory funnel, and partitioned with deionized water (60 mL). The aqueous layer was extracted with dichloromethane (4×50 mL). The combined organics were washed with water, saturated sodium bicarbonate, and saturated sodium chloride. The organic layer was dried over sodium sulfate. The drying agent was filtered off, and the filtrate concentrated under reduced pressure to give a yellow oil. Purification was performed using Biotage (40+M cartridge; gradient elution: 0 to 5% methanol/dichloromethane) to give 0.70 gm (58%) of di-mPEG₅-Atazanavir as a light-yellow oil. TLC R_{f} (5% methanol/dichloromethane)=0.23; ¹H NMR (CDCl₃) δ 8.60 (d, 1H), 7.88 (d, 2H), 7.65 (m, 2H), 7.38 (d, 2H), 7.10-7.25 (m, 8H), 6.18 (d, 1H), 5.30 (m, 2H), 4.15 (m, 4H), 3.92 (m, 3H), 3.45-3.65 (m, 40H), 3.30 (s, 3H), 2.65-2.90 (m, 4H), 0.80 (d, 18H); MS (M)⁺=1146; HPLC t_{R} 7.72 minutes. (98% purity).

### di-mPEG₆-Atazanavir

Into a 100 mL flask was placed mPEG₆-tert-Leucine (0.81 gm, 1.78 mmol, 3.0 equiv.) in anhydrous dichloromethane (3 mL) and cooled to 0° C. Next, EDC (0.34 gm, 1.78 mmol, 3.0 equiv.) and HOBT (0.24 gm, 1.78 mmol, 3.0 equiv.) were added. The cloudy solution was stirred at 0° C. for 15 minutes, and then the diamino backbone trihydrochloride (8) was added (0.28 gm, 0.59 mmol), as a solid, followed by a dichloromethane rinse (5 mL). The ice bath was removed and the reaction mixture allowed to equilibrate to room temperature. After approximately 28 hours, the reaction mixture was diluted with dichloromethane (35 mL). The mixture was transferred to a separatory funnel, and partitioned with deionized water (60 mL). The aqueous layer was extracted with dichloromethane (4×50 mL). The combined organics were washed with water, saturated sodium bicarbonate, and saturated sodium chloride. The organic layer was dried over sodium sulfate. The drying agent was filtered off, and the filtrate concentrated under reduced pressure to give a yellow oil. Purification was performed using Biotage (40+M cartridge; gradient elution: 0 to 5% methanol/dichloromethane) to give 0.27 gm (40%) of di-mPEG₆-Atazanavir as a clear oil. TLC R_{f} (5% methanol/dichloromethane)=0.17; ¹H NMR. (CDCl₃) δ 8.75 (d, 1H), 78.02 (d, 2H), 7.85 (m, 2H), 7.50 (d, 2H), 7.10-7.25 (m, 6H), 6.22 (d, 1H), 5.40 (m, 2H), 4.20 (m, 4H), 4.15 (m, 3H), 3.52-3.70 (m, 48H), 3.38 (s, 3H), 2.75-2.92 (m, 4H), 0.85 (d, 18H); MS (M)⁺=1234; HPLC t_{R} 7.70 min. (96% purity).

di-mPEG₇-Atazanavir: Into a 100 mL flask was placed mPEG₇-tert-Leucine (2.13 gm, 4.29 mmol, 4.6 equiv.) in anhydrous dichloromethane (10 mL) and cooled to 0° C. Then added TPTU (1.28 gm, 4.29 mmol, 4.6 equiv.), and Hunigs base (1.14 mL, 6.53 mmol, 7.0 equiv.) The cloudy solution was stirred at 0° C. for 15 minutes, and then the diamino backbone trihydrochloride (0.44 gm, 0.93 mmol) was added, as a solid, followed by a dichloromethane rinse (10 mL). The ice bath was removed and the reaction mixture allowed to equilibrate to room temperature. After approximately 22 hours, the reaction mixture was diluted with dichloromethane (30 mL). The mixture was transferred to a separatory funnel, and partitioned with deionized water (50 mL). The aqueous layer was extracted with dichloromethane (4×50 mL). The combined organics were washed with water, saturated sodium bicarbonate, and saturated sodium chloride. The organic layer was dried over sodium sulfate. The drying agent was filtered off, and the filtrate concentrated under reduced pressure to give a yellow oil. Purification was performed using Biotage (40+M cartridge; gradient elution: 0 to 5% methanol/dichloromethane) to give 0.47 gm (38%) of di-mPEG₇-Atazanavir as a light-yellow oil. ¹H NMR (CDCl₃) δ 8.60 (d, 1H), 7.90 (d, 2H), 7.70 (m, 2H), 7.35 (d, 2H), 7.10-7.25 (m, 8H), 6.12 (d, 1H), 5.30 (m, 2H), 4.10 (m, 4H), 3.92 (m, 3H), 3.50-3.70 (m, 56H), 3.28 (s, 3H), 2.62-2.90 (m, 4H), 0.78 (d, 18H); MS (M)⁺=1321; HPLC t_{R} 7.69 min. (96% purity).

### Example 11

### De Novo Synthesis of PEG-Darunavir

### Approach A

PEG-darunavir was prepared using a first approach. Schematically, the approach followed for this example is shown below (compound numbers in bold in the schematic correspond to the compound numbers provided in the text of this Example 11 alone).

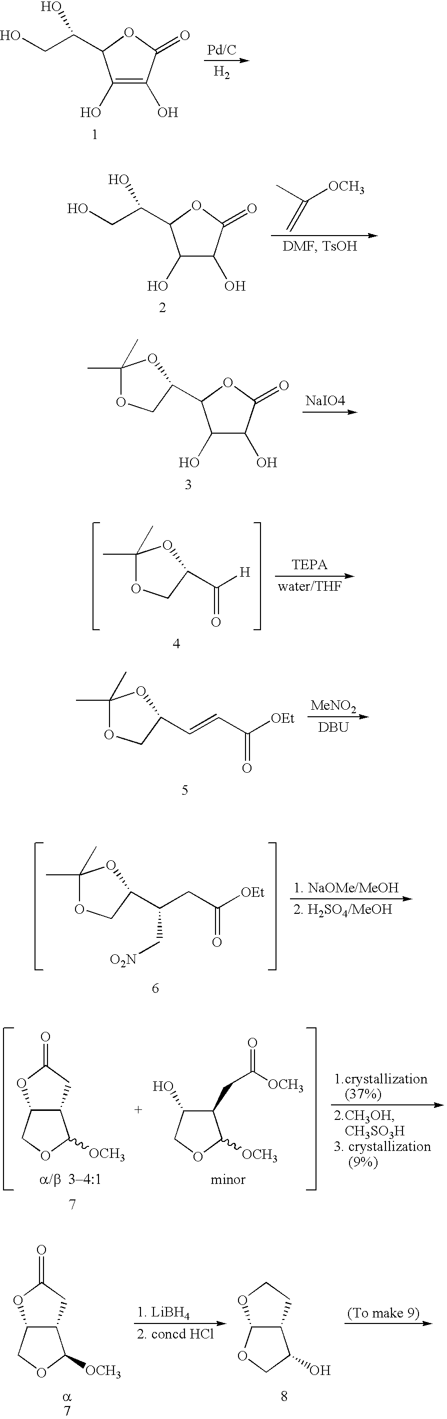

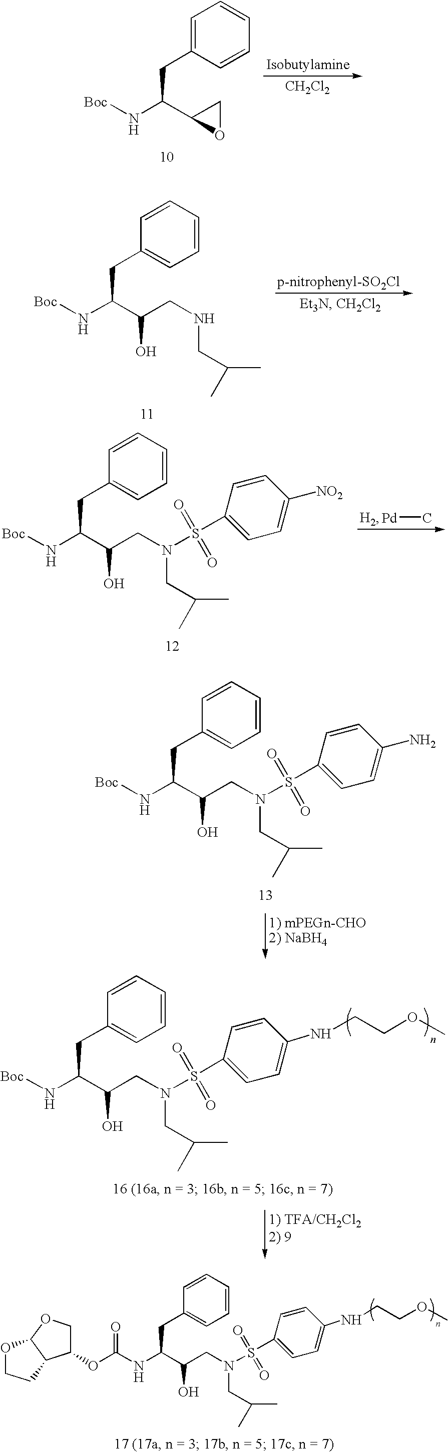

### Synthesis of L-gulono-1,4-lactone (2)

A solution of L-ascorbic acid (23.1 g, 0.13 mol) in 170 ml of water was hydrogenated using 10% Pd/C (2.2 g) in a Parr hydrogenator at 50° C. and 50 psi hydrogen pressure for 24 hours. The catalyst was removed by filtration and the water removed in vacuo to afford 23.2 g (0.13 mol, 99%) of a white crystalline solid. Upon recrystallization from methanol-ethyl acetate, 22.0 g of the desired product was obtained. ¹H NMR (DMSO): δ 5.80 (d, 1H). 5.30 (d, 1H), 4.95 (d, 1H), 4.65 (t, 1H), 4.45 (m, 1H), 4.23-4.15 (m, 2H), 3.75 (m, 1H), 3.48 (m, 2H).

### Synthesis of 5,6-isopropylidene-L-gulono-1,4-lactone (3)

A solution of L-gulono-1,4-lactone (11.08 g, 62.0 mmol) in dimethylformamide (100 ml) was cooled to 10° C. and p-toluenesulfonic acid (0.09 g, 0.50 mmol) was added portionwise with stirring. To the resultant solution, isopropenyl methyl ether (5.83 g, 80.5 mmol) was added dropwise at 10° C. The cooling bath was removed and the solution was further stirred at room temperature for 24 hours. The solution was then treated with sodium carbonate decahydrate (11 g) and the suspension was vigorously stirred for two hours. The solid was removed by filtration and mother liquor (filtrate) was concentrated using a rotary evaporator. The yellow residue was recrystallized from toluene (25 ml). The product was isolated by suction, washed with hexane/ethanol (9:1, 50 ml), and dried: yield of colorless crystalline (3): 11.22 g (82.7%). ¹H NMR (DMSO): δ 5.87 (d, 1H), 5.42 (br. 1H), 4.43 (t, 1H), 4.41-4.21 (m, 3H). 4.06 (m, 1H), 3.75 (m, 1H), 1.35 (s, 3H), 1.30, (s, 3H).

### Synthesis of E-R-3-(2,2-dimethyl-[1,3]dioxolan-4-yl)-acrylic acid ethylester (5)

To a well-stirred slurry of KIO₄ (10.60 g, 0.046 mol, 2.3 eq), KHCO₃ (4.60 g, 0.046 mol, 2.3 equiv) in water (24 g) was added dropwise a solution of L-5,6-O-isopropylidene-gulono-1,4-lactone (4.37 g, 0.020 mol) in water (2.70 g) and THF (22.90 g) during three hours at 32-34° C. The reaction mixture was stirred for 4.5 hours at 32° C. The reaction mixture was cooled to 5° C. and kept at this temperature for 14 hours. The solids were removed by filtration and the cake was washed with THF (3.0 mL) and with another portion of THF (4.0 mL) by reslurrying. To the filtrate was added dropwise under stirring triethyl phosphonoacetate (TEPA, 3.90 g, 0.017 mol) during 25 minutes at 13-17° C. Subsequently, K₂CO₃ (16.80 g) was added portionwise during 30 minutes at 17-25° C. The reaction mixture was stirred for another 17 hours at 20° C. The aqueous and THF phases were separated and the aqueous phase extracted twice with 100 mL of toluene. The combined THF and toluene phases were concentrated in vacuo giving 2.80 g of a light yellow liquid. ¹H NMR indicated the presence of E-R-3-(2,2-dimethyl-[1,3]dioxolan-4-yl)-acrylic acid ethylester (5, 78%). Thus, the crude yield of (5) was 70% yield based on (3). Of the above residue, 0.50 g was purified by flash chromatography on silica gel using 3/7 (v/v) ethyl acetate/n-heptane as the eluent. This gave 0.37 g of (5) with a purity of 96%. ¹H NMR (300 MHz, CDCl₃) δ 6.79 (1H, dd, J=16.0, 5.3 Hz), 6.01 (1H, dd, J=16.0, 0.9 Hz), 4.58 (1H, q, J=6.0 Hz), 4.16-4.06 (3H, m), 3.58 (1H, t, J=7.6 Hz), 1.35 (3H, s), 1.31 (3H, s), 1.20 (3H, t, J=7.0 Hz). ¹³C NMR (75 MHz, CDCl₃) δ 166.0 (C), 144.7 (CH), 122.4 (CH), 110.2 (C), 75.0 (CH), 68.8 (CH₂), 60.6 (CH₂), 26.5 (CH₃), 25.8 (CH₃), 14.2 (CH₃). LC-MS, calculated for C₁₀H₁₇O₄ (M+H⁺) 201.1. found 201.1.

### (3aS,4S,6aR)-4-Methoxy-tetrahydro-furo[3,4-b]furan-2(3H)-one (α-7)

To 1.75 g of non-chromatographed (5) (78 wt % pure, 1.37 g, 6.80 mmol) was added to nitromethane (458 mg, 7.50 mmol) in 5.0 mL of methanol and the solution was cooled to 10° C. Subsequently, DBU (1.03 g, 6.80 mmol) was added dropwise during 35 minutes at 10-21° C. After stirring for 18 hours at 20° C. the resulting dark-red solution was cooled to 0° C. and NaOMe (15 mL of 0.50 M solution in methanol, 7.50 mmol) was added dropwise over 30 minutes at 0° C. After 30 minutes stirring at 0° C. the reaction mixture was quenched into a solution of H₂SO₄ (2.43 g, 96%, 23.80 mmol) in methanol (2.43 g) at 0-5° C. by dropwise addition during three hours under vigorous stirring. After two hours stirring at 0-2° C. the reaction mixture was quenched into a stirred slurry of KHCO₃ (3.53 g) in water (6.80 mL) at 0-6° C. by dropwise addition during one hour. The pH was adjusted to 4.1 with H₂SO₄ (96%) at 0° C. After heating up to 20° C. the salts were removed by filtration and washed with ethyl acetate (3×3.75 mL). The wash liquor was used later on in the extractions. The mother liquor of the filtration was concentrated in vacuo to remove the methanol. To the resulting residue was added water (0.80 g) and the pH was adjusted to 4.1 with H₂SO₄ (96%). The resulting aqueous solution was extracted with ethyl acetate (7.0 mL, 4×5.0 mL). The combined organic phases were concentrated in vacuo at 35-40° C. The volatiles were coevaporated with isopropanol (3×1.40 g) giving a residue (1.46 g) consisting of a crude mixture of (α-7) and (β-7), which was dissolved in isopropanol (2.02 g) at 70° C. The insoluble material was removed and the filtrate was cooled resulting in spontaneous crystallization of (α-7). The crystals were isolated by filtration, washed with isopropanol (2×1.0 mL, 0° C.) and dried in vacuo at 40° C. until a constant weight was achieved giving (α-7) as an off-white crystalline product [390 mg, 37% yield based on (5)]. The purity was >99%. The mother liquor and wash liquors of the first (α-7) crystallization were concentrated in vacuo, methanol (1.20 mL) was added and the resulting mixture concentrated in vacuo. Methanol (1.20 mL) was added once more and the mixture concentrated in vacuo again. To the residue was added methanol (0.45 g) and methanesulfonic acid (MeSO₃H, 0.027 g, 0.28 mmol) and the solution was heated to reflux. After one hour at reflux (60-65° C.), the solution was cooled to 33° C., neutralized with triethylamine (0.029 g, 1.05 equiv based on MeSO₃H) and concentrated in vacuo. To the resulting residue was added isopropanol (1.20 mL) and the mixture was concentrated in vacuo to give 0.88 g of crude product. The residue was dissolved in isopropanol (0.37 g) at 47° C. The resulting solution was cooled down to 2° C. during 2.5 hours. The crystalline product was isolated by filtration, washed with isopropanol (3×0.20 mL, 0° C.) and dried in vacuo at 40° C. until a constant weight was achieved giving a second crop of (α-7) as an off-white crystalline product (0.098 g). The purity was >99%. Thus, the total yield of the first and second crop of (α-7) based on (5) was 46%.

The GC assay for compounds (α-7) and (β-7) was performed with an Agilent 6890 GC (EPC) and a CP-Sil 5 CB column (part number CP7680 (Varian) or equivalent) of 25 mm and with a film thickness of 5 μm using a column head pressure of 5.1 kPa, a split flow of 40 mL/minute and an injection temperature of 250° C. The used ramp was: initial temperature 50° C. (5 minutes), rate 10° C./minute, final temperature 250° C. (15 minutes). Detection was performed with an FID detector at a temperature of 250° C. The retention times were as follows: chlorobenzene (internal standard) 17.0 minutes, (α-7) 24.9 minutes, (β-7) 25.5 minutes. The retention time of (β-7) was determined by epimerizing pure (α-7) (as prepared above) to an approximately 3:1 mixture of (α-7) and (β-7) in methanol using 0.2 equiv MeSO₃H at ambient temperature during 16 hours (¹H NMR and GC-MS confirmed that only (β-7) had been formed). For the quantification of (α-7) it was assumed that the response factor of (β-7) was identical to that of (α-7). ¹H NMR (300 MHz, CDCl₃) δ 5.15 (1H, dd, J=7.4, 3.8 Hz), 4.88 (1H, s), 4.10 (1H, d, J=11.1 Hz), 3.96 (1H, dd, J=10.9, 3.8 Hz), 3.33 (3H, s), 3.10-2.99 (1H, m), 2.84 (1H, dd, J=18.2, 11.0 Hz), 2.51 (1H, dd, J=18.3, 3.7 Hz). ¹³C NMR (75 MHz, CDCl₃) δ175.9 (C), 110.0 (CH), 83.0 (CH), 70.6 (CH₂), 54.5 (CH₃), 45.1 (CH), 31.7 (CH₂). LC-MS: calculated for C₇H₁₁O₄ (M+H⁺) 159.06. found 159.06. e.e. >99% (as determined by GC).

### (3R,3aS,6aR)-Hexahydro-furo[2,3-b]furan-3-ol (8)

To a solution of (α-7) (1.42 g, 9.0 mmol) in THF (8.0 g) was added dropwise during 30 minutes a 10% solution of LiBH₄ (2.16 g, 1.1 equiv) and the reaction mixture was stirred at 50° C. for 2.5 hours. The obtained suspension was cooled to −10° C. and a 32% aqueous HCl solution (1.36 g, 0.012 mol, 1.3 equiv based on LiBH₄) was added dropwise over a period of four hours keeping the temperature <−5° C. After stirring for an additional two hours at −10° C., triethylamine (1.325 g, 0.013 mol, 1.1 equiv based on HCl) was added dropwise over one hour keeping the temperature <0° C. The reaction mixture was warmed up and concentrated at atmospheric pressure to a residual weight of approximately 5.0 g, the residue taken up in ethyl acetate (18.0 g) and concentrated once more at atmospheric pressure to a residual weight of approximately 5.0 g. The residue was taken up in ethyl acetate (18.0 g), stirred at reflux for 15 minutes and cooled to 0° C. The salts were removed by filtration and washed with cold (0° C.) ethyl acetate (2×1.5 g). The combined filtrates were concentrated in vacuo at <40° C. to a colorless oil containing 0.94 g of (8) [7.23 mmol, 80% based on (α-7), purity 87 wt % based on ¹H NMR]. The oil was purified by flash chromatography on silica gel using ethyl acetate as the eluent (R_{f}=0.56). This gave 0.89 g (6.85 mmol) of (8) with a purity of >99% which corresponds to 76% yield based on (α-7). ¹H NMR (300 MHz, DMSO-d₆) δ 5.52 (1H, d, J=4.8 Hz), 5.14 (1H, d, J=4.5 Hz), 4.27-4.17 (1H, m), 3.84-3.74 (2H, m), 3.72-3.62 (1H, m), 3.33 (1H, dd, J=22.6, 14.1 Hz), 2.77-2.66 (1H, m), 2.24-2.14 (1H, m), 1.75-1.59 (1H, m), ¹³C NMR (75 MHz, DMSO-d₆) δ 108.8 (CH), 72.1 (CH₂), 69.4 (CH), 68.8 (CH₂), 45.8 (CH), 24.6 (CH₂). ¹H NMR (300 MHz, CDCl₃) δ 5.62 (1H, d, J=4.9 Hz), 4.36 (1H, q, J=7.2 Hz), 3.94-3.77 (3H, m), 3.52 (1H, dd, J=8.9, 7.1 Hz), 3.20 (1H, s), 2.84-2.73 (1H, m), 2.30-2.20 (1H, m), 1.87-1.72 (1H, m). ¹³C NMR (75 MHz, CDCl₃) δ 109.3 (CH), 72.7 (CH₂), 70.4 (CH), 69.7 (CH₂), 46.3 (CH), 24.7 (CH₂). GC-MS: calculated for C₆H₁₁O₃ (M+H+) 131.0. found 131.0. e.e. >99% (as determined by GC). The e.e. determination of 8 was performed with an HP 5890 GC and a Supelco 24305 Betadex column of 60 mm and an internal diameter of 0.25 mm and with a film thickness of 0.25 μm using a column head pressure of 30 psi, a column flow of 1.4 mL/minute, a split flow of 37.5 mL/minute and an injection temperature of 250° C. The used ramp was: initial temperature 80° C. (1 minute), rate 4° C./minute, final temperature 180° C. (5 minutes). Detection was performed with an FID detector at a temperature of 250° C. The retention times were as follows: (8) 27.1 min, (3S,3aR,6aS)-hexahydro-furo[2,3-b]furan-3-ol [the enantiomer of (8)] 27.3 minutes. Racemic (8) required for the e.e. determination was prepared according to the same procedure as described above for optically active (8) except that racemic (α-7) was used as the starting material.

### Synthesis of Compound (9)

A solution of compound (8) (500 mg, 3.85 mmol) and N,N-disuccinimidyl (1.47 g, 5.75 mmol) in 20 mL of CH₃CN was added triethyl amine (1.10 mL, 10.40 mmol). The resulting solution was stirred at room temperature for 7 hours. The reaction mixture was concentrated under reduced pressure. The resulting residue was treated with 20 mL of saturated KHCO₃ and then extracted with ethyl acetate (150 mL×3). The organic phase was washed with water (150 mL×3) and dried over Na₂SO₄. Compound (9) (827 mg, yield 79%) was obtained after removing the solvent and dried under vacuum. ¹H NMR (300 MHz, CDCl₃) δ 2.00 (m, 1H). 2.15 (m, 1H), 2.87 (br., 4H), 3.14 (m, 1H), 3.96 (m, 2H), 4.03 (m, 1H), 4.12 (m, 1H) 5.28 (m, 1H), 5.76 (d, 1H).

### Synthesis of Compound (11)

To a stirred solution of compound (10) (962 mg, 3.65 mmol) in 2-propanol (40 mL) at room temperature was added isobutyl amine (1.60 g, 21.92 mmol). The resulting mixture was reacted at 75° C. for six hours. After this period, the reaction mixture was concentrated under reduced pressure. The resulting residue was dissolved in 5 ml of 2-propanol and concentrated again under reduced pressure. The desired product was obtained (1.17 g, yield: 95%) as a white solid; ¹H NMR (300 MHz, CDCl₃) δ 0.91 (d, 3H), 0.93 (d, 3H), 1.37 (s, 9H), 1.72 (m, 1H), 2.42 (d, 2H), 2.70 (d, 2H), 2.86 (m, 1H), 3.01 (dd, 1H), 3.48 (m, 1H), 3.84 (br., 1H), 4.74 (d, 1H), 7.20-7.33 (m, 5H); LC-MS (m/z) calcd. 336.25. found 337.25 [M+H]+.

### Synthesis of Compound (12)

To a stirred solution of the amine prepared above (1.16 g, 3.48 mmol) in a mixture of CH₂Cl₂ (30 mL) and saturated aqueous sodium bicarbonate (20 mL) at 23° C. was added 4-nitrobenzenesulfonyl chloride (1.16 g, 5.21 mmol). The resulting mixture was stirred at room temperature for 16 hours. The mixture was then extracted with CH₂Cl₂ and dried over anhydrous Na₂SO₄. Removal of solvent under reduced pressure, followed by column chromatography over silica gel (3% EtOAc in CH₂Cl₂ as the eluent), yielded compound (12) (1.29 g, 72%) as a white amorphous solid: ¹H NMR (300 MHz, CDCl₃) δ 0.86 (d, 3H), 0.87 (d, 3H), 1.36 (s, 9H), 1.84-1.92 (m, 1H), 2.86-2.95 (m, 2H), 2.98 (d, 2H), 3.19 (d, 2H), 3.75-3.82 (m, 2H), 4.64 (d, 1H), 7.22-7.32 (m, 5H), 7.95 (d, 2H), 8.32 (d, 2H); LC-MS (m/z) calcd., 521.22. found, 544.3 [M+Na]+.

### Synthesis of Compound (13)

To a solution of compound (12) (1.28 g, 2.40 mmol) in EtOAc (20 mL) was added Pd/C (100 mg). The mixture was stirred at room temperature under an H₂ (15 psi) for 10 h. The reaction mixture was filtered over Celite, and the filter cake was washed with EtOAc. Removal of solvent under reduced pressure, followed by column chromatography on silica gel (7% EtOAc in CHCl₃ as the eluent) afforded the corresponding aromatic amine (1.16 g, 98%) as a white solid: ¹H NMR (300 MHz, CDCl₃) δ 0.86 (d, 3H), 0.89 (d, 3H), 1.34 (s, 9H), 1.86 (m, 1H), 2.77 (dd, 1H), 2.89-3.15 (m, 5H), 3.85 (br., 2H), 4.05 (br., 1H), 4.17 (s, 2H), 4.65 (br., 1H), 6.71 (d, 2H), 7.19-7.30 (m, 5H), 7.58 (d, 2H); LC-MS (m/z) calcd., 491.3. found: 492.3 [M+H]⁺, 514.23. [M+Na]⁺.

### Synthesis of Compound (16)

### General Procedure for 16a, 16b and 16c

A solution of compound (13) (98 mg, 0.20 mmol) and mPEGₙ-CHO (n=3, 5 or 7, run separately) (0.30 mmol) in CH₃OH (10 mL) was stirred at 85° C. under azeotropic conditions for 90 minutes (4.0 ml of CH₃OH was removed). After this period, the reaction mixture was cooled to room temperature and sodium borohydride (20 eq.) was added in portions. The mixture was stirred at 50° C. for two hours, and then the reaction was quenched with sodium bicarbonate. 150 ml of DCM was added. The solution was washed with H₂O (3×150 ml). The organic phase was dried over sodium sulfate and was then concentrated under reduced pressure. The residue was purified by column chromatography over silica gel (2% MeOH in CHCl₃ as the eluent) to provide compound (16a), (16b) or (16c) respectively (yield, 70-80%) as colorless oil. Compound (16a) (n=3), ¹H NMR (300 MHz, CDCl₃) δ 0.86 (d, 3H), 0.89 (d, 3H), 1.33 (s, 9H), 1.82 (m, 1H), 2.77 (dd, 1H), 2.89-2.92 (m, 2H), 2.99-3.11 (m, 3H), 3.75-3.80 (m, 2H), 3.32 (m, 2H), 3.38 (s, 3H), 3.57 (m, 2H), 3.60-3.90 (m, 11H), 4.04 (br., 1H), 4.62 (d, 1H), 4.85 (t, 1H), 6.60 (d, 2H), 7.19-7.30 (m, 5H), 7.54 (d, 2H); LC-MS (m/z) calcd. 637.3. found: 638.3 [M+H]⁺. Compound (16b) (n=5), ¹H NMR (300 MHz, CDCl₃) δ 0.86 (d, 3H), 0.89 (d, 3H), 1.34 (s, 9H), 1.80-1.86 (m, 1H), 2.77 (dd, 1H), 2.89-2.92 (m, 3H), 2.99-3.11 (m, 2H), 3.32 (m, 2H), 3.36 (s, 3H), 3.54 (m, 2H), 3.58-3.90 (m, 19H), 4.65 (d, 1H), 4.98 (t, 1H), 6.59 (d, 2H), 7.19-7.30 (m, 5H), 7.54 (d, 2H). Compound (16c) (n=7), ¹H NMR (300 MHz, CDCl₃) δ 0.86 (d, 3H), 0.89 (d, 3H), 1.34 (s, 9H), 1.80-1.86 (m, 1H), 2.77 (dd, 1H), 2.89-3.11 (m, 5H), 3.32 (m, 2H), 3.36 (s, 3H), 3.54 (m, 2H), 3.58-3.90 (m, 27H), 4.65 (d, 1H), 4.98 (t, 1H), 6.59 (d, 2H), 7.19-7.30 (m, 5H), 7.54 (d, 2H).

### Synthesis of Compound (17)

### General Procedure for 17a, 17b and 17c

A solution of compound (17a), (17b) or (17c) (each run separately) (0.151 mmol) in a mixture of 30% trifluoroacetic acid in CH₂Cl₂ (4 mL) was stirred for 60 min. After this period, the reaction mixture was concentrated under reduced pressure and the resulting residue was redissolved in CH₂Cl₂ (5.0 mL). To this solution were added compound 9 (45 mg, 0.17 mmol) and triethylamine (0.155 mL, 1.51 mmol). The resulting mixture was stirred for 2 h. The reaction mixture was then concentrated under reduced pressure, and the residue was purified by column chromatography over silica gel (2% MeOH in CHCl₃ as the eluent) to provide compound (17a), (17b), and (17c), respectively (yield: 80-89%) as an oil. Compound (17a) (n=3), ¹H NMR (300 MHz, CDCl₃) δ 0.87 (d, 3H), 0.93 (d, 3H), 1.42-1.46 (m, 1H), 1.57-1.65 (m, 1H), 1.79-1.85 (m, 1H), 2.75-2.81 (m, 2H), 2.87-2.98 (m, 3H), 3.05-3.16 (m, 2H), 3.34 (m, 2H), 3.38 (s, 3H), 3.58 (m, 2H), 3.64-3.74 (m, 10H), 3.82-4.00 (m, 5H), 4.97-5.01 (m, 2H), 5.63 (d, 1H), 6.67 (d, 2H), 7.18-7.28 (m, 5H), 7.53 (d, 2H); LC-MS (m/z) calcd: 693.3. found 694.3 [M+H]⁺. Compound (17b) (n=5), ¹H NMR (300 MHz, CDCl₃) δ 0.87 (d, 3H), 0.93 (d, 3H), 1.46 (m, 1H), 1.60 (m, 1H), 1.82 (m, 1H), 2.75-2.81 (m, 2H), 2.87-2.98 (m, 3H), 3.05-3.16 (m, 2H), 3.32 (m, 2H), 3.36 (s, 3H), 3.54 (m, 2H), 3.64-3.74 (m, 18H), 3.82-3.92 (m, 5H), 4.97-5.01 (m, 2H), 5.63 (d, 1H), 6.67 (d, 2H), 7.18-7.28 (m, 5H), 7.54 (d, 2H); LC-MS (m/z) calcd. 781.4. found 782.5 [M+H]⁺. Compound (17c) (n=7), ¹H NMR (300 MHz, CDCl₃) δ 0.87 (d, 3H), 0.92 (d, 3H), 1.46 (m, 1H), 1.60 (m, 1H), 1.82 (m, 1H), 2.75-2.81 (m, 2H), 2.87-2.98 (m, 3H), 3.05-3.16 (m, 2H), 3.30 (m, 2H), 3.36 (s, 3H), 3.54 (m, 2H), 3.64-3.74 (m, 26H), 3.82-3.92 (m, 5H), 4.97-5.05 (m, 3H), 5.63 (d, 1H), 6.62 (d, 2H), 7.18-7.28 (m, 5H), 7.53 (d, 2H); LC-MS (m/z) calcd: 869.4. found 870.3 [M+H]⁺.

### Example 12

### De Novo Synthesis of PEG-Darunavir

### Approach B

PEG-darunavir was prepared using a second approach. Schematically, the approach followed for this example is shown below (compound numbers in bold in the schematic correspond to the compound numbers provided in the text of this Example 12 alone).

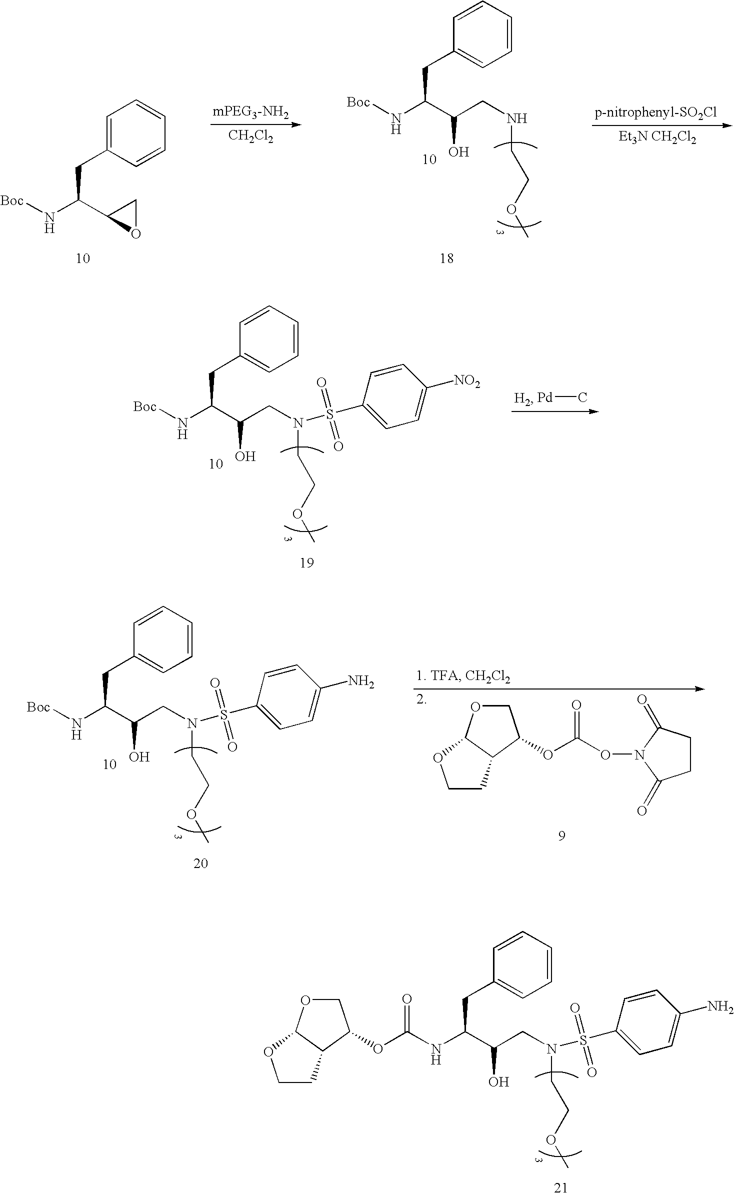

### Synthesis of Compound (18)

To a stirred solution of compound (10) (264 mg, 1.0 mmol) [prepared in accordance with the procedure for synthesizing compound (10) in Example 12] in 2-propanol (10 mL) at 23° C. was added mPEG₃-NH₂ (489 mg, 3.0 mmol). The resulting mixture was stirred at 75° C. for six hours. After this period, the reaction mixture was concentrated under reduced pressure. The residue was purified by column chromatography on silica gel (biotage, CH₃OH/DCM, 4-15% CH₃OH, 20 CV). 390 mg of corresponding amine (18) was obtained (yield, 91.5%) as sticky oil. ¹H NMR (300 MHz, CDCl₃) δ 1.35 (s, 9H), 1.85-1.89 (m, 1H), 2.70 (m, 1H), 2.86 (m, 4H), 3.00 (dd, 1H), 3.35 (s, 3H), 3.54-3.75 (m, 10H), 3.85 (m, 1H), 4.70 (d, 1H), 7.10-7.40 (m, 5H); LC-MS (m/z) calcd., 426.3. found 427.2 [M+H]⁺.

### Synthesis of Compound (19)

To a stirred solution of above prepared amine (18) (390 mg, 0.92 mmol) in a mixture of CH₂Cl₂ (15 mL) and saturated aqueous sodium bicarbonate (10 mL) at 23° C. was added 4-nitrobenzenesulfonyl chloride (304 mg, 1.38 mmol). The resulting mixture was stirred at room temperature for 16 hours. The mixture was then extracted with CH₂Cl₂ and dried over anhydrous Na₂SO₄. Removal of solvent under reduced pressure, followed by column chromatography over silica gel (biotage, DCM/CH₃OH, CH₃OH: 1-6%, 20 CV) gave the desired product (19) (455 mg, 81%) as sticky oil: ¹H NMR (300 MHz, CDCl₃) δ 1.37 (s, 9H), 2.85 (m, 1H), 3.10 (m, 2H), 3.30 (m, 1H), 3.41 (m, 2H), 3.38 (s, 3H), 3.50-3.85 (m, 11H), 3.90 (m, 1H), 4.45 (d, 1H), 4.95 (d, 1H), 7.22-7.32 (m, 5H), 7.95 (d, 2H), 8.32 (d, 2H). LC-MS (m/z) calcd., 611.3. found, 612.3 [M+H]⁺.

### Synthesis of Compound (20)

To a solution of compound (19) (455 mg, 0.74 mmol) in EtOAc (10 mL) was added Pd/C (40 mg, 10%). The mixture was stirred at room temperature under an H₂ atmosphere (30 psi) for 4.0 hours. The reaction mixture was filtered over Celite, and the filter cake was washed with EtOAc. Removal of solvent under reduced pressure afforded the corresponding aromatic amine (420 mg, 98%) as a white solid: ¹H NMR (300 MHz, CDCl₃) δ 1.36 (s, 9H), 2.90-3.10 (m, 3H), 3.10-3.30 (m, 3H), 3.37 (s, 3H), 3.56 (m, 2H), 3.63-3.90 (m, 11H), 4.54 (br., 1H), 4.88 (d, 1H), 6.65 (d, 2H), 7.19-7.30 (m, 5H), 7.53 (d, 2H); LC-MS (m/z), calcd., 581.3. found: 582.3 [M+H]⁺.

### Synthesis of Compound (21)

A solution of compound (20) (116 mg, 0.2 mmol) in a mixture of 30% trifluoroacetic acid in CH₂Cl₂ (4 mL) was stirred at room temperature for 1.0 hour. After this period, the reaction mixture was concentrated under reduced pressure and the residue was redissolved in CH₂Cl₂ (5.0 mL). To this solution were added (3R,3aS,6aR)-3 hydroxyhexahydrofuro[2,3-b]furanyl succinimidyl carbonate [compound (9)] (54 mg, 0.2 mmol) and triethylamine (0.5 mL). The resulting mixture was stirred for two hours. At which point, the solution was concentrated under reduced pressure. The resulting residue was purified by column chromatography (biotage, DCM/CH₃OH, CH₃OH: 2-6%, 20 CV) to provide compound (21) (102 mg, 80%) as a oil. ¹H NMR (300 MHz, CDCl₃) δ 1.40-1.60 (m, 1H), 1.60-1.80 (m, 1H), 1.90 (br., 1H), 2.75 (m, 1H), 2.90 (m, 1H), 3.00-3.15 (m, 2H), 3.15-3.30 (m, 3H), 3.37 (s, 3H), 3.50-3.85 (m, 12H), 3.85-3.98 (m, 4H), 4.23 (br., 2H), 4.50 (br., 1H), 5.02 (m, 1H), 5.40 (d, 1H), 5.64 (d, 1H), 6.67 (d, 2H, J) 8.6 Hz), 7.18-7.28 (m, 5H), 7.51 (d, 2H); LC-MS (m/z), calcd., 637.2. found, 638.2 [M+H]⁺.

### Example 13

### De Novo Synthesis of PEG-Darunavir

### Approach C

PEG-darunavir was prepared using a third approach. Schematically, the approach followed for this example is shown below (compound numbers in bold in the schematic correspond to the compound numbers provided in the text of this Example 13 alone).

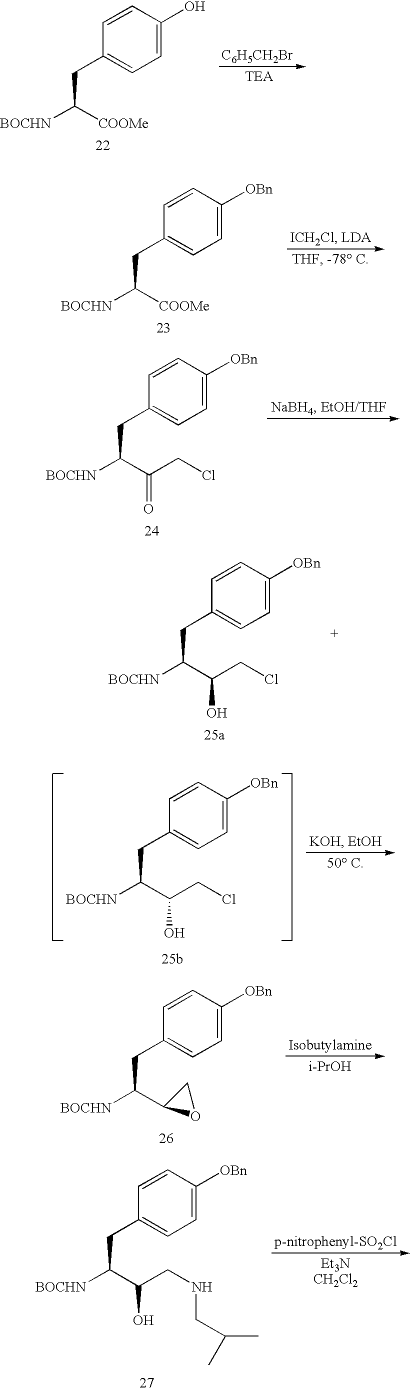

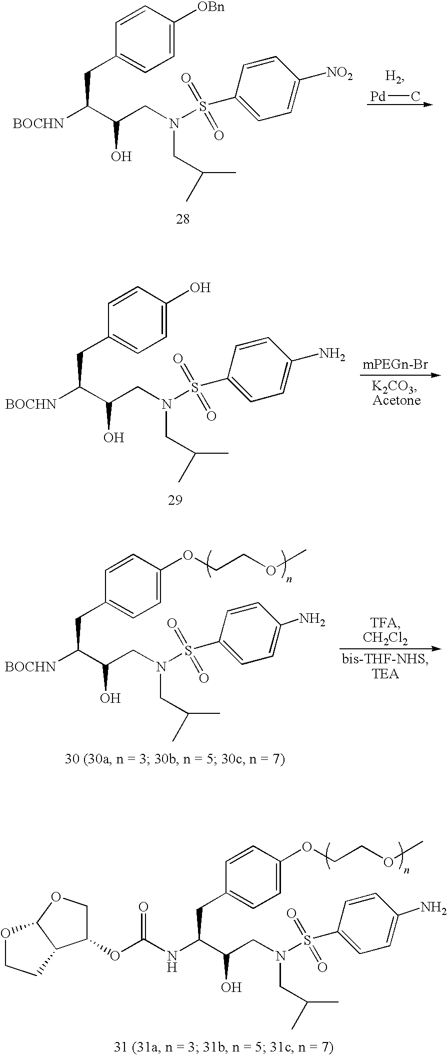

### Synthesis of Compound (23)

To a stirred solution of Boc-Tyr-OMe [compound (22), 10.33 g, 0.035 mol] and potassium carbonate (7.20 g, 0.052 mol) in acetone (45 mL) was added BnBr (6.00 g, 0.035 mol). The resulting mixture was stirred at 60° C. for 16 hours. After this period, the solid was removed by filtration and the reaction mixture was concentrated under reduced pressure. The resulting residue was purified by column chromatography (biotage: DCM/CH₃OH, CH₃OH, 0-6%, 15 CV). Product (23) was obtained (13.0 g, 96%) as a white solid; ¹H NMR (300 MHz, CDCl₃) δ 1.44 (s, 9H), 3.05 (m, 2H), 3.72 (s, 3H), 4.55 (m, 1H), 5.00 (m, 1H), 5.05 (s, 2H), 6.90 (d, 2H), 7.10 (d, 2H), 7.20-7.38 (m, 5H); LC-MS (m/z) calcd., 385.2. found 408.2 [M+Na]⁺.

### Synthesis of Compound (25)

Compound (23) (12.74 g, 0.033 mol) and iodochloromethane (23.35 g, 0.132 mol) in anhydrous THF (150 ml) was cooled to −78° C. and LDA (83 ml, 0.165 mol) was added dropwise. Upon completion of the addition, the solution was stirred at −75° C. for an additional 15 minutes. An acetic acid solution (20 ml of THF+20 ml of HOAc) was added dropwise while keeping the temperature below −70° C. Stirring was continued for 15 minutes after addition of 200 ml of toluene, then 100 ml of 1% HCl was added. The organic phase was washed with 0.5 M NaHCO₃ (10 ml) and separated. The solution was added 100 ml of ethanol and cooled to −78° C. NaBH₄ (6.3 g, 0.17 mol) was added. The mixture was stirred at −78° C. for 1 hour and then the reaction was quenched by addition of 100 ml of saturated KHSO₄. The organic phase was washed with water and dried over Na₂SO₄. The resulting solid, after solvent removal, was washed with hexane and then recrystallized from ethyl acetate. Compound (25a) [3.5 g, 30% based on compound (23)] was obtained as yellow solid. ¹H NMR (300 MHz, CDCl₃) δ 1.39 (s, 9H), 2.91 (m, 2H), 3.17 (br., 1H), 3.57 (m, 1H), 3.67 (m, 1H), 3.84 (m, 2H), 4.57 (m, 1H), 5.05 (s, 2H), 6.92 (d, 2H), 7.13 (d, 2H), 7.20-7.38 (m, 5H); LC-MS (m/z) calcd., 405.2. found 428.2 [M+Na]+.

### Synthesis of Compound (26)

Compound (25a) (2.18 g, 5.38 mmol) was suspended in a 0.1 N solution of potassium hydroxide in methanol (5.92 mmol, 59.2 ml). The resulting mixture was stirred at 50° C. for 1.5 hours. The solvent was removed under reduced pressure and the solid was dissolved in 100 ml DCM, which was subsequently washed with water (100 mL×3). The solution was dried and solvent was removed under reduced pressure. The desired product was obtained as a yellow solid (1.74 g, 88%) was obtained. ¹H NMR (300 MHz, CDCl₃) δ 1.40 (s, 9H), 2.77 (m, 3H), 2.92 (m, 2H), 3.65 (br., 1H), 4.44 (br., 1H), 5.05 (s, 2H), 6.92 (d, 2H), 7.15 (d, 2H), 7.26-7.38 (m, 5H); LC-MS (m/z) calcd., 369.2. found, 370.2 [M+H]⁺, 392.2 [M+Na]⁺.

### Synthesis of Compounds (27) & (28)

To a stirred solution of compound (26) (1.74 g, 4.80 mmol) in 2-propanol (60 mL) at 23° C. was added isobutyl amine (2.20 g, 30 mmol). The resulting mixture was reacted at 75° C. for 6 hours. After this period, the reaction mixture was concentrated under reduced pressure. The residue was dissolved in 5 ml of 2-propanol and concentrated again under reduced pressure. Compound (27) was obtained (1.97 g) as a yellow solid, which was used in next reaction without further purification.

To a stirred solution of compound (27) (1.97 g, 4.45 mmol) in a mixture of CH₂Cl₂ (40 mL) and saturated aqueous sodium bicarbonate (30 mL) at 23° C. was added 4-nitrobenzenesulfonyl chloride (1.48 g, 6.67 mmol). The resulting mixture was stirred at room temperature for 16 hours. The mixture was then extracted with CH₂Cl₂ (150 mL×2). The organic phase was washed with water (150 mL×3) and dried over anhydrous Na₂SO₄. Removal of solvent under reduced pressure, followed by column chromatography (biotage: DCM/CH₃OH, CH₃OH, 1-6%, 15CV, 6-8% 5CV), yielded compound (28) (2.14 g, 77%) as a white amorphous solid: ¹H NMR (500 MHz, CDCl₃) δ 0.87 (d, 3H), 0.89 (d, 3H), 1.37 (s, 9H), 1.87 (m, 1H), 2.86 (m, 2H), 2.99 (d, 2H), 3.19 (d, 2H), 3.72 (m, 1H), 3.79 (m, 2H), 4.61 (d, 1H), 5.05 (s, 2H), 6.90 (d, 2H), 7.14 (d, 2H), 7.35 (m, 1H), 7.44 (m, 4H), 7.95 (d, 2H), 8.34 (d, 2H); LC-MS (m/z) calcd., 627.26. found, 650.3 [M+Na]⁺.

### Synthesis of Compound (29)

To a solution of compound (28) (2.14 g, 3.41 mmol) in THF (20 mL) was added Pd/C (428 mg). The mixture was stirred at room temperature under an H₂ atmosphere (45 psi) for 48.0 hours. The reaction mixture was filtered over Celite, and the filter cake was washed with THF. Removal of the solvent under reduced pressure afforded the corresponding aromatic amine (1.48 g, 86%) as a white solid: ¹H NMR (500 MHz, CDCl₃) δ 0.86 (d, 3H), 0.90 (d, 3H), 1.36 (s, 9H), 1.85 (m, 1H), 2.77 (m, 1H), 2.84 (m, 1H), 2.90 (m, 2H), 2.92 (d, 1H), 3.07 (m, 1H), 3.71 (m, 1H), 3.77 (m, 1H), 4.16 (br., 2H), 4.72 (d, 1H), 6.66 (d, 2H), 6.75 (d, 2H), 7.09 (d, 2H), 7.52 (d, 2H).

### General Procedure for the Synthesis of Compounds (30a), (30b) and (30c)

A solution of compound (29) (152 mg, 0.30 mmol) and mPEGₙ-Br (n=3, 5 and 7, in three separate runs) (0.45 mmol) in acetone (10 mL) was stirred at 70° C. for 20 hours. After this period, the reaction mixture was cooled to room temperature and 150 mL of DCM was added. The solution was washed with water (150 mL×2). The organic phase was dried over sodium sulfate and then concentrated under reduced pressure. The resulting residue was purified by column chromatography (biotage: DCM/CH₃OH, CH₃OH, 3-6%, 15CV, 6-8% 5CV) to provide compound (30a), (30b) and (30c), respectively (yield, 70-80%) as colorless oil. Compound (30a) (n=3): ¹H NMR (500 MHz, CDCl₃) δ 0.83 (d, 3H), 0.89 (d, 3H), 1.36 (s, 9H), 1.82 (m, 1H), 2.62 (m, 1H), 2.69 (m, 1H), 2.86 (m, 1H), 2.92 (m, 3H), 3.36 (s, 3H), 3.53 (m, 2H), 3.62 (m, 2H), 3.68 (m, 2H), 3.74 (m, 4H), 3.85 (m, 3H), 4.08 (m, 2H), 4.40 (br., 2H), 4.77 (d, 1H), 6.62 (d, 2H), 6.82 (d, 2H), 7.14 (d, 2H), 7.38 (d, 2H); LC-MS (m/z) calcd., 653.3. found, 654.4 [M+H]⁺. Compound (30b) (n=5): ¹H NMR (500 MHz, CDCl₃) δ 0.83 (d, 3H), 0.89 (d, 3H), 1.37 (s, 9H), 1.81 (m, 1H), 2.60 (m, 2H), 2.85 (m, 1H), 2.92 (m, 3H), 3.35 (s, 3H), 3.52 (m, 2H), 3.61-3.65 (m, 11H), 3.70 (m, 2H), 3.75 (m, 5H), 3.85 (m, 2H), 4.07 (m, 2H), 4.49 (br., 2H), 4.73 (d, 1H), 6.62 (d, 2H), 6.82 (d, 2H), 7.15 (d, 2H), 7.34 (d, 2H); LC-MS (m/z) calcd., 741.4. found, 742.5 [M+H]⁺, 764.4. [M+Na]⁺.

Compound (30c) (n=7): ¹H NMR (500 MHz, CDCl₃) δ 0.82 (d, 3H), 0.89 (d, 3H), 1.36 (s, 9H), 1.80 (m, 1H), 2.85 (m, 1H), 2.92 (m, 3H), 3.35 (s, 3H), 3.52 (m, 2H), 3.61-3.65 (m, 19H), 3.70 (m, 2H), 3.75 (m, 5H), 3.85 (m, 2H), 4.07 (m, 2H), 4.49 (s, 2H), 4.73 (d, 1H), 6.61 (d, 2H), 6.82 (d, 2H), 7.14 (d, 2H), 7.35 (d, 2H); LC-MS (m/z) calcd., 829.4. found, 830.5 [M+H]⁺.

### General Procedure for the Synthesis of Compounds (31a), (31b) and (31c)

A solution of compound (30a), (30b) and (30c) (0.20 mmol, in three separate runs) in a mixture of 30% trifluoroacetic acid in CH₂Cl₂ (4.0 mL) was stirred at room temperature for 40 minutes. After this period, the reaction mixture was concentrated under reduced pressure and the residue was redissolved in CH₂Cl₂ (5.0 mL). To this solution were added (3R,3aS,6aR)-3 hydroxyhexahydrofuro[2,3-b]furanyl succinimidyl carbonate (54 mg, 0.20 mmol) and triethylamine (0.155 mL, 1.51 mmol). The resulting mixture was stirred for one hour. The reaction mixture was then concentrated under reduced pressure, and the residue was purified by column chromatography (biotage, DCM/CH₃OH, CH₃OH: 0-4%, 20 CV, 4-6%, 10 CV) to provide compounds (31a), (31b), and (31c), respectively (yield: 75-80%) as colorless oil. Compound (31a) (n=3): ¹H NMR (500 MHz, CDCl₃) δ 0.83 (d, 3H), 0.88 (d, 3H), 1.58 (m, 1H), 1.64 (m, 1H), 1.77 (m, 1H), 2.65 (m, 1H), 2.72 (m, 2H), 2.90 (m, 2H), 2.98 (m, 2H), 3.35 (s, 3H), 3.52 (m, 2H), 3.60 (m, 2H), 3.64 (m, 3H), 3.69 (m, 4H), 3.81 (m, 5H), 3.92 (m, 1H), 4.03 (m, 2H), 4.46 (s, 2H), 5.00 (m, 1H), 5.16 (d, 1H), 5.62 (d, 1H), 6.60 (d, 2H), 6.78 (d, 2H), 7.08 (d, 2H), 7.38 (d, 2H); LC-MS (m/z) calcd: 709.3. found 710.3 [M+H]⁺. Compound (31b) (n=5): ¹H NMR (500 MHz, CDCl₃) δ 0.86 (d, 3H), 0.92 (d, 3H), 1.71-1.85 (m, 3H), 2.65 (m, 2H), 2.78 (m, 1H), 2.97 (m, 4H), 3.36 (s, 3H), 3.54 (m, 2H), 3.64 (m, 10H), 3.68 (m, 3H), 3.75 (m, 4H), 3.69 (m, 4H), 3.85 (m, 4H), 3.90 (m, 1H), 4.00 (m, 1H), 4.10 (m, 2H), 4.50 (br., 2H), 5.06 (m, 1H), 5.12 (d, 1H), 5.66 (d, 1H), 6.64 (d, 2H), 6.82 (d, 2H), 7.13 (d, 2H), 7.37 (d, 2H); LC-MS (m/z) calcd: 797.4. found 798.4 [M+H]⁺. Compound (31c) (n=7), ¹H NMR (500 MHz, CDCl₃) δ 0.86 (d, 3H), 0.92 (d, 3H), 1.71-1.85 (m, 3H), 2.62 (m, 2H), 2.78 (m, 1H), 2.97 (m, 4H), 3.37 (s, 3H), 3.54 (m, 2H), 3.64 (m, 19H), 3.68 (m, 3H), 3.73 (m, 4H), 3.85 (m, 4H), 3.90 (m, 1H), 4.00 (m, 1H), 4.08 (m, 2H), 4.52 (br., 2H), 5.06 (m, 1H), 5.12 (d, 1H), 5.66 (d, 1H), 6.64 (d, 2H), 6.82 (d, 2H), 7.13 (d, 2H), 7.37 (d, 2H); LC-MS (m/z) calcd: 885.4. found 886.5 [M+H]⁺.

### Example 14

### De Novo Synthesis of PEG-Tipranavir

PEG-tipranavir was prepared. Schematically, the approach followed for this example is shown below (wherein Xa stands for oxazolidinone and compound numbers in bold in the schematic correspond to the compound numbers provided in the text of this Example 14 alone).

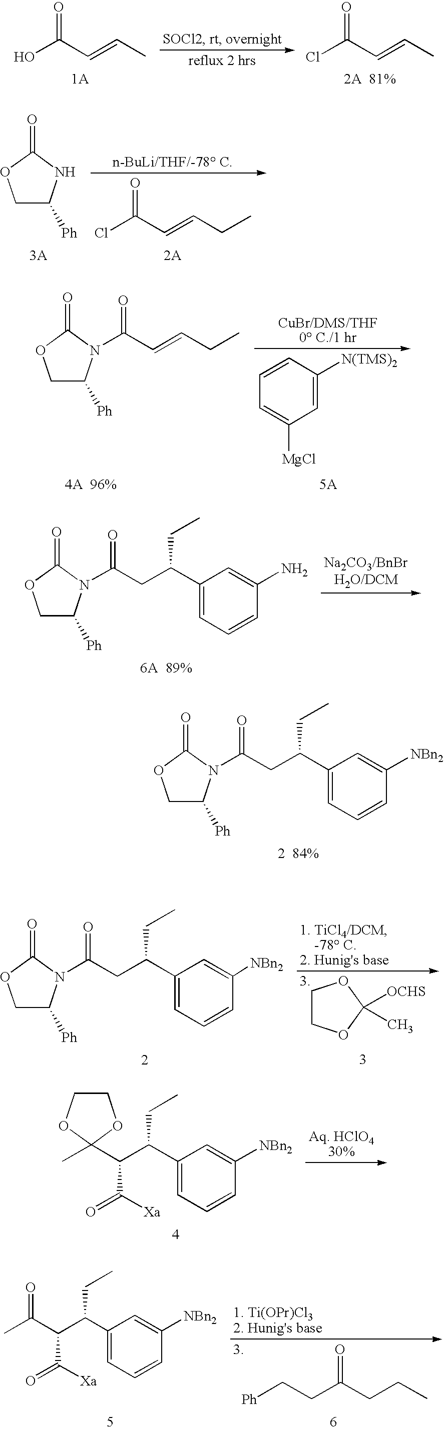

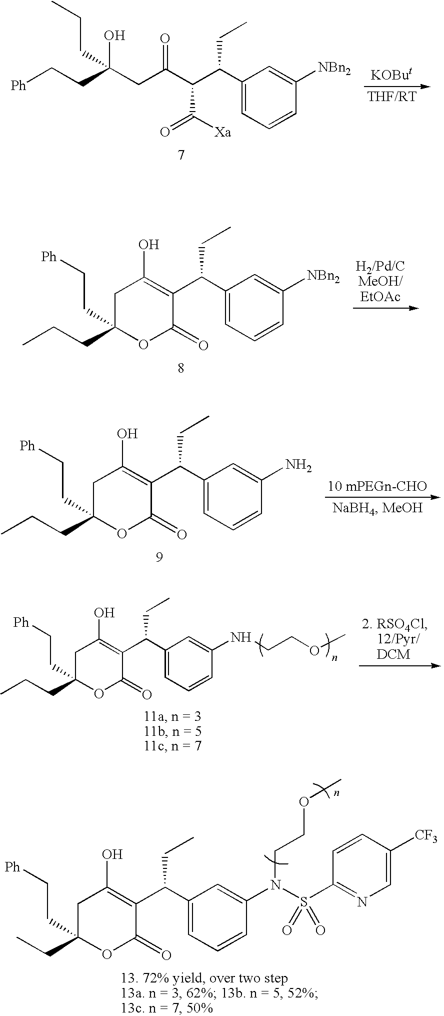

In carrying out this synthesis, the following materials were used. Calcium hydride (CaH₂), ethylene glycol, trimethyl orthoacetate, sodium hydroxide, titanium (IV) chloride, N,N-diisopropylethylamine (DIPEA), perchlorid acid 60% (HCLO₄), phenethylmagnesium chloride (1.0 M in THF), butyaldehyde, pyridinium chlorochromate (PCC), titanium (IV) isopropoxide, potassium tert-butoxide (KOBut), palladium/carbon (10 wt %), oxalyl chloride [(COCl)₂], dimethylsulfoxide (DMSO), anhydrous methanol, sodium bronohydride (NaBH₄), and pyridine were purchased from Sigma-Aldrich (St Louis, Mo.). mPEGₙ-OH (n=3, 5, 7) were purchased from TCI America. 5-Trifluoromethyl-2-pyridinesulfonyl chloride was purchased from Toronto Research Chemicals, Inc. (North York, ON, Canada). DCM was distilled from CaH₂. Tetrahydrofuran (THF) and other organic solvents were used as they purchased. 2-(E)-pentenoic acid, thionyl chloride, (R)-(−)-4-phenyl-2-oxazolidinone, n-butyl lithium (1M, Hexane), 3-bis(trimethylsilyl)amino)phenylmagnesium chloride (1.0 M, THF), copper bromide (I)-dimethyl sulfide, benzyl bromide, and ammonium chloride were purchased from Sigma-Aldrich (St Louis, Mo.). Ammonium hydroxide, sodium sulfate, ethyl acetate, and hexane were purchased from Fisher Scientific (Fair Lawn, N.J.). Magnesium sulfate, sodium bicarbonate, and sodium carbonate were purchased from EM Science (Gibbstown, N.J.). DCM was distilled from CaH₂. THF (anhydrous) and acetonitrile were also purchased from Sigma-Aldrich and used as purchased.

Acid Chloride Preparation (2A)

In a 100-mL flask equipped with a reflux condenser, 2-(E)-pentenoic acid (15.4 mL, 152 mmol) was added under N₂. After the reaction flask was set up in a water bath, thionyl chloride (10.5 mL, 144 mmol) was then added slowly and the reaction was kept in the water bath for an additional ten minutes before it was removed and allowed to warm to room temperature. The reaction was kept at room temperature overnight and then heated to 110° C. (external) in oil bath for 30 minutes and was kept at this temperature for an additional 30 minutes. The solution was cool down below 40° C. before vacuum distillation was started. Vacuum distillation provided desired product 2 (13.8 g, 81% yield) as a colorless liquid, under 45-55° C. (external)/8 mmHg. ¹H NMR (300 MHz, CDCl₃) δ 1.13 (t, 3H, J=7.5 Hz), 2.29-2.39 (m, 2H), 6.07 (dt, 1H, J=1.5, 15.3 Hz), 7.28 (dt, 1H, J=6.3, 15.3 Hz).

Oxazolidinone Amide Bond Formation (4A)

Oxazolidinone (3A) (6.90 g, 42.3 mmol) was added to a 500-mL flask protected with N₂ and was filled with anhydrous THF (265 mL). The THF solution was cooled down to −78° C. in a dry-ice bath. Then n-BuLi (1.6 M in hexane, 27.8 mL, 44.4 mmol) was added slowly (about 12 minutes). The reaction was kept at this temperature for 30 minutes before 2-(E)-pentenoic acid chloride (2A) (5.51 g, 46.5 mmol) was added slowly over seven minutes. The dry-ice bath was immediately removed after addition of the acid chloride was completed and the reaction solution was warmed to room temperature over 40 minutes. The reaction then was quenched by a saturated solution of NH₄Cl (400 mL). A small amount of pure de-ionized water was added to dissolve the precipitation of NH₄Cl. The organic THF phase was separated and the aqueous phase was extracted with EtOAc (100 mL×2). The organic phases were combined, dried over MgSO₄, and concentrated to about 25 mL. While stirring, hexane (200 mL) was added and the crude product precipitated in a few minutes. After filtration, the solution was concentrated to about 10 mL and precipitated a second time with hexane (about 180 mL). The mother liquor was concentrated and the resulting residue was purified on Biotage (EtOAc/Hex 6-50% in 20 CV). Three portions of colorless product (4A) were combined (9.95 g, 96% yield). R_{f}=0.45 (Hex:EtOAc=3:1), RP-HPLC (betasil C18, 0.5 mL/min, 60-100% ACN in 8 min) 7.40 min, LC-MS (ESI, MH⁺) 246.1. ¹H NMR (300 MHz, CDCl₃) δ 1.08 (t, 3H, J=7.5 Hz), 2.28 (p, 2H, J=6.3 Hz), 4.28 (dd, 1H, J=3.9, 9.0 Hz), 4.70 (t, 1H, J=8.7 Hz), 5.49 (dd, 1H, J=3.9, 8.7 Hz), 7.09-7.18 (m, 1H), 7.23-7.42 (m, 6H).

Asymmetric Michael Addition:

In a 500-mL flask protected with N₂, copper bromide (I)-dimethyl sulfide (7.44 g, 36.2 mmol) was added followed by anhydrous THF (75 mL). The solution was cooled down to −45° C. with dry-ice/acetonitrile before 3-[bis(trimethylsilyl)amino]-phenylmagnesium chloride (1.0 M, 36.2 mL, 36.2 mmol) was added dropwise over 30 minutes. The reaction was kept at a temperature between −40° C. to 0° C. for 20 minutes. A solution of above starting material (4A) (7.1 g, 29.0 mmol) in THF (19.3 mL) was added dropwise over 20 minutes. The reaction then was warmed to 0° C. over 10 min and then further to room temperature over 15 minutes. The reaction mixture was quenched with the addition of aqueous NH₄Cl (70 mL) at room temperature for 15 minutes. The aqueous phase was then adjusted to pH=8 by addition of NH₄OH (5 mL). The solution was then poured into an ether solution (250 mL) and the aqueous phase was separated. The ether phase was washed with NaHCO₃ (80 mL×2) until the aqueous phase was not blue to pH paper anymore. The ether phase was then dried over Na₂SO₄ and concentrated in vacuo. The resulting residue was loaded on the reverse phase column (40 M x 3, about 8 g crude each) and purified via 20-70% ACN in 20 CV. Fractions were collected and acetonitrile was evaporated. The aqueous phase then extracted with DCM (50 mL×3). The organic solution was combined, dried over Na₂SO₄, concentrated to give product (6A) (8.73 g, 89% yield). R_{f}=0.11 (Hex:EtOAc=3:1), RP-HPLC (betasil C18, 0.5 mL/min, 60-100% ACN in 8 min) 5.67 min, LC-MS (ESI, MH⁺) 339.2. ¹H NMR (500 MHz, CDCl₃) δ 0.76 (t, 3H, J=7.2 Hz), 1.50-1.68 (m, 2H), 2.90-3.00 (m, 1H), 3.06 (dd, 1H, J=7.2, 15.6 Hz), 3.48 (dd, 1H, J=7.5, 15.6 Hz), 4.17 (dd, 1H, J=4.2, 9.3 Hz), 4.64 (t, 1H, J=9.0 Hz), 5.38 (dd, 1H, J=3.9, 8.7 Hz), 6.51-6.61 (m, 3H), 6.99-7.07 (m, 3H), 7.22-7.28 (m, 3H).

Benzyl Protection of Amine:

The above product (6A) (13.5 g, 40 mmol) was dissolved in DCM (146 mL) and H₂O (106 mL) in a 500-mL flask. Solid sodium carbonate (25 g, 240 mmol) and benzyl bromide (19.0 mL, 160 mmol) were added. The solution was heated (52° C. external) to reflux overnight (20 hrs) before it was checked by TLC. The reaction was diluted with NaHCO₃ (300 mL) and DCM was separated from the solution. The aqueous phase was then extracted with DCM (60 mL×2) and organic phases were combined. The solution was dried over Na₂SO₄ and concentrated. The residue was loaded on Biotage (40 M×2, 14 g crude each) over 6-22% EtOAc/Hex in 18 CV. The product fractions were collected and evaporated to generate a colorless soft solid product (2) (17.5 g, 84%). R_{f}=0.42 (Hex:EtOAc=3:1), RP-HPLC (betasil C18, 0.5 mL/min, 60-100% ACN in 8 min, 100% 8-12 min) 9.80 min, LC-MS (ESI, MH⁺) 519.2. ¹H NMR (300 MHz, CDCl₃) δ 0.65 (t, 3H, J=7.2 Hz), 1.40-1.55 (m, 2H), 2.84-2.94 (m, 1H), 3.02 (dd, 1H, J=7.2, 15.6 Hz), 3.42 (dd, 1H, J=7.5, 15.6 Hz), 4.15 (dd, 1H, J=3.9, 8.7 Hz), 4.53-4.67 (m, 5H), 5.35 (dd, 1H, J=3.9, 8.7 Hz), 6.50-6.61 (m, 3H), 6.98-7.07 (m, 3H), 7.18-7.29 (m, 13H).

### Synthesis of Glycol Ortho Ester

### Compound (3)

A fresh CaH₂ distilled starting material (26.3 g, 219 mmol) was mixed with ethylene glycol (11 mL, 197 mmol) at room temperature. H₂SO₄ (3-4 drops, 0.25%) was added and stirring at this temperature. A water spray vacuo system with a solid NaOH dry bottle and a mercury manometer was set up to the distillation reaction system. The vacuo was adjusted below 95 mmHg (not less than 55 mmHg) and the temperature was gradually increased (10° C. per ten minutes). After a forerun (˜2 g) was collected, a colorless product (16.2 g, 70% yield) was collected under 68-71° C./58-60 mmHg. NMR (300 MHz, CDCl₃) δ 1.55 (3H, s), 3.28 (3H, s), 3.97-4.12 (4H, m).

### TiCl₄ Activated C—C Conjugation to Prepare Compound (4)

A pre-vacuo dried starting material (2) (6.45 g, 12.4 mmol) was dissolved in DCM (50 mL) under the protection of N₂. It was then allowed to cool down to −78° C. in a dry-ice/acetone bath. TiCl₄ (2.45 mL, 22.3 mmol) was dropwise added and the reaction at this temperature was kept for five minutes before DIPEA (4.11 mL, 23.6 mmol) was added. The bath was moved away immediately and the reaction was warm up to 0° C. in salt-ice bath. The enolate formation was kept at this temperature for 30 minutes before it was re-cooled down to −78° C. Glycol ortho ester (3) (3.66 mL, 31 mmol) was added slowly. After addition, the reaction was warm up to 0° C. and kept at this temperature for 2.5 hours. The reaction was quenched with half saturated NH₄Cl and water. The solution was diluted with water and extracted with DCM (50 mL×3). The combined organic phase was washed with NaHCO₃ and dried over Na₂SO₄. TLC show the reaction was clean but ˜10% starting material remaining. Biotage purification (40 M×5 times) provided a colorless product (5.47 g, 73% yield) product without contamination. R_{f}=0.51 (Hex:EtOAc=3:1), LC-MS (ESI, MH⁺) 605.3. ¹H NMR (300 MHz, CDCl₃) δ_{—}δ 0.55 (3H, t, J=7.2 Hz), 0.86 (3H, s), 1.40-1.51 (2H, m) 2.89 (1H, dt, J=3.6, 11.1 Hz), 3.03 (1H, q, J=6.9 Hz), 3.44-3.50 (1H, m), 3.54 (1H, q, J=6.9 Hz), 3.62-3.72 (1H, m), 4.26 (1H, dd, J=3.6, 9.0 Hz), 4.55-4.67 (5H, m), 4.80 (1H, d, J=10.8 Hz), 5.46 (1H, dd, J=3.3, 8.4 Hz), 6.59-6.63 (3H, m), 7.08 (1H, t, J=7.5 Hz), 7.19-7.37 (15H, m).

### Acid Hydrolysis of Acetal to Form Compound (5)

The acetal product (4) (5.47 g, 9.06 mmol) was dissolved in anhydrous THF (18 mL). Deionized water (3.6 mL) and HClO₄ (3.6 mL) were added. The reaction was started in an oil bath at the temperature of 40° C. (external) for 2.5 hours. After cooling down to room temperature, the solution was neutralized with NaHCO₃ slowly to pH=8˜9. The mixture solution was diluted with water (100 mL) and extracted with DCM (80 mL×3). The organic phase was dried over Na₂SO₄ and concentrated in vacuo. The residue was loaded on Biotage column (25M) with gradient elute (4-13% EtOAc/Hex in 16 CV). A colorless solid (5.18 g, >100% yield) was collected after high vacuo drying. R_{f}=0.43 (Hex:EtOAc=3:1), RP-HPLC (betasil C18, 0.5 mL/min, 60-100% ACN in 10 min) 6.40 min, LC-MS (ESI, MH⁺) 561.3. ¹H NMR (300 MHz, CDCl₃) δ 0.61 (3H, t, J=7.2 Hz), 1.63 (3H, s), 1.07 (1H, dt, J=3.3, 10.8 Hz), 4.22 (1H, dd, J=3.9, 8.7 Hz), 4.61 (4H, s), 4.67 (1H, t, J=9.0 Hz), 4.98 (1H, d, J=10.5 Hz), 5.42 (1H, dd, J=3.6, 8.7 Hz), 6.54-6.64 (3H, m), 7.09 (1H, t, J=8.1 Hz), 7.21-7.39 (15H, m).

### Synthesis of Compound (6)

Phenyl ethyl magnesium chloride (1M in THF, 120 mmol) was cannulated to a 500-mL flask together with THF (180 mL). The above mixture solution was then cool down to 0° C. using an ice-water bath before butyraldehyde (10.2 mL, 114 mmol) was added dropwise. TLC indicated a clean reaction after one hour at this temperature. The reaction was then quenched with NH₄Cl (150 mL) and the THF was separated. The THF solution was washed with saturated brine before it was dried over Na₂SO₄ and concentrated in vacuo. Over 20 grams of the secondary alcohol product (>100% yield) was obtained without further purification.

The secondary alcohol product (4.56 g, 25.6 mmol) was mixed with DCM (128 mL) at room temperature. PCC (6.62 g, 30.7 mmol) was added. The reaction was kept at room temperature for two hours. Due to the TLC indicated an about 15% of remaining starting material, another part of PCC (1.11 g, 5.1 mmol) was added and the reaction was finished in two hours. The solution mixture was filtrated though a layer of celite and silica gel. The filtrated solution was then evaporated and the residue was purified on Biotage column (40S). A colorless compound (6) (2.79 g, yield 62%) was collected. NMR proton spectrum indicated a product with impurity <1%. ¹H NMR (300 MHz, CDCl₃) δ 0.89 (3H, t, J=7.2 Hz), 1.56-1.63 (2H, m), 2.37 (2H, t, J=7.2 Hz), 2.72 (2H, t, J=7.2 Hz), 2.90 (2H, t, J=7.5 Hz), 7.17-7.21 (3H, m), 7.26-7.28 (2H, m).

### Ti-Activated C—C Conjugation

### Aldol Reaction to Form Compound (7)

In a N₂ protected 100-mL flask, freshly distilled DCM (22 mL) was added. Ti(OPr)₄ (373 μL, 1.27 mmol) and TiCl₄ (377 μL, 3.44 mmol) were added in that order. The reaction solution was cooled down to −78° C. in an acetone-dry ice bath and compound (5) (1.93 g, 3.44 mmol) in DCM (6 mL) solution was added slowly. The solution was reddish and was kept at this temperature for five minutes before DIPEA (899 μL, 5.16 mmol) was added. The acetone-dry ice bath was taken away and warmed to 0° C. before an ice-water bath was used. The enolate formation was kept at 0° C. for one hour before it was re-cooled down to −78° C. in an acetone-dry ice bath. Compound (6) (1.21 mL, 6.88 mmol) was added slowly. The solution was then warm up to 0° C. and kept at this temperature via ice-water bath for one hour. The reaction was quenched by saturated NH₄Cl solution (30 mL) and a diluted mixture was extracted by DCM (40 mL×3). The combined organic phase was then dried over Na₂SO₄ and concentrated in vacuo. The residue was loaded on the Biotage column (40S) with a gradient (8-18% EtOAc/Hex in 16 CV). A yellowish product (1.90 g, yield 75%) was collected. R_{f}=0.42 (Hex:EtOAc=3:1), RP-HPLC (betasil C18, 0.5 mL/minute, 60-100% ACN in ten minutes) 9.13 minutes, LC-MS (ESI, MH⁺) 737.5.

### Basic Hydrolysis and Lactonization to Synthesize Compound (8)

The aldol product (7) mixture (1.68 g, 2.28 mmol) was dissolved in the THF (50 mL) under a N₂ atmosphere. After the sample was dissolved, the solution was allowed to cool in ice-water bath for five minutes before KOBu^{t} (1 M, 2.74 mL) was added. The reaction was kept at this temperature for 20 minutes. It was quenched with NH₄Cl (50 mL) and the organic phase was diluted with EtOAc (150 mL). The aqueous phase was then separated (ensuring a pH<7) and the ether phase was washed with saturated brine (50 mL). It was then dried over Na₂SO₄ and concentrated under vacuo. The dried residue was then loaded on Biotage column (25 M) and purified (6-22% EtOAc/Hex in 16 CV) four times. The yellowish benzyl amine compound (8) (712.1 mg, yield 54.5%) was solidified after high vacuo drying. R_{f}=0.41 (Hex:EtOAc=3:1), RP-HPLC (betasil C18, 0.5 mL/minute, 60-100% ACN in ten minutes) 5.23 minutes, LC-MS (ESI, MH⁺) 574.4.

### Pd/C hydrogenation to synthesize (R)-3-(R)-1-(3-aminophenyl)propyl)-5,6-dihydro-4-hydroxy-6-phenethyl-6-propylpyran-2-one (9)

The benzyl amine compound (8) (265.8 mg, 0.464 mmol) was dissolved in EtOAc (6.5 mL) and MeOH (6.5 mL) mixture solution. The solution vial was bubbling N₂ for exchange at lease 15 minutes before catalyst addition. Stirring was stopped and the Pd/C catalyst (43 mg, 8 wt %×2) was added slowly (or in small portions). The system was evacuated and recharged with hydrogen gas (<50 psi) three times (stop stirring during vacuo). The hydrogenolysis was then kept at room temperature under 50 psi for overnight (16 hrs) to complete. After release the pressure, the reaction mixture was first checked with HPLC to see the completeness before a filtration was performed. The catalyst residue and filter paper were carefully washed with methanol. The solution was then evaporated and vacuo drying to give oil-like compound (9) (182 mg, 100% yield). No further purification is necessary. RP-HPLC (betasil C18, 0.5 mL/minute, 10-100% ACN in 8 minutes) 4.58 minutes, LC-MS (ESI, MH⁺) 394.2.

### Preparation of Compound (10) Via Swern Oxidation of mPEGn-OH

In a 250-mL flask with N₂ protection, DCM (105 mL) and oxalyl chloride (2M, 7.5 mL, 15 mmol) was added. The solution was cool down to −78° C. in dry-ice acetone bath for five minutes before DMSO (1.42 mL, 20.0 mmol) was added. It was stirred vigorously at this temperature for 20 minutes before a mPEG₇-OH (3.40 g, 10.0 mmol) and DCM (10 mL) mixture was added. The reaction was kept at this temperature for another 20 minutes before TEA (5.5 mL, 39.6 mmol) were added. The reaction was kept in dry-ice bath for three minutes and the bath was removed to gradually warm up to ambient temperature for 25 minutes. It was quenched with saturated NaHCO₃ (70 mL) and DCM solution was diluted (120 mL). The organic phase was separated and aqueous phase was extracted with DCM (20 mL×2). It was dried over Na₂SO₄ and then concentrated and a slight yellow liquid with some solid inside (2.78 g, 82% yield) was saved in N₂. NMR indicated a 64% conversion mixture. Biotage FCC (3-10% MeOH in DCM in 16 CV) provided pure product for reductive amination. R_{f}=0.32 (DCM:MeOH=10:1), ¹H NMR (300 MHz, CDCl₃) δ 3.39 (s, 3H), 3.54-3.57 (m, 2H), 3.66 (s, 20H), 3.72-3.75 (m, 2H), 4.17 (s, 2H), 9.74 (s, 1H).

mPEG₅-CHO was synthesized in a similar approach. Crude product showed 86% aldehyde with 99% yield. Biotage FCC (3-10% MeOH in DCM in 16 CV) provided 75% aldehyde product with 56% yield and 15% aldehyde mixture with 25% yield. R_{f}=0.34 (DCM:MeOH=10:1), ¹H NMR (300 MHz, CDCl₃) δ 3.38 (s, 3H), 3.38-3.57 (m, 2H), 3.67 (s, 11H), 3.70-3.75 (m, 3H), 4.17 (s, 2H), 9.74 (s, 1H).

### Reductive Amination to Synthesize Compound (11)

Compound (9) (69.6 mg, 0.177 mmol) was dissolved in methanol (3.4 mL). While stirring, mPEG₅-CHO (235 mg, 75% purity, 0.708 mmol) was added dropwise. The reaction was run for 18 minutes and thereafter moved to a water bath at ambient temperature. NaBH₄ (54 mg, 1.42 mmol) was added in several portions. HPLC was used to check the reaction after three minutes and evidenced the reaction achieved 77% conversion. The reaction was quenched with NaHCO₃ (10 mL) and diluted with water and EtOAc. The organic phase was then separated and dried over Na₂SO₄. HPLC show the reaction has 81% conversion with 13% of starting material remaining. The solution was diluted with NaHCO₃ aqueous solution and extracted with DCM (30 mL×3). The combined organic solution was evaporated to provide crude sample (178 mg). It was dissolved in ACN (6 mL) and water (2 mL) and purified on AKTA (40-57% in 5 CV×2, 12.10 minutes). The acetonitrile solution of collected product was evaporated and saturated with NaCl. It was extracted with DCM (30 mL×3) and combined solution was dried over NaSO₄, filtrated, concentrated under vacuo. A slightly yellowish product (75.9 mg, 69% yield) was obtained with purity over 99%. RP-HPLC (betasil C18, 0.5 mL/minute, 30-100% ACN in ten minutes) 5.53 minutes, LC-MS (ESI, MH⁺) 628.2.

This synthetic procedure was followed except mPEG₃-CHO was substituted for mPEG₅-CHO. With excess aldehyde (1.6 eq), the product mixture showed 72% of conversion after work up. AKTA purification (40-50% ACN in 3 CV, 13.2 minutes) provided 42% yield product with purity over >99%. RP-HPLC (betasil C18, 0.5 mL/minutes, 30-100% ACN in ten minutes) 5.65 minutes, LC-MS (ESI, MH⁺) 540.3.

This synthetic procedure was followed except mPEG₇-CHO was substituted for mPEG₅-CHO. With excess aldehyde (4.5 eq), the product mixture showed 78% of conversion after work up. AKTA purification (40-57% in 5 CV) provided 73% yield of pure product (>99%). RP-HPLC (betasil C18, 0.5 mL/minute, 60-100% ACN in eight minutes) 5.06 minutes, LC-MS (ESI, MH⁺) 716.4.

### Synthesis of Compound (13a)

The above AKTA purified product (11a) (96.8 mg, 0.180 mmol) was dissolved in DCM (1.6 mL). After dissolving, the solution was cool down in an ice-water bath and trifluoropyridine sulphonyl chloride (48.6 mg, 0.198 mmol) was added. Pyridine (44 μL, 0.54 mmol) was then added and the reaction was warm up during the overnight reaction. HPLC showed the retention time of starting material was completed and the reaction was quenched with NH₄Cl (10 mL). It was diluted with DCM and the separated organic phase was washed with brine. The organic phase was then dried over Na₂SO₄ and concentrated. The crude product (159.4 mg) was purified on Biotage (10-50% EtOAc in Hex with 16 CV) provided a slightly yellowish product (13a) (73.1 mg) and a less pure product (35.7 mg) with the total yield about 62%. R_{f}=0.22 (Hex:EtOAc=1:1), RP-HPLC (betasil C18, 0.5 mL/minute, 60-100% ACN in 8 minutes) 4.20 minutes, LC-MS (ESI, MH⁺) 749.3.

Following a procedure similar to the synthesis of compound (13a), compound (11b) (154.9 mg) produced the desired product (13b) (36.0 mg, 93% pure) and a mixture of product (71.2 mg) with a yield of −52%. Purification over Biotage silical gel column (1-7% MeOH in DCM in 16 CV). R_{f}=0.54 (EtOAc), RP-HPLC (betasil C18, 0.5 mL/minute, 60-100% ACN in 8 minutes) 4.36 minutes, LC-MS (ESI, MH⁺) 837.4.

Following a procedure similar to the synthesis of compound (13a), compound (11c) (167.7 mg) produced the desired product (13c) (39.9 mg, 95% pure) and a mixture of product (82.3 mg) with a yield of ˜50%. Purification over Biotage silical gel column (2-7% MeOH in DCM in 16 CV). R_{f}=0.25 (EtOAc), RP-HPLC (betasil C18, 0.5 mL/minute, 60-100% ACN in 8 minutes) 3.78 minutes, LC-MS (ESI, MH⁺) 925.5.

What is claimed is:

1. A method of synthesizing a conjugate of a pharmaceutically active compound, the method comprising:
attaching at least one water-soluble oligomer, directly or through a linker group, at one or more synthetically available positions within an intermediate compound; and
completing a synthetic path to yield the conjugate of the pharmaceutically active compound.

2. A method of synthesizing a conjugate of a pharmaceutically active compound, the method comprising:
selecting a pharmaceutically active compound having a synthetic path;
modifying the synthetic path by attaching at least one oligoethylene glycol residue, directly or through a linker group, at one or more synthetically available positions within one or more intermediate compounds of the synthetic path; and
completing the synthetic path to yield the conjugate of the pharmaceutically active compound.

3. The method of claim 1, wherein the synthetic path is a convergent path having two intermediate compounds that are reacted to yield the pharmaceutically active compound or a protected form of the pharmaceutically active compound, wherein the synthetically available position is within at least one of the two intermediate compounds.

4. The method of claim 3, wherein said attaching occurs at a synthetically available position within the two intermediate compounds.

5. The method of claim 1, wherein the synthetic path is a linear path.

6. The method of claim 1, wherein the water-soluble oligomer is an oligoethylene glycol residue.

7. The method of claim 6, wherein the one or more synthetically available positions is a carboxylic acid group, and the oligoethylene glycol residue is attached by esterification.

8. The method of claim 6, wherein the one or more synthetically available positions is an ester group, and the oligoethylene glycol residue is attached by transesterification.

9. The method of claim 6, wherein the one or more synthetically available positions is a hydroxy group, and the oligoethylene glycol residue is attached by etherification.

10. The method of claim 6, wherein the one or more synthetically available positions is an amino group, and the oligoethylene glycol residue is attached by imine formation.

11. The method of claim 6, wherein each oligoethylene glycol residues is introduced by contacting one or more intermediate compounds at one or more synthetically available positions with one or more compounds each independently having the formula: 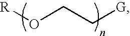
wherein:
n is an integer having a value from 2 to 50;
R is selected from the group consisting of —OH, C₁-C₁₀ alkyl and hydroxy-protecting groups; and
G is a selected from the group consisting of nucleophilic leaving groups, —OH, —SH, —NH₂, —NH(C₁-C₆ alkyl), —C(O)OH, —C(O)OC₁-C₆alkyl, and activated carboxylic acid groups.

12. The method of claim 11, wherein each synthetically available position in the one or more intermediate compounds independently comprises a hydrogen having a pKa of less than about 25.

13. The method of claim 6, wherein each oligoethylene glycol residue is introduced by contacting one or more intermediate compounds at one or more synthetically available positions with one or more compounds each independently having the formula, 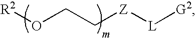
wherein:
m is an integer having a value from 2 to 50;
Z is —O— or —N(H)—;
R² is selected from the group consisting of —OH, C₁-C₁₀ alkyl and hydroxy-protecting groups;
L is selected from the group consisting of —C(O)—, —C₁-C₆ alkyl-, —C(O)C₁-C₆ alkyl-, —C(O)OC₁-C₆ alkyl-, or —C(O)N(H)C₁-C₆ alkyl-; and
G² is selected from the group consisting of halogen, —OH, —SH, —NH₂, —NH(C₁-C₆ alkyl), —C(O)OH, —C(O)OC₁-C₆alkyl, and an activated carboxylic acid groups.

14. The method of claim 1, wherein each oligoethylene glycol residue is independently attached to the one or more intermediate compounds through a bond selected from the group consisting of a thioether bond, an ether bond, an ester bond, an amide bond, a carbonate bond, a carbamate bond, a urea bond, and an amino bond.

15. The method of claim 1, wherein said attaching at one or more synthetically available positions within an intermediate compound corresponds to one or more not synthetically available positions in the pharmaceutically active compound.

16. The method of claim 15, wherein the method is carried out without protecting one or more functional groups in the pharmaceutically active compound.

17. The method of claim 1, wherein the method is carried out without protecting one or more functional groups in the pharmaceutically active compound.

18. The method of claim 1, wherein the pharmaceutically active compound is nifedipine and one intermediate compound is ethyl acetoacetate.

19. The method of claim 1, wherein the pharmaceutically active compound is nifedipine and one intermediate compound is 2-nitrobenzylaldehyde.

20. The method of claim 1, wherein the pharmaceutically active compound is verapamil and one intermediate compound is homovanillyl alcohol.

21. The method of claim 1, wherein the pharmaceutically active compound is verapamil and one intermediate compound is 4-hydroxy-3-methoxyphenylacetonitrile.

22. The method of claim 1, wherein the pharmaceutically active compound is dantrolene and one intermediate compound is 2-amino-5-nitrophenol.

23. The method of claim 1, wherein the pharmaceutically active compound is oxybutynin and one intermediate compound contains a 4-(tetrahydro-pyran-2-yloxy)-but-2-ynyl group and a leaving group.

24. The method of claim 23, wherein the leaving group is selected from the group consisting of sulphonate esters and halogens.

25. The method of claim 1, wherein the pharmaceutically active compound is atazanavir and one intermediate compound is 3-amino-4-phenyl-1-[N-(4-pyridin-2-yl-benzyl)-hydrazino]-butan-2-ol.

26. The method of claim 1, wherein the pharmaceutically active compound is darunavir and one intermediate compound has the following structure:

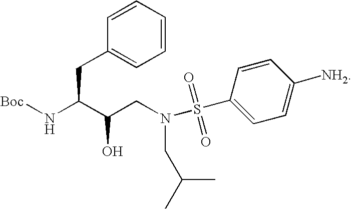

27. The method of claim 1, wherein the pharmaceutically active compound is darunavir and one intermediate compound has the following structure:

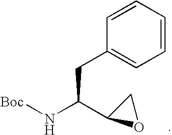

28. The method of claim 1, wherein the pharmaceutically active compound is tipranavir and one intermediate compound is (R)-3-((R)-1-(3-aminophenyl)propyl)-5,6-dihydro-4-hydroxy-6-phenethyl-6-propylpyran-2-one.

29. The method of claim 1, wherein the pharmaceutically active compound is selected from the group consisting of nifedipine, verapamil, dantrolene, oxybutynin, BW373U86, atazanavir, darunavir and tipranavir.

30. A conjugate prepared in accordance with the method of claim 1.

31. A conjugate prepared in accordance with the method of claim 2.

32. A pharmaceutical preparation comprising the conjugate of claim 30.

33. A pharmaceutical preparation comprising the conjugate of claim 31.
